(19) United States
(12) Patent Application Publication
(10) Pub. No.: US 2026/0217750 A1
(43) Pub. Date: Jul. 30, 2026

(54) SPIRO-CONTAINING PLATINUM COMPOUND AND ORGANIC LIGHT-EMITTING DEVICE

(71) Applicant: GUANGDONG AGLAIA OPTOELECTRONIC MATERIALS CO., LTD, Guangdong (CN)

(72) Inventors: Xinwei WU, Guangdong (CN); Haijian TAN, Guangdong (CN); Zhijia YANG, Guangdong (CN); Lei DAI, Guangdong (CN); Lifei CAI, Guangdong (CN)

(73) Assignee: GUANGDONG AGLAIA OPTOELECTRONIC MATERIALS CO., LTD, Guangdong (CN)

(21) Appl. No.: 19/140,209

(22) PCT Filed: Oct. 18, 2023
(86) PCT No.: PCT/CN2023/125091

§ 371 Date: Jun. 17, 2025

(30) Foreign Application Priority Data
Dec. 21, 2022 (CN) 202211646728.2

(51) Int. Cl. C07F 15/00; C07D 213/30; C09K 11/06; H10K 50/12; H10K 85/30

(52) U.S. Cl. CPC C07F 15/0086; C07D 213/30; C09K 11/06; H10K 50/12; H10K 85/346; C09K 2211/185

(57) ABSTRACT

The present invention relates to a spiro-containing platinum compound and an organic light-emitting device. The structure of the spiro-containing platinum compound is as shown in formula (I). Provided is a spiro-containing platinum compound having ONCN tetradentate ligand. The material can enhance the heavy-atom effect in phosphorescent materials, thereby improving the utilization rate of energies of both singlet and triplet excitons in the luminescence process. The spiro structure in the platinum compound provides a sufficient 3D spatial configuration, which reduces aggregation-induced quenching effects and further improves the luminous efficiency, offering significant potential for advancing the industrial application of such materials. The platinum compound, when applied in organic light-emitting diodes, exhibits high efficiency, good thermal stability, photophysical properties and device lifetime.

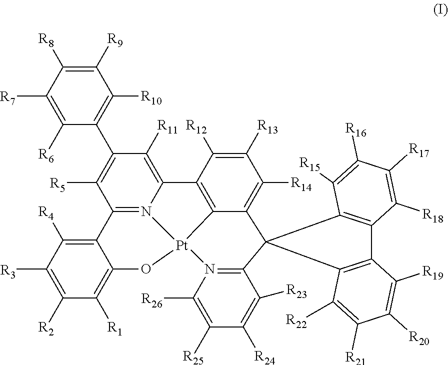

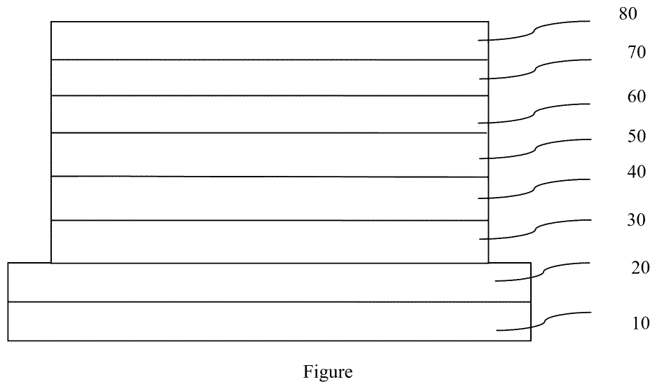

### CROSS-REFERENCE TO RELATED APPLICATIONS

The present application is a national phase entry under 35 USC § 371 of International Application No. PCT/CN2023/125091, filed Oct. 18, 2023, which claims the benefit of and priority to Chinese Patent Application No. 202211646728.2, filed Dec. 21, 2022, the entire disclosures of each of which are incorporated herein by reference.

### TECHNICAL FIELD

The present disclosure relates to the field of organic light-emitting diode (OLED) phosphorescent materials, and in particular, to a spiro-containing platinum compound and an organic light-emitting device.

### BACKGROUND

Organic light-emitting diodes (OLEDs) have the advantages of thin thickness, light weight, self-luminescence, wide viewing angles, fast response time, low energy consumption, excellent low-temperature and shock-resistance performances, and potential flexible design. As all solid-state devices devoid of vacuum chambers and liquid components, OLEDs possess shock-resistance performance and user-friendliness. Coupled with high resolution, wide viewing angles and a wide operating temperature range, OLEDs can be applied as planar backlight sources and lighting sources in the display industry. Since the reports on the electrophosphorescence phenomenon by Professor Chi-Ming Che's team from the University of Hong Kong and Forrest et al. in the United States, organic electroluminescence technology has achieved significant advances. Researchers worldwide have been committed to promoting phosphorescent materials to industrial marketization, with their research directions mainly focused on improving the performance of iridium compound phosphorescent materials. Studies have shown that organometallic compounds can achieve fast intersystem crossing (ISC) and long-lived phosphorescent decay due to their strong spin-orbit coupling (SOC), and phosphorescent materials can fully utilize the energy of both singlet and triplet excitons during the luminescence process, thereby improving the luminescence efficiency of the compounds and theoretically achieving an internal quantum efficiency of up to 100% in OLEDs. Therefore, over the past decade, organotransition metal compounds have become highly attractive for applications in electroluminescent devices such as organic light-emitting diodes (OLEDs). With this new technology, efficient light-emitting systems are being developed. In the future, it will be possible to manufacture large, bright flat-panel displays at a moderate price. An important requirement for these applications remains the realization of low power consumption and high operational efficiency in the light-emitting devices.

### SUMMARY

In view of the above problems existing in the prior art, the present disclosure provides a class of spiro-containing platinum compounds having ONCN tetradentate ligands, which can enhance the heavy-atom effect in phosphorescent materials, thereby improving the utilization rate of energies of both singlet and triplet excitons in the luminescence process. The spiro structure in the platinum compounds of the present disclosure provides a sufficient three-dimensional (3D) spatial configuration, which reduces aggregation-induced quenching effects and further improves the luminous efficiency, offering significant potential for advancing the industrial application of such materials.

The platinum compound, when applied in organic light-emitting diodes, exhibits high efficiency, good thermal stability, photophysical properties and device lifetime.

The spiro-containing platinum compound has a structure of formula (I):

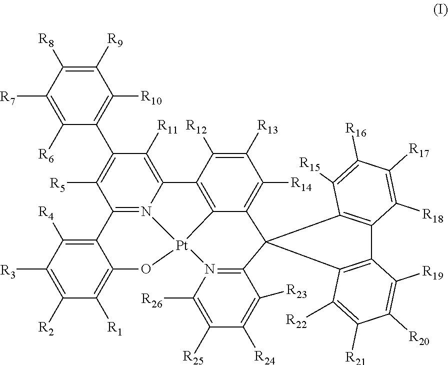

where R₁-R₂₆ are each independently selected from a group consisting of hydrogen, deuterium, halogen, a substituted or unsubstituted alkyl having 1 to 20 carbon atoms, a substituted or unsubstituted cycloalkyl having 3 to 20 ring carbon atoms, a substituted or unsubstituted heteroalkyl having 1 to 20 carbon atoms, a substituted or unsubstituted aralkyl having 7 to 30 carbon atoms, a substituted or unsubstituted alkoxy having 1 to 20 carbon atoms, a substituted or unsubstituted aryloxy having 6 to 30 carbon atoms, a substituted or unsubstituted alkenyl having 2 to 20 carbon atoms, a substituted or unsubstituted aryl having 6 to 40 carbon atoms, a substituted or unsubstituted heteroaryl having 3 to 40 carbon atoms, a substituted or unsubstituted alkylsilyl having 3 to 20 carbon atoms, a substituted or unsubstituted arylsilyl having 6 to 20 carbon atoms, a substituted or unsubstituted amino group having 0 to 20 carbon atoms, a substituted or unsubstituted acyl having 1 to 20 carbon atoms, carboxy, a substituted or unsubstituted ester group having 1 to 20 carbon atoms, cyano, isocyano, a substituted or unsubstituted sulfanyl having 0 to 20 carbon atoms, a substituted or unsubstituted sulfinyl having 0 to 20 carbon atoms, a substituted or unsubstituted sulfonyl having 0 to 20 carbon atoms, or a substituted or unsubstituted phosphino having 0 to 20 carbon atoms;
where groups with adjacent serial numbers among R₆-R₁₀ are capable of being bonded to form a ring, groups with adjacent serial numbers among R₁₅-R₁₈ are capable of being bonded to form a ring, and groups with adjacent serial numbers among R₁₉-R₂₂ are capable of being bonded to form a ring;
where the term “substituted” refers to being substituted with deuterium, halogen, amino, nitro, cyano, an aryl having 6 to 20 carbon atoms, a heteroaryl having 5 to 10 carbon atoms, or C1-C4 alkyl; and
where the heteroaryl and the heteroalkyl each contain at least one heteroatom selected from S, N or O.

At least one of R₂₃-R₂₆ is selected from a group consisting of a substituted or unsubstituted alkyl having 1 to 20 carbon atoms, a substituted or unsubstituted cycloalkyl having 3 to 20 ring carbon atoms, a substituted or unsubstituted heteroalkyl having 1 to 20 carbon atoms, a substituted or unsubstituted aralkyl having 7 to 30 carbon atoms, a substituted or unsubstituted alkoxy having 1 to 20 carbon atoms, a substituted or unsubstituted aryloxy having 6 to 30 carbon atoms, a substituted or unsubstituted alkenyl having 2 to 20 carbon atoms, a substituted or unsubstituted aryl having 6 to 40 carbon atoms, a substituted or unsubstituted heteroaryl having 3 to 40 carbon atoms, a substituted or unsubstituted alkylsilyl having 3 to 20 carbon atoms, or a substituted or unsubstituted arylsilyl having 6 to 20 carbon atoms.

In some embodiments, R₁-R₂₂ are each independently selected from a group consisting of hydrogen, deuterium, halogen, amino, carboxyl, a substituted or unsubstituted sulfanyl having 1 to 20 carbon atoms, cyano, a substituted or unsubstituted sulfonyl having 0 to 20 carbon atoms, a substituted or unsubstituted phosphino having 0 to 20 carbon atoms, a substituted or unsubstituted alkyl having 1 to 20 carbon atoms, a substituted or unsubstituted cycloalkyl having 3 to 20 ring carbon atoms, a substituted or unsubstituted aryl having 6 to 10 carbon atoms, a substituted or unsubstituted heteroaryl having 3 to 10 carbon atoms, a substituted or unsubstituted alkylsilyl having 3 to 10 carbon atoms, or a substituted or unsubstituted arylsilyl having 6 to 10 carbon atoms; and
where one of R₂₃-R₂₆ is selected from a group consisting of a substituted or unsubstituted alkyl having 1 to 20 carbon atoms, a substituted or unsubstituted cycloalkyl having 3 to 20 ring carbon atoms, a substituted or unsubstituted heteroalkyl having 1 to 20 carbon atoms, a substituted or unsubstituted aralkyl having 7 to 30 carbon atoms, a substituted or unsubstituted alkoxy having 1 to 20 carbon atoms, a substituted or unsubstituted aryloxy having 6 to 30 carbon atoms, a substituted or unsubstituted alkenyl having 2 to 20 carbon atoms, a substituted or unsubstituted aryl having 6 to 30 carbon atoms, a substituted or unsubstituted heteroaryl having 3 to 30 carbon atoms, a substituted or unsubstituted alkylsilyl having 3 to 20 carbon atoms, or a substituted or unsubstituted arylsilyl having 6 to 20 carbon atoms; and the remaining three of R₂₃-R₂₆ are hydrogen.

In some embodiments, the spiro-containing platinum compound has a structure of formula (II):

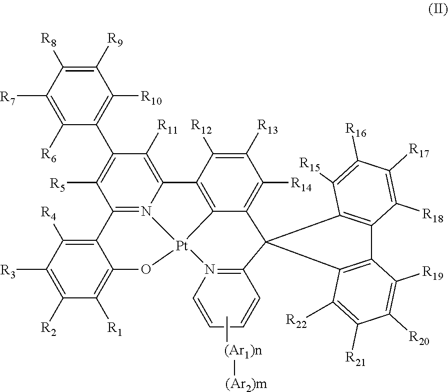

where Ar₁ and Ar₂ are each independently selected from a substituted or unsubstituted aryl having 6 to 40 carbon atoms, or a substituted or unsubstituted heteroaryl having 3 to 40 carbon atoms; n and m are integers from 0 to 2, and m+n≥1;
where R₁-R₂₂ are as defined above; and
where the term “substituted” refers to being substituted with deuterium, halogen, amino, nitro, cyano, an aryl having 6 to 20 carbon atoms, a heteroaryl having 5 to 10 carbon atoms, or C1-C4 alkyl.

Ar₁ is independently selected from a substituted or unsubstituted conjugated fused-ring aryl having 6 to 20 carbon atoms, a substituted or unsubstituted conjugated fused-ring heteroaryl having 6 to 20 carbon atoms, or a substituted or unsubstituted non-fused-ring aryl having 6 to 20 carbon atoms; and Ar₂ is independently selected from a substituted or unsubstituted aryl having 6 to 20 carbon atoms, or a substituted or unsubstituted heteroaryl having 3 to 20 carbon atoms.

In some embodiments, R₁-R₂₂ are each selected from a group consisting of hydrogen, deuterium, halogen, amino, cyano, a substituted or unsubstituted alkyl having 1 to 10 carbon atoms, a substituted or unsubstituted cycloalkyl having 3 to 10 ring carbon atoms, a substituted or unsubstituted aryl having 6 to 10 carbon atoms, a substituted or unsubstituted heteroaryl having 3 to 10 carbon atoms, a substituted or unsubstituted alkylsilyl having 3 to 10 carbon atoms, or a substituted or unsubstituted arylsilyl having 6 to 10 carbon atoms. In some embodiments, among R₁-R₂₂, R₇, R₉, R₁₃ and R₁₅-R₂₂ are each independently selected from a group consisting of hydrogen, deuterium, halogen, amino, cyano, or an alkyl group having 1 to 6 carbon atoms; and the rest of R₁₅-R₂₂ are hydrogen.

In some embodiments, Ar₁ is selected from a group consisting of a substituted or unsubstituted phenyl, a substituted or unsubstituted biphenyl, a substituted or unsubstituted naphthyl, a substituted or unsubstituted anthracyl, or a substituted or unsubstituted carbazolyl; Ar₂ is selected from a group consisting of a substituted or unsubstituted phenyl, a substituted or unsubstituted carbazolyl, a substituted or unsubstituted fluorenyl, a substituted or unsubstituted indolyl, a substituted or unsubstituted dibenzofuranyl, a substituted or unsubstituted dibenzothiophenyl, a substituted or unsubstituted acridinyl, a substituted or unsubstituted aza-anthracyl or oxo-anthracyl, or a substituted or unsubstituted phenanthrenyl; n and m are integers from 0 to 1.

In some embodiments, among R₁-R₂₂, R₇, R₉, R₁₃, R₁₆, and R₂₁ are each independently selected from hydrogen, deuterium, or an alkyl group having 1 to 6 carbon atoms; and the rest of R₁-R₂₂ are hydrogen;
where Ar₁ is selected from a substituted or unsubstituted phenyl or a substituted or unsubstituted carbazolyl; Ar₂ is selected from a substituted or unsubstituted phenyl or a substituted or unsubstituted carbazolyl; and
where the term “substituted” refers to being substituted with deuterium, halogen, amino, nitro, cyano, an aryl having 6 to 20 carbon atoms, a heteroaryl having 5 to 10 carbon atoms, or C1-C4 alkyl.

Examples of platinum compounds according to the present disclosure are listed below, but are not limited to the listed structures:

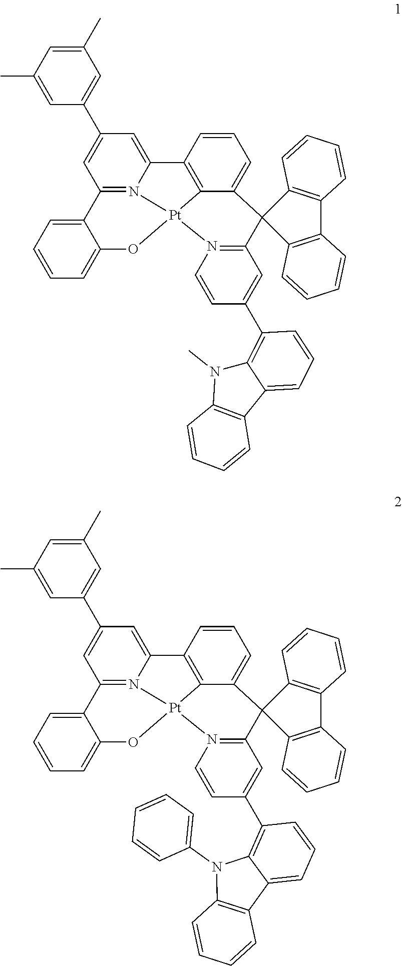

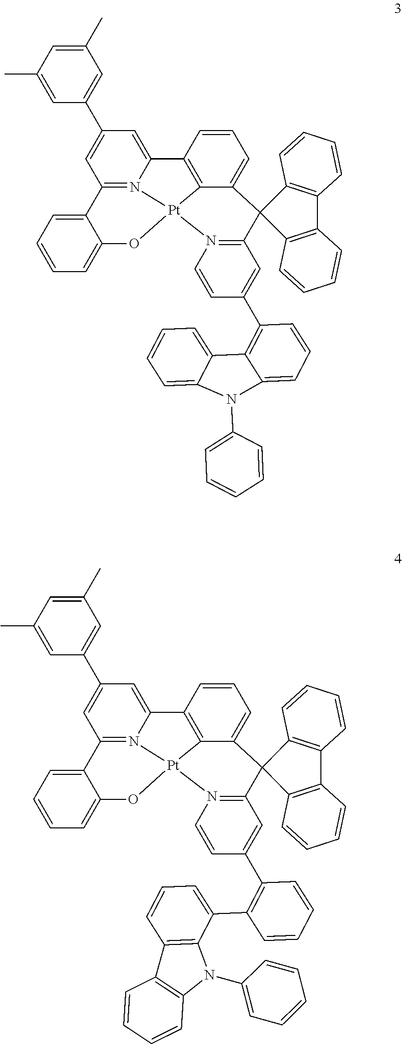

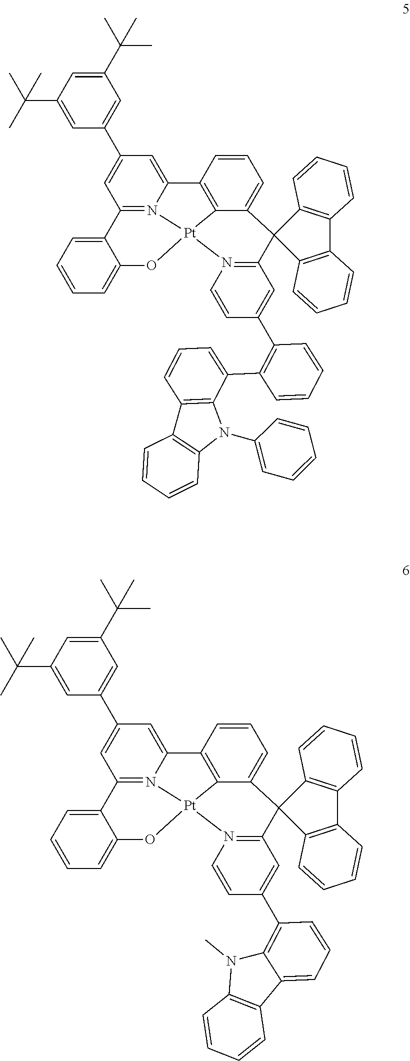

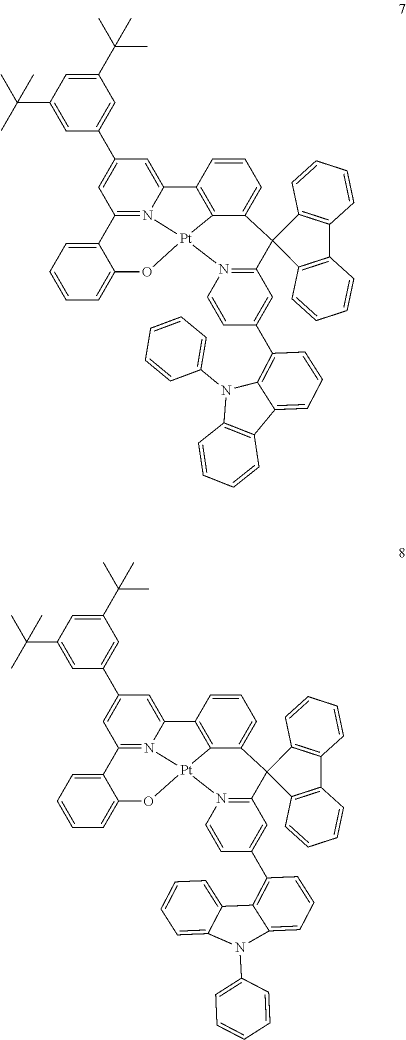

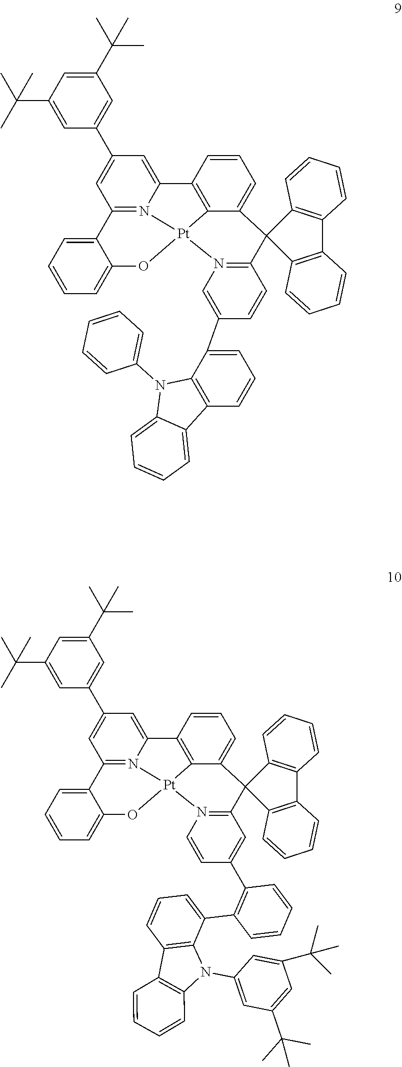

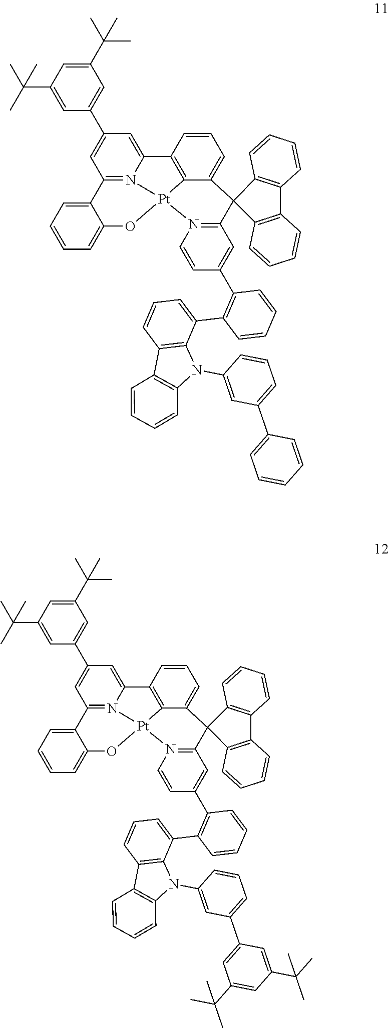

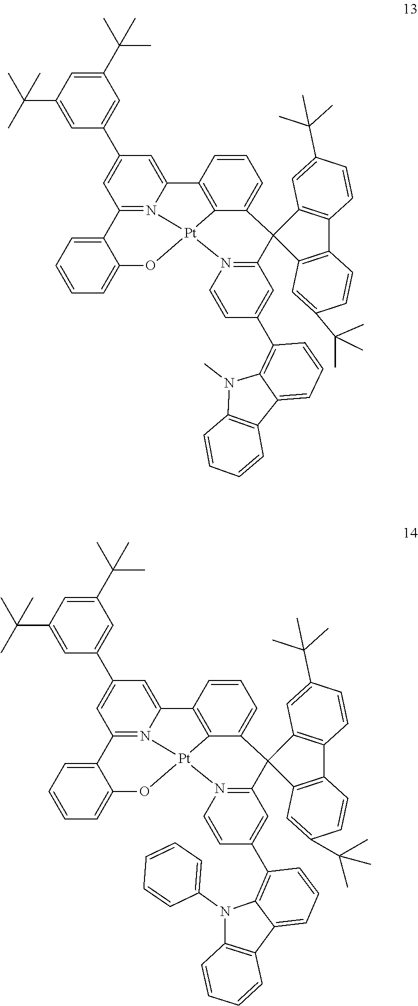

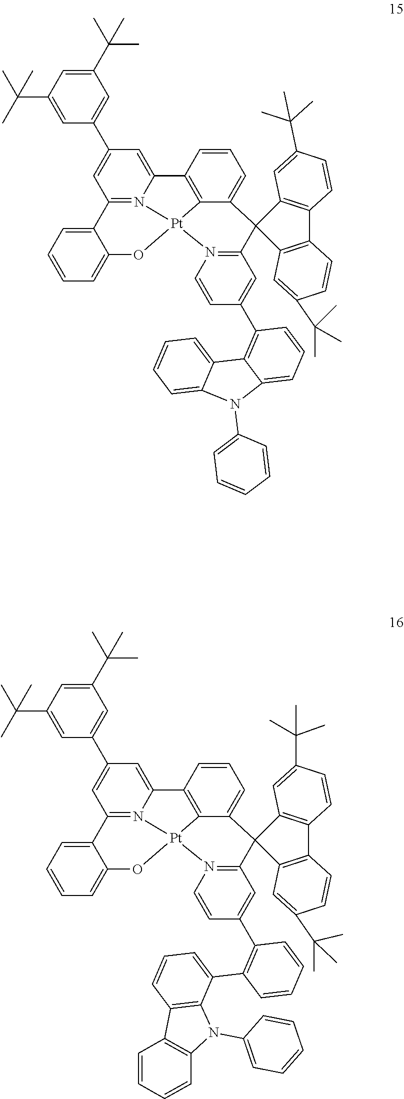

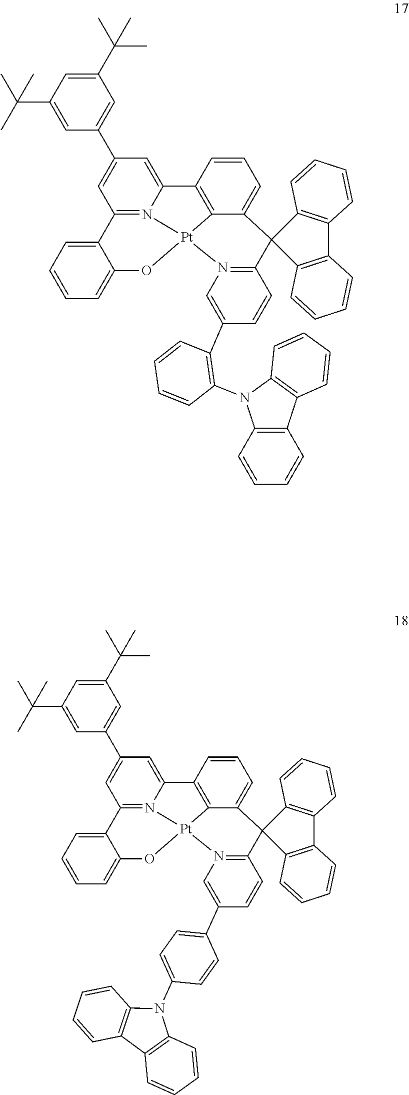

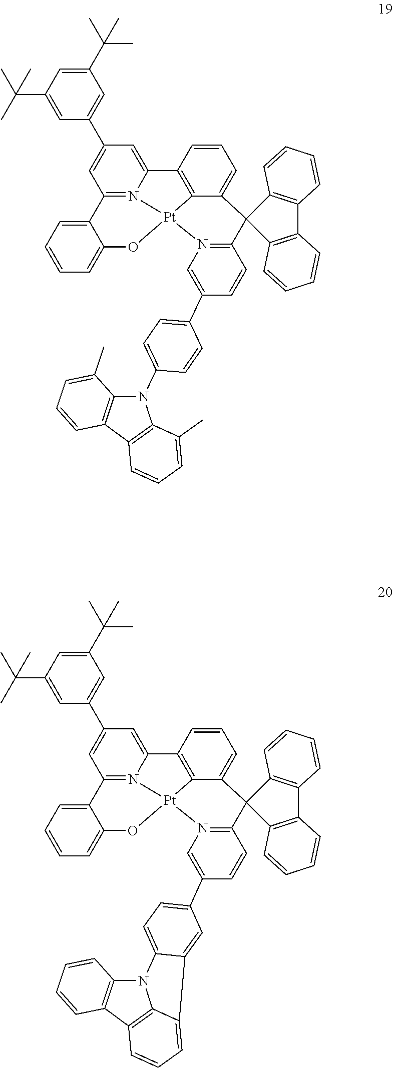

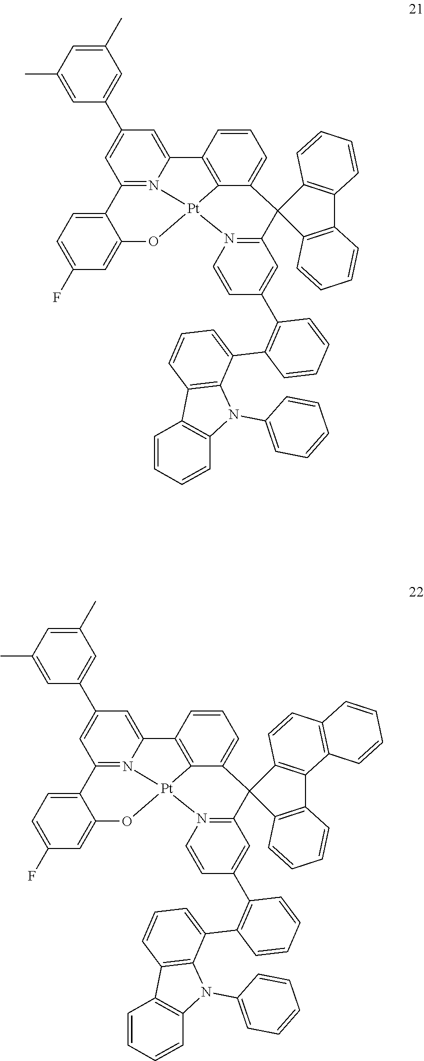

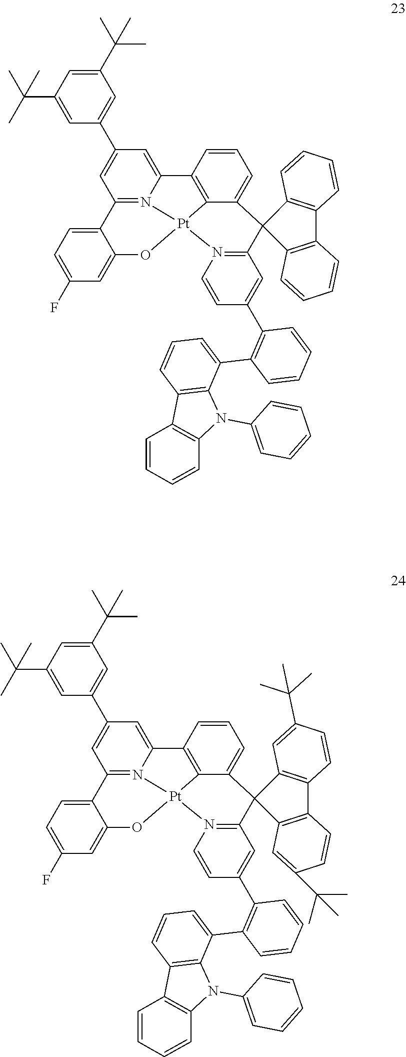

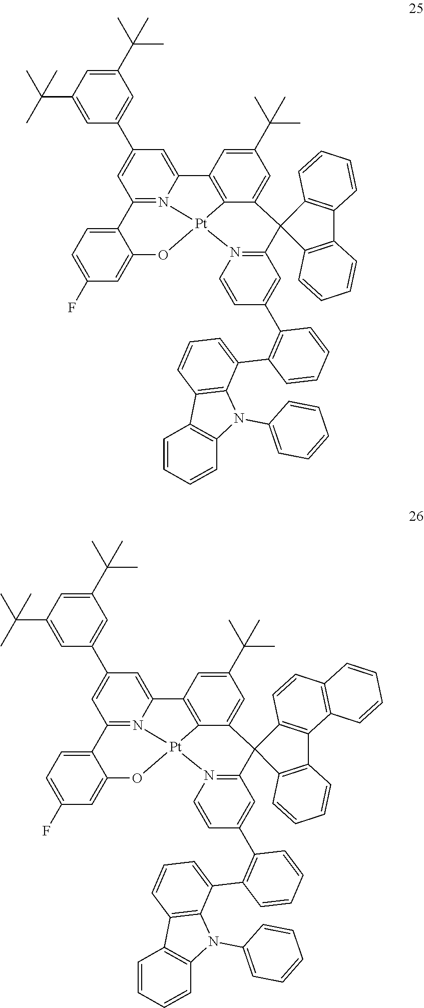

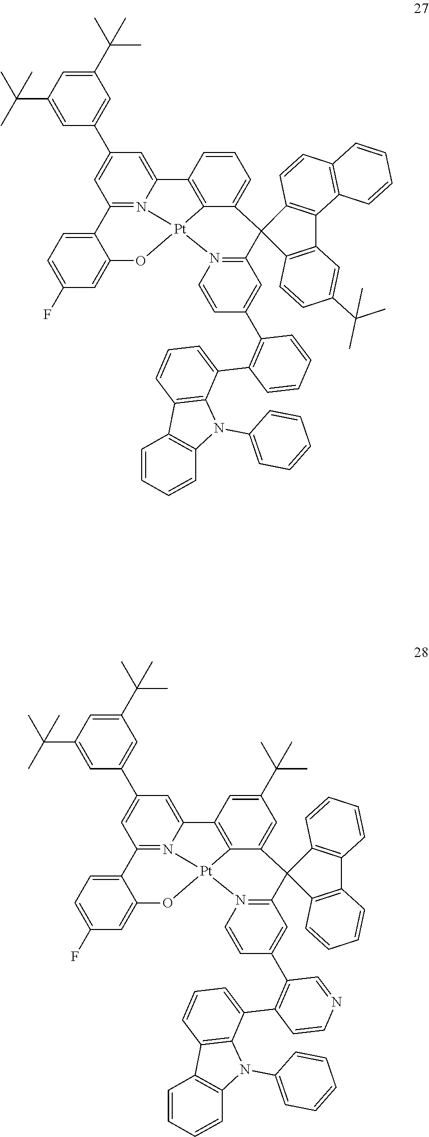

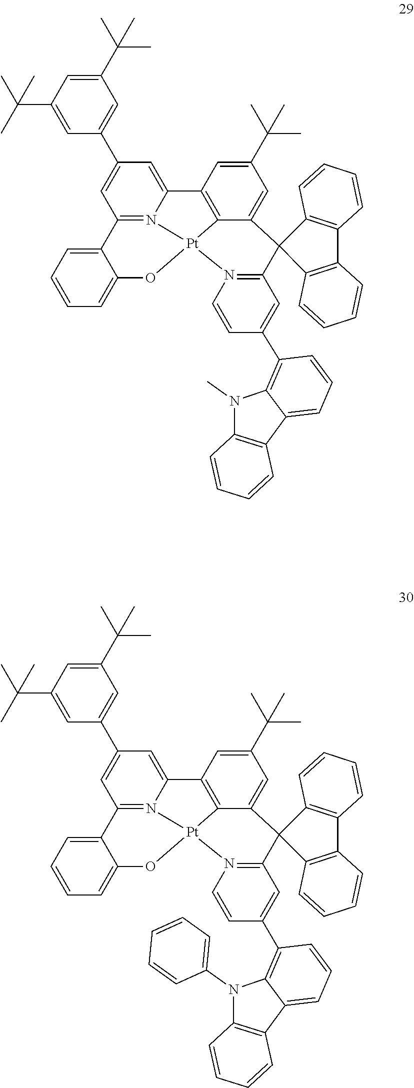

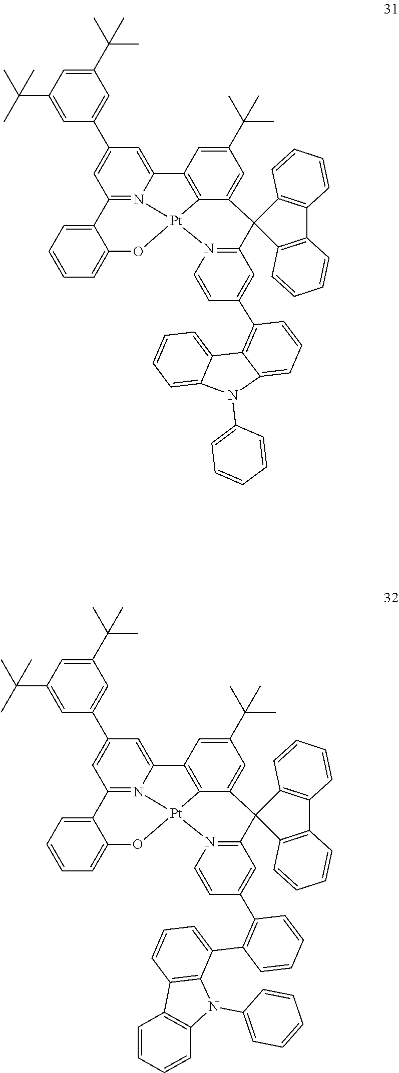

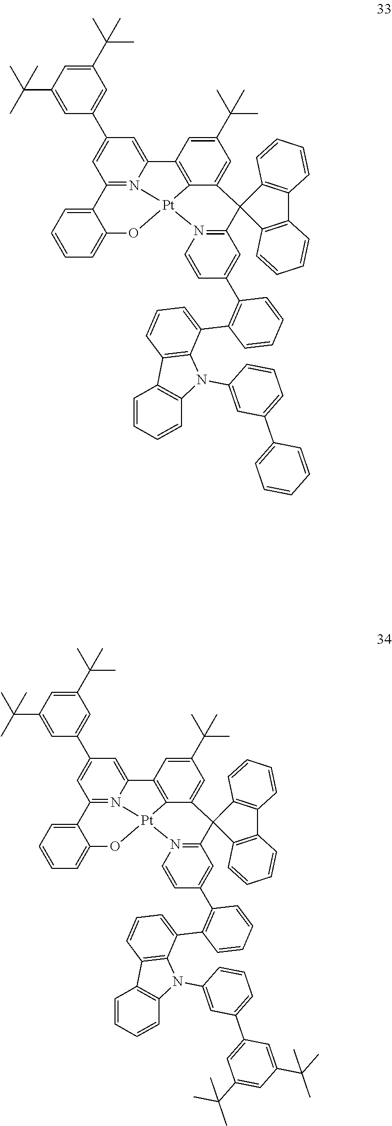

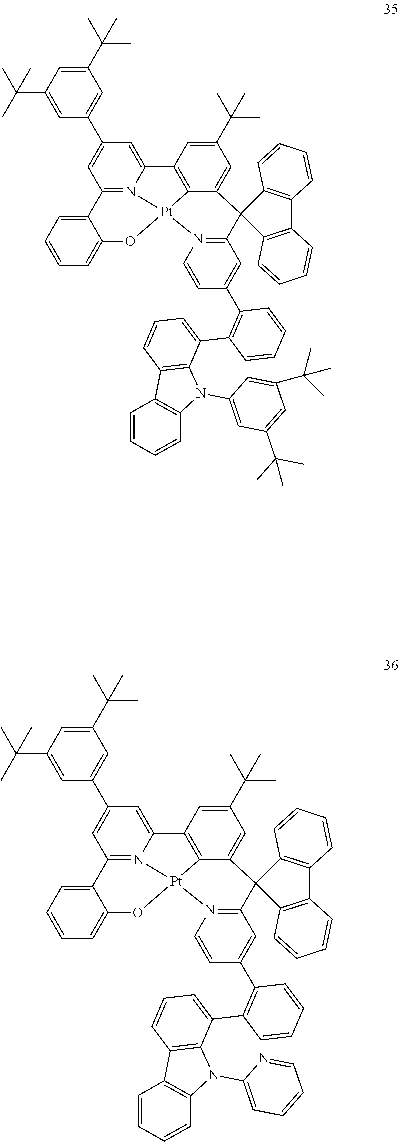

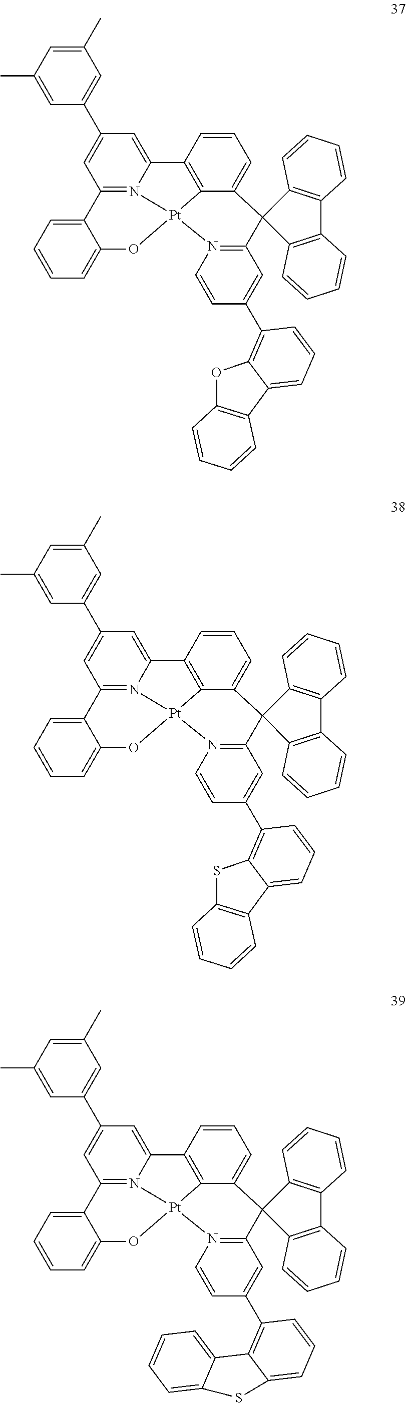

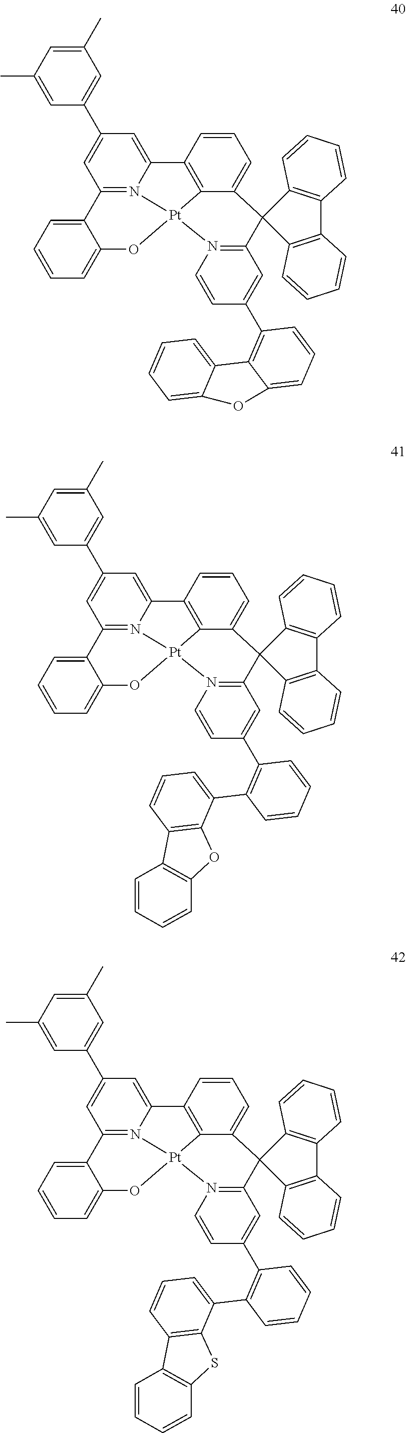

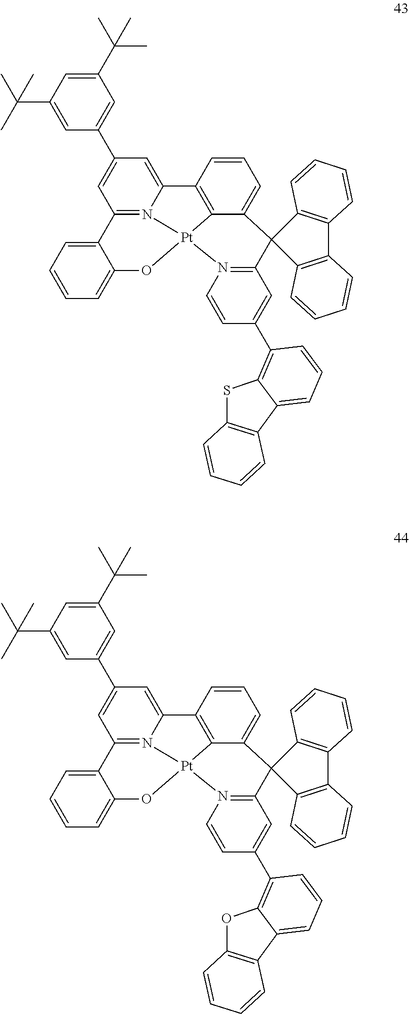

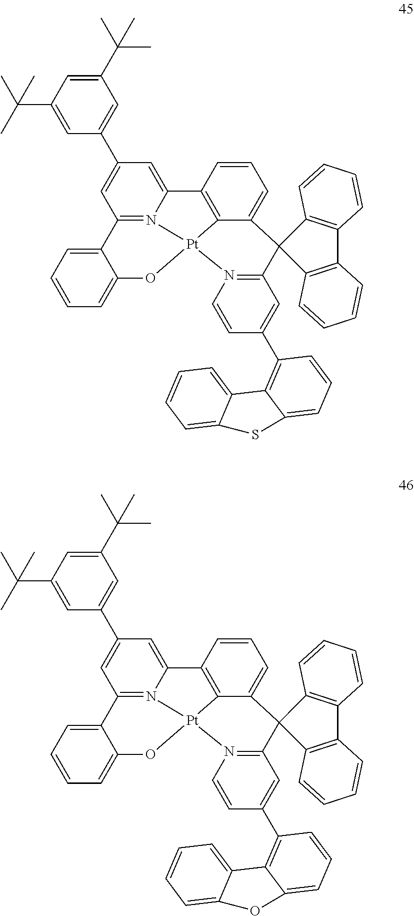

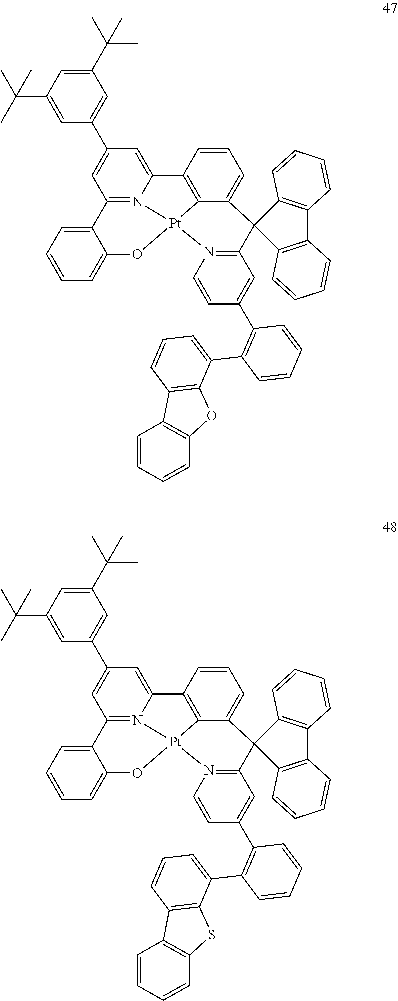

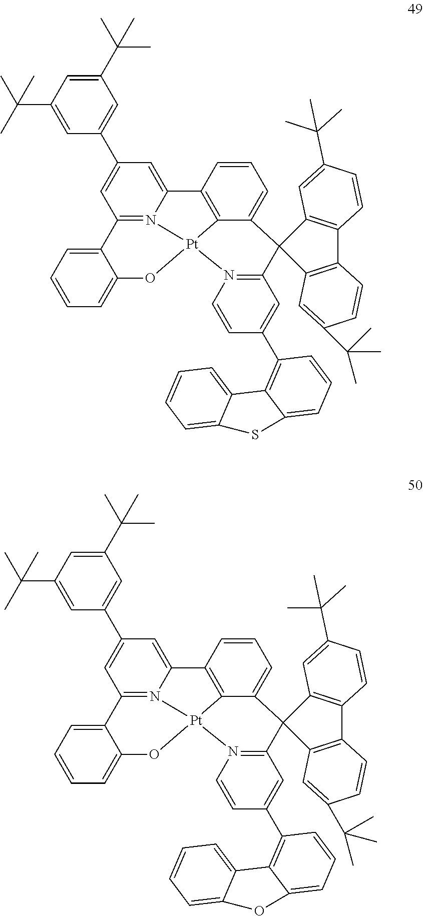

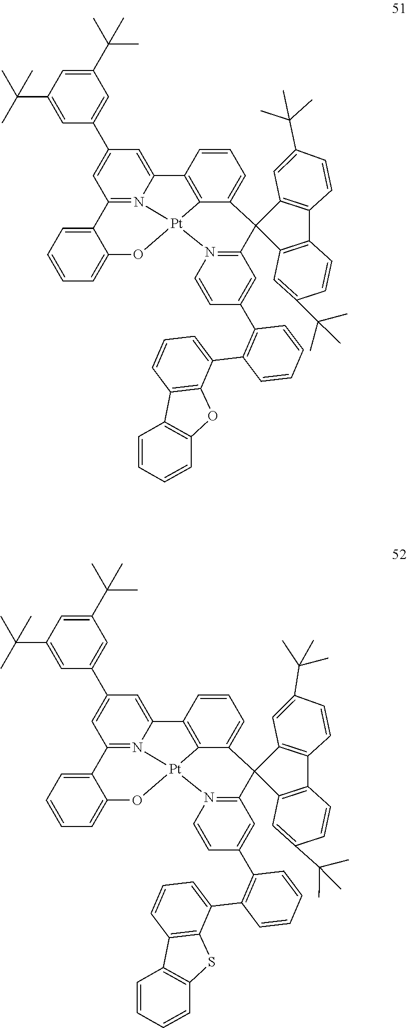

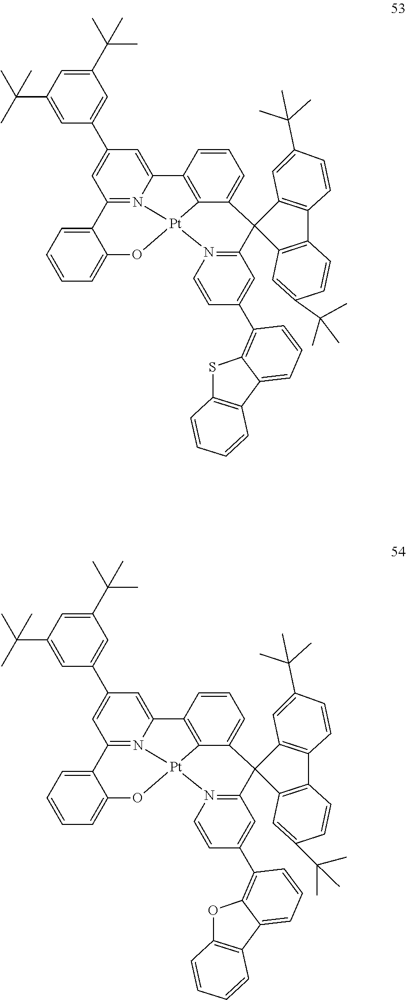

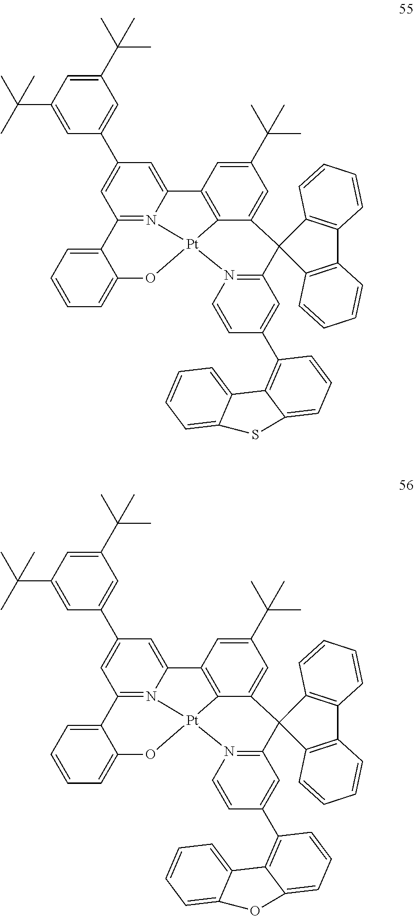

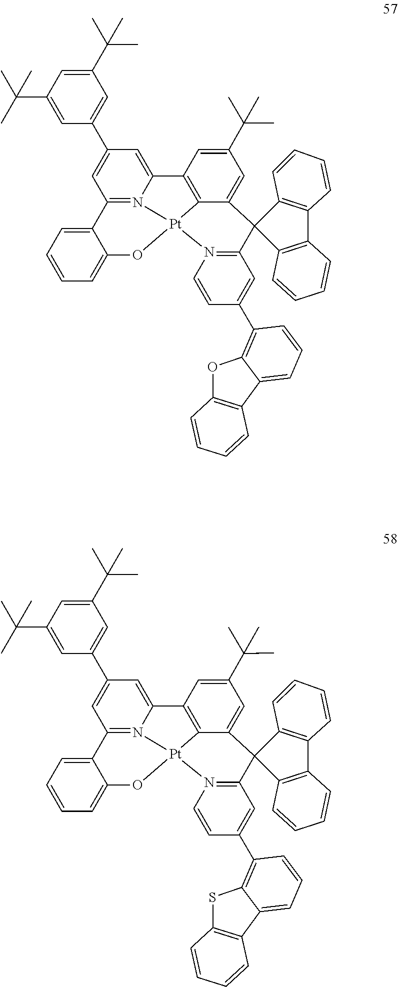

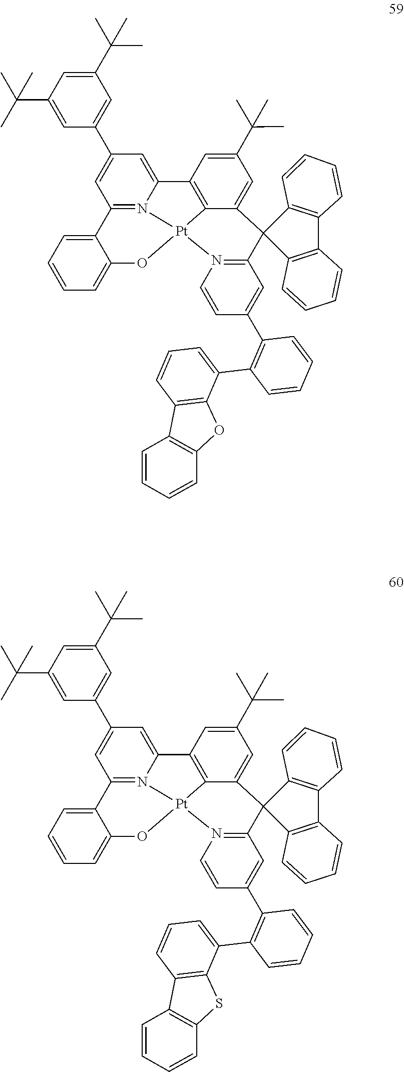

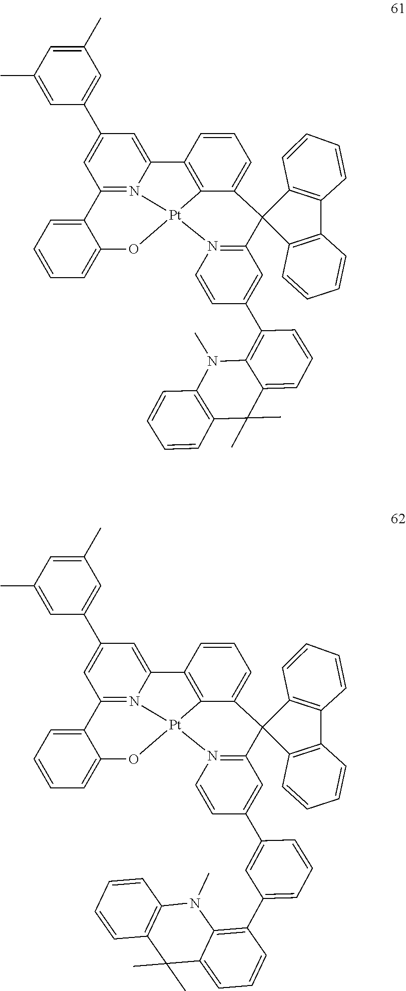

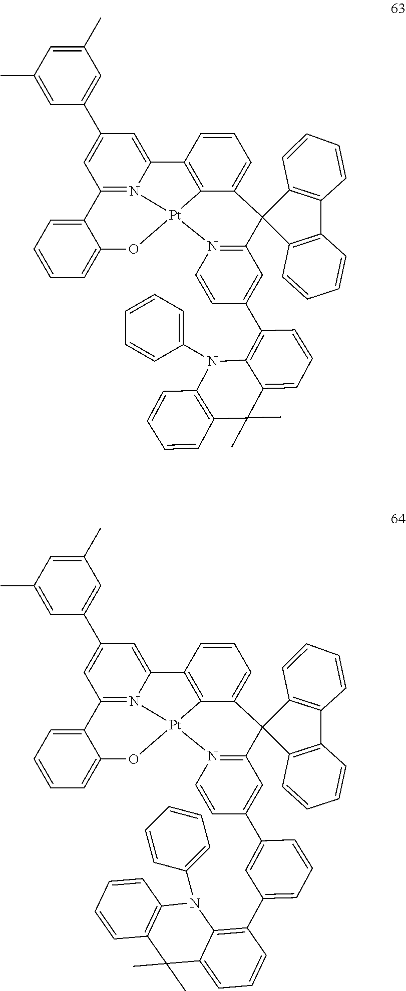

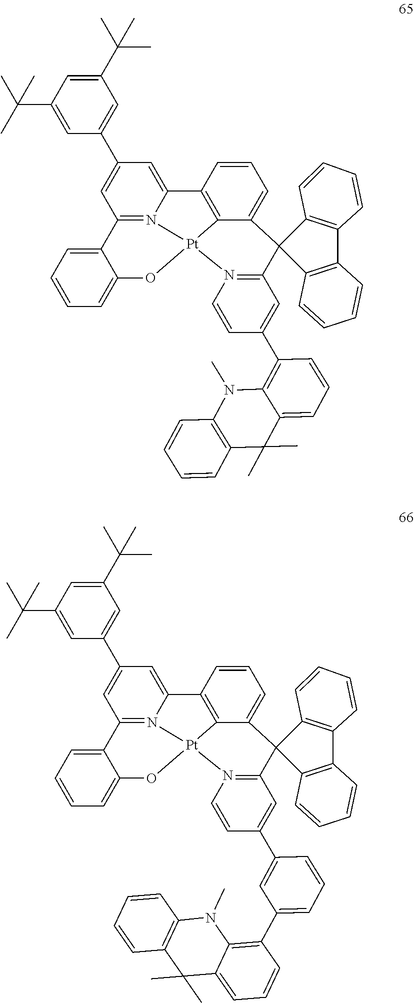

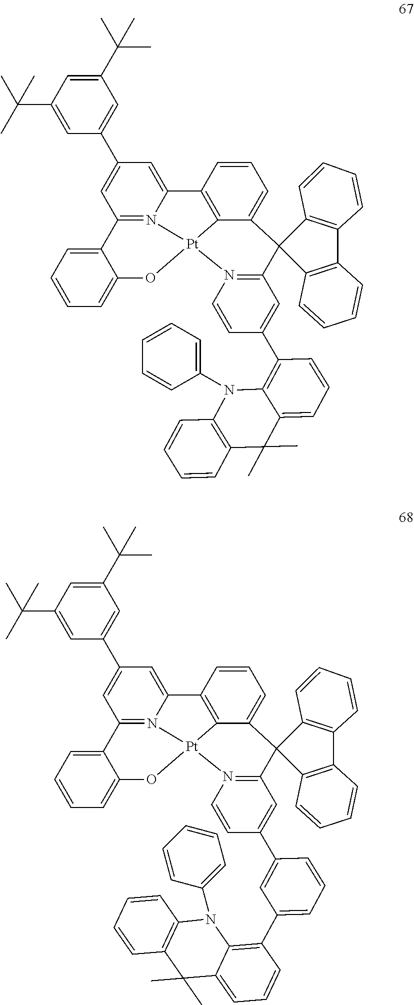

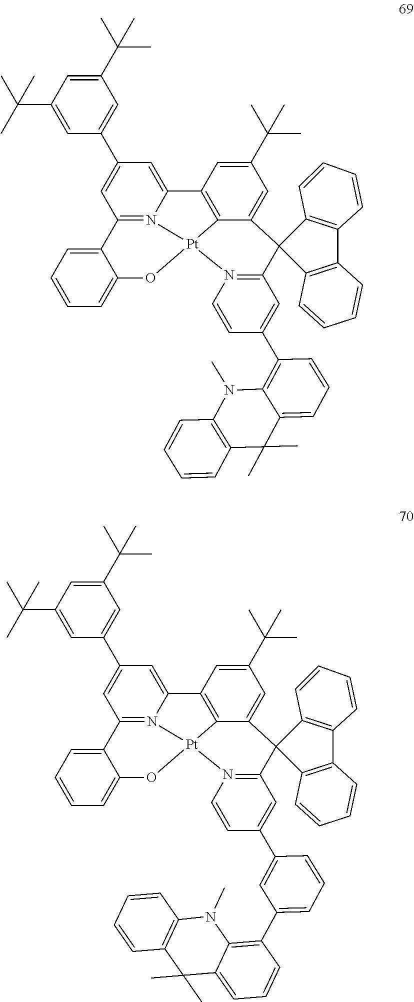

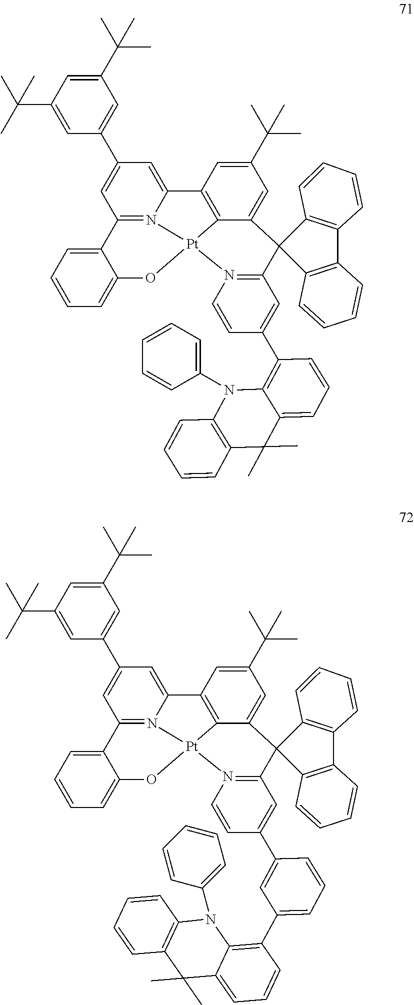

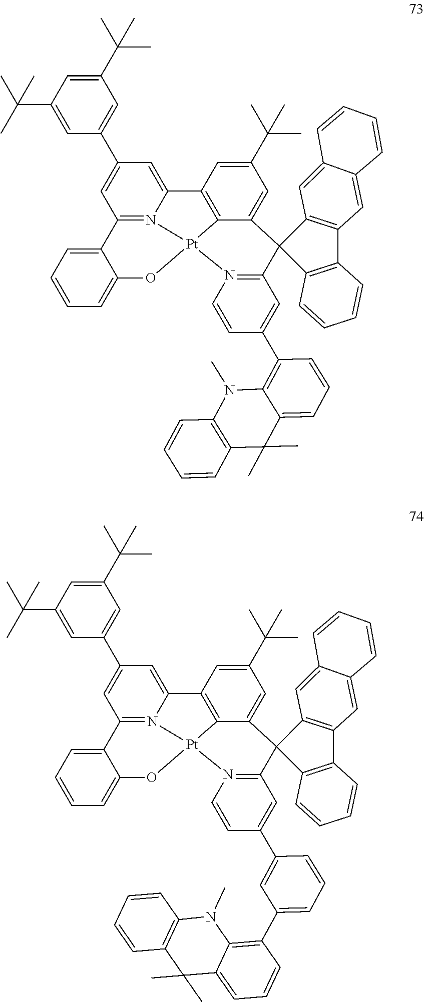

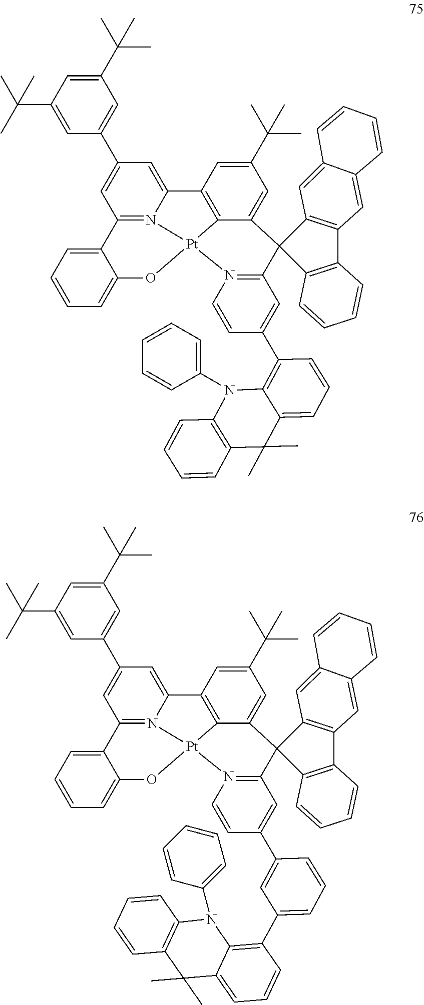

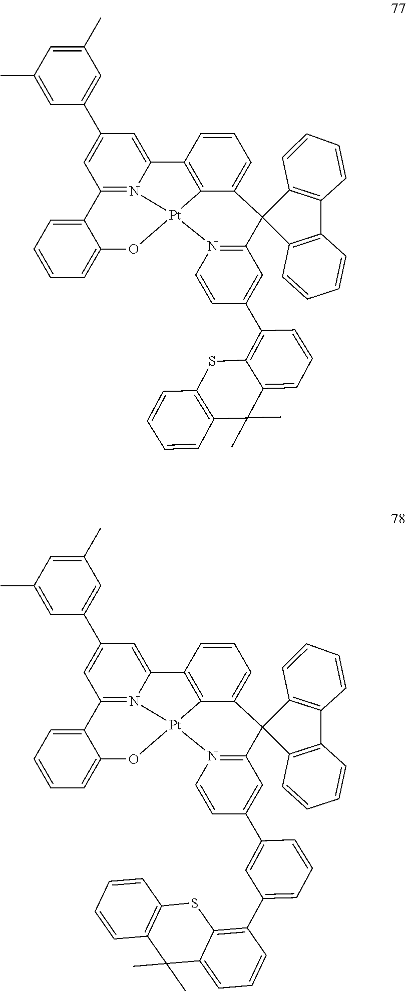

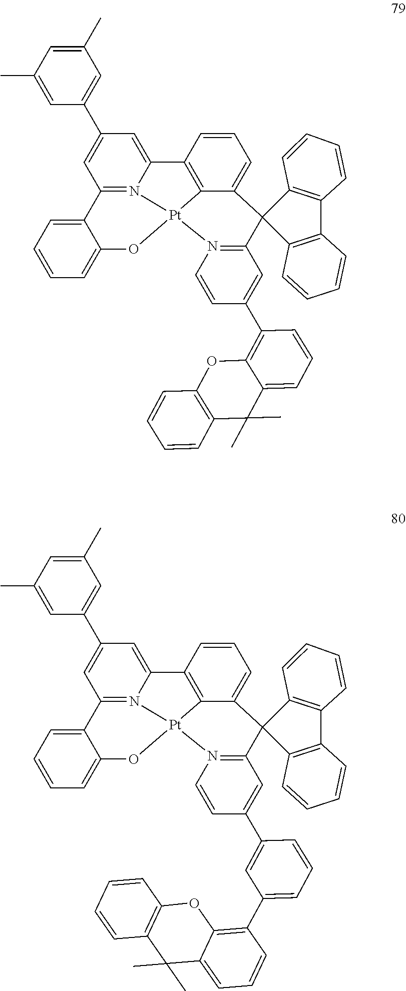

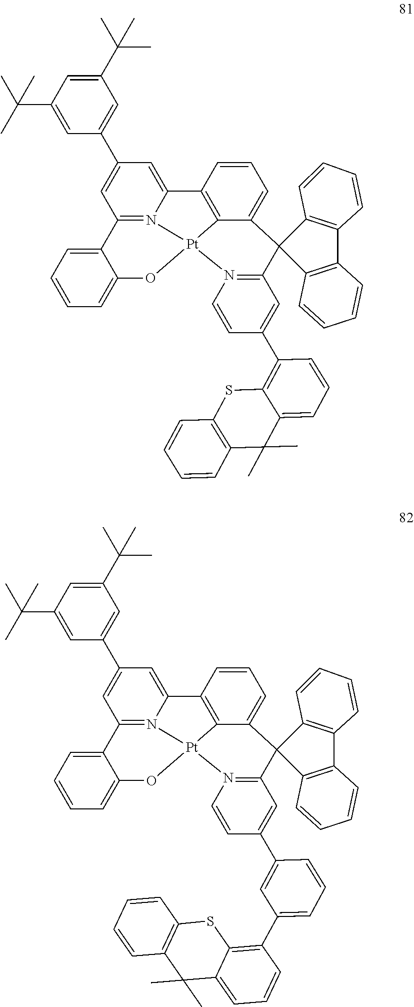

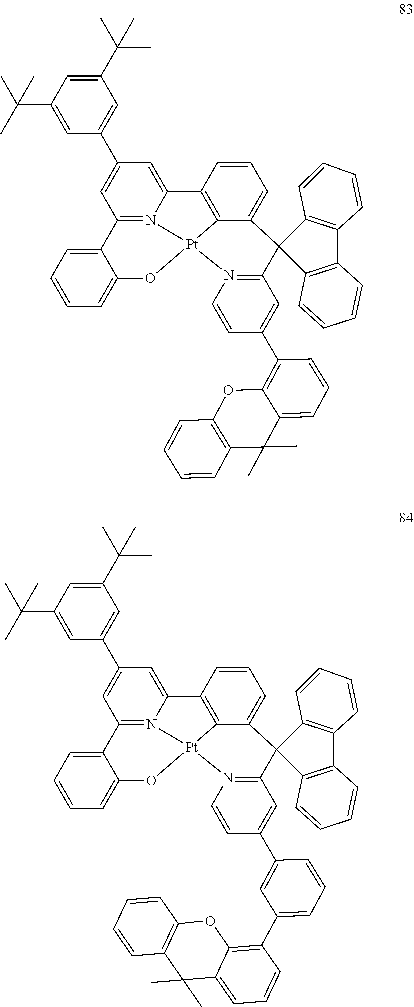

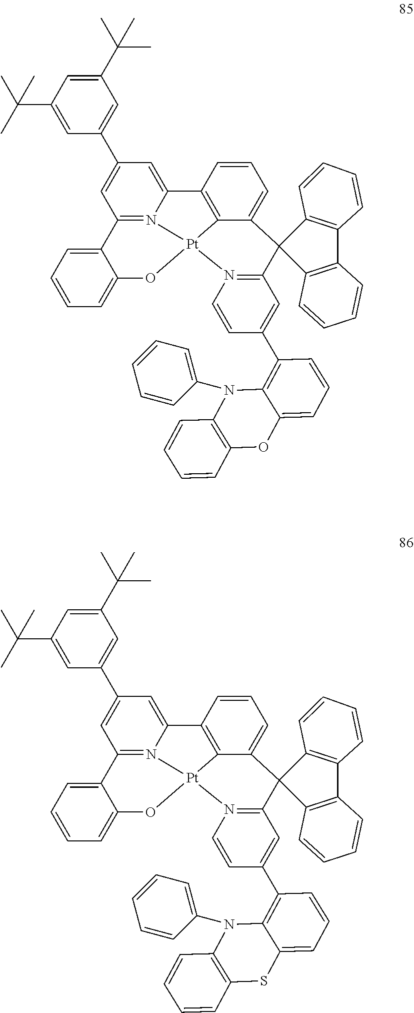

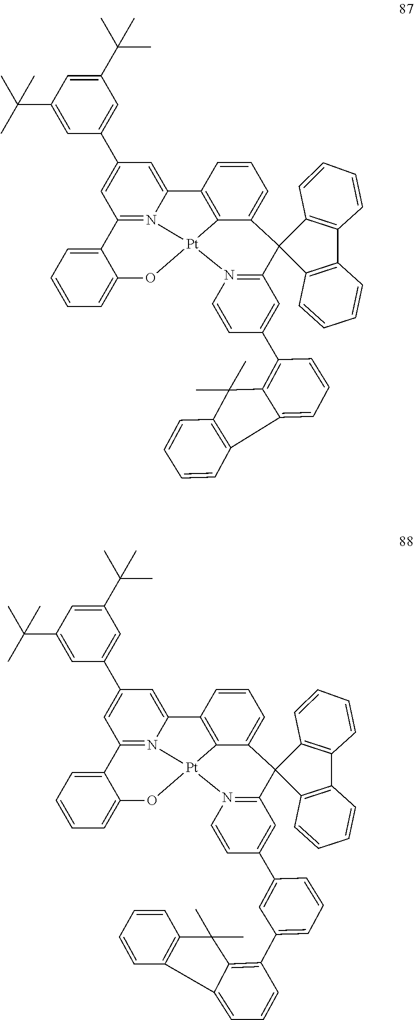

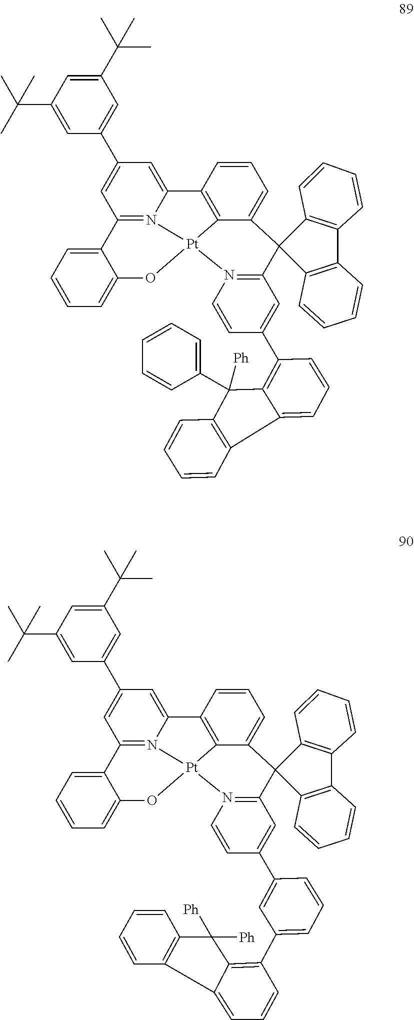

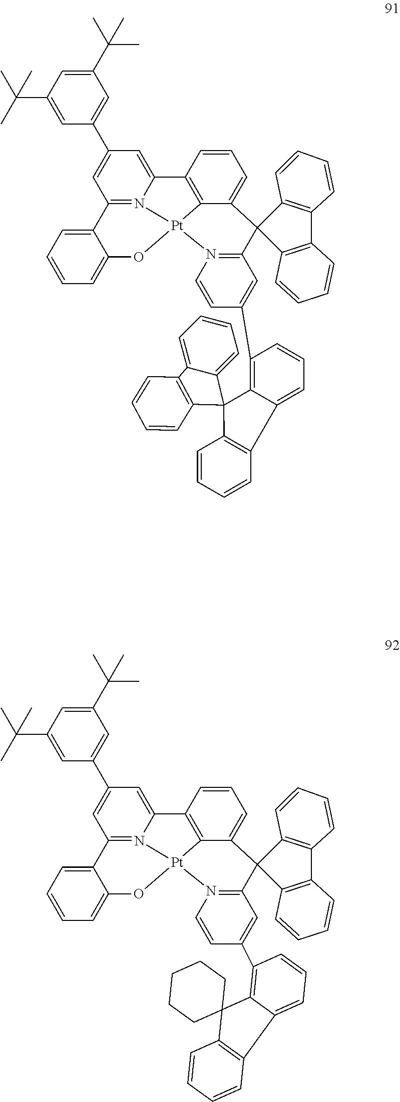

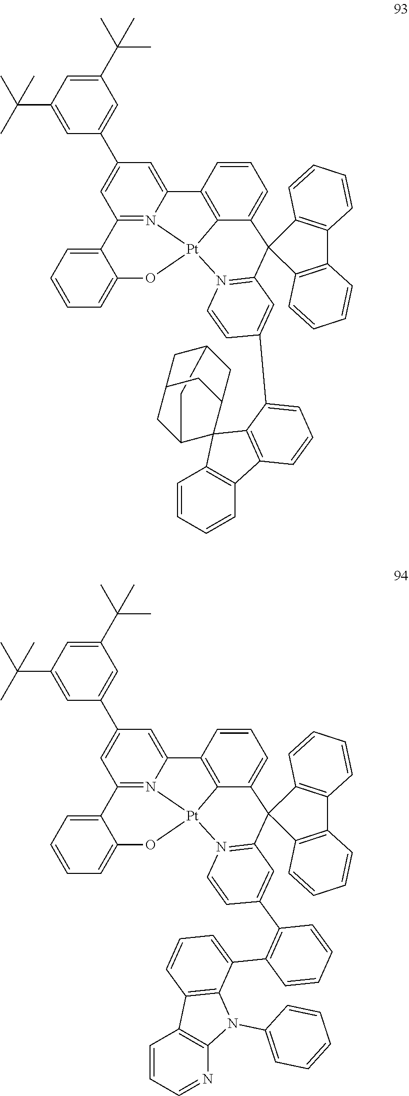

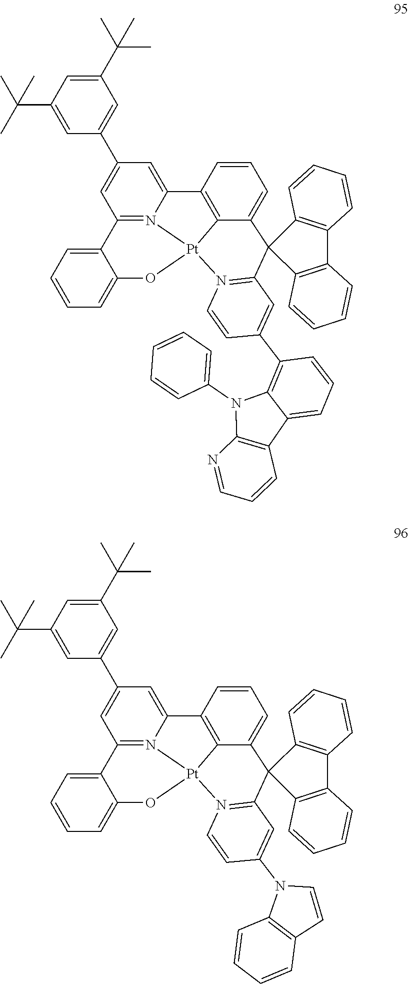

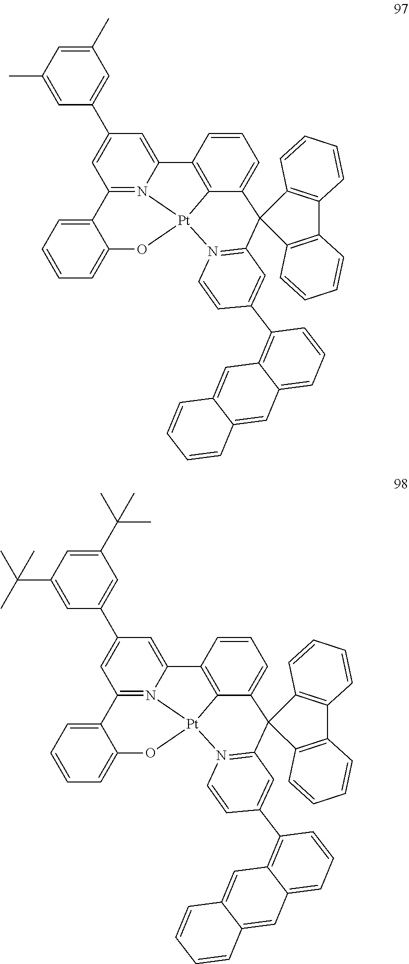

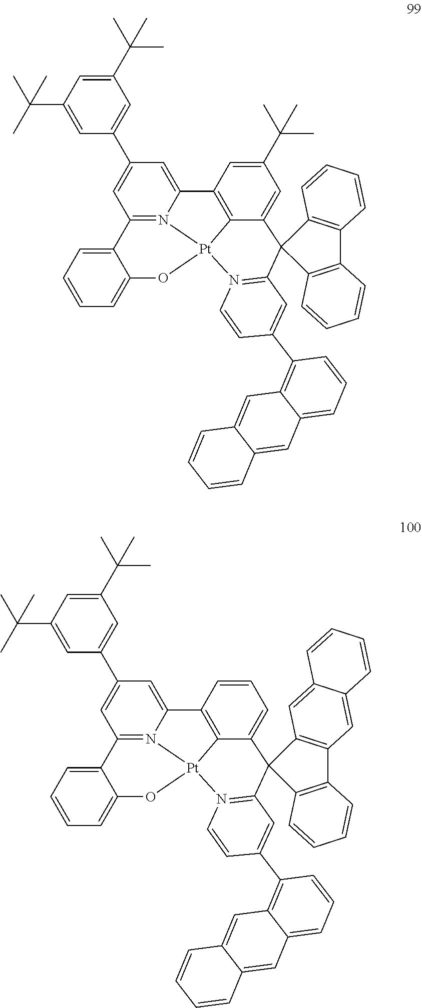

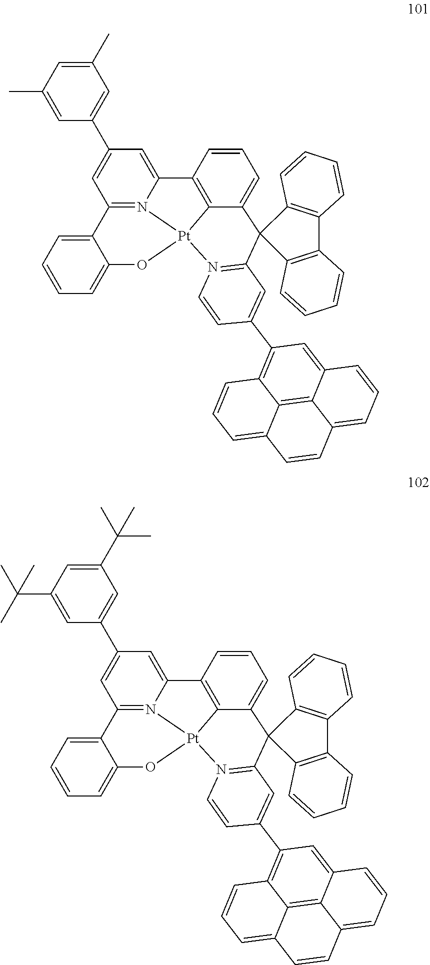

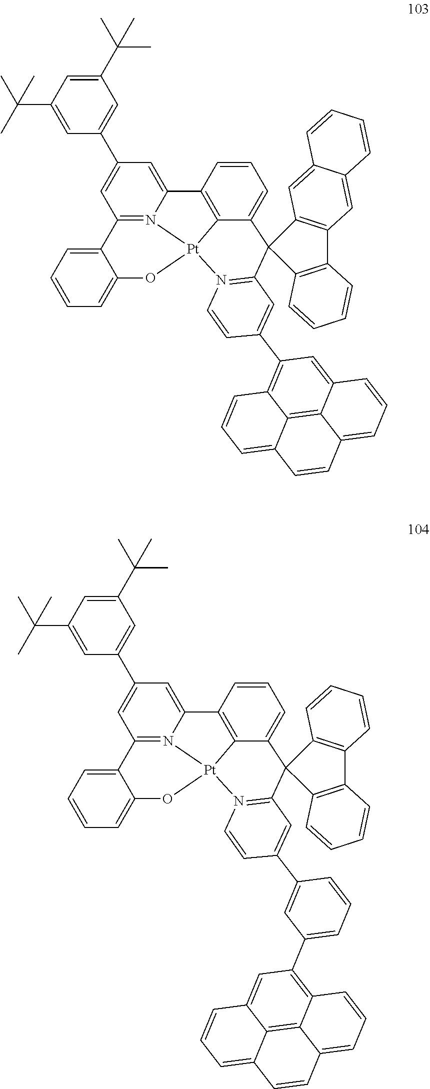

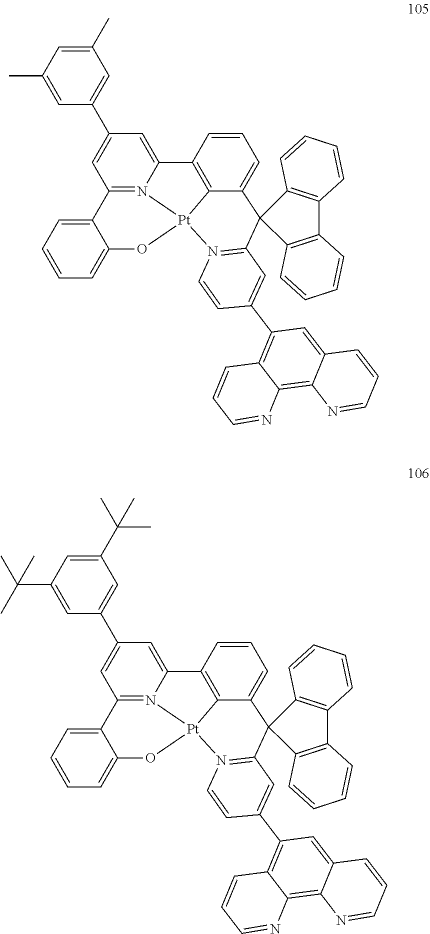

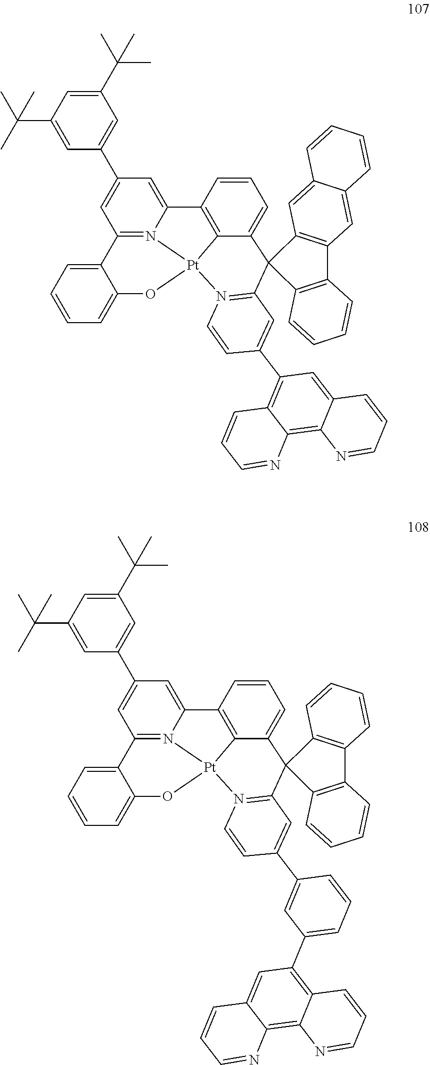

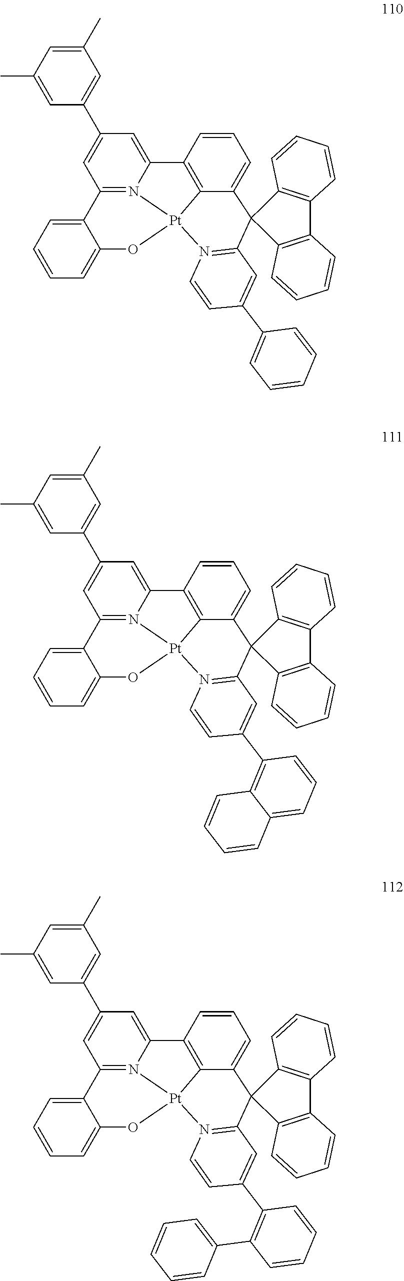

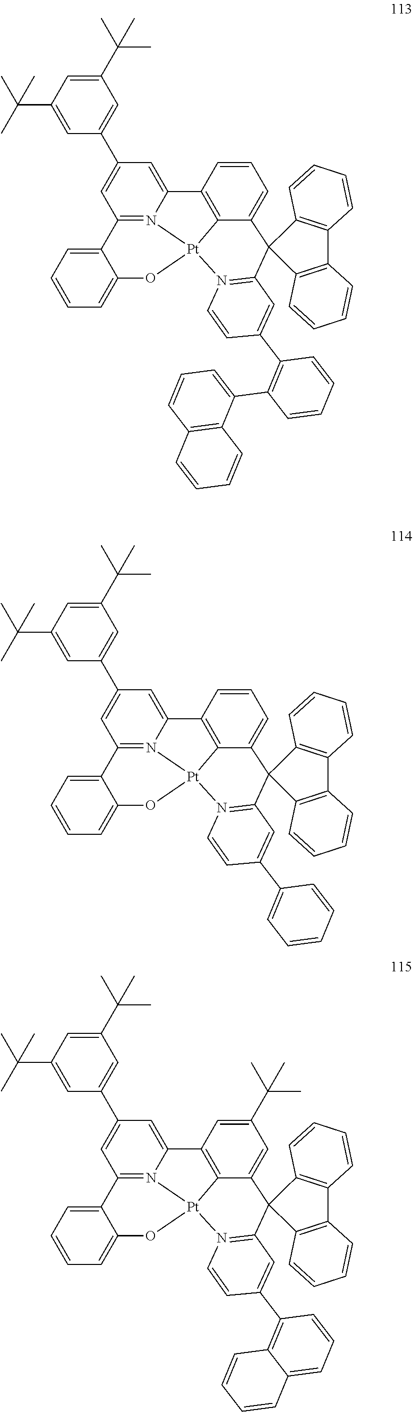

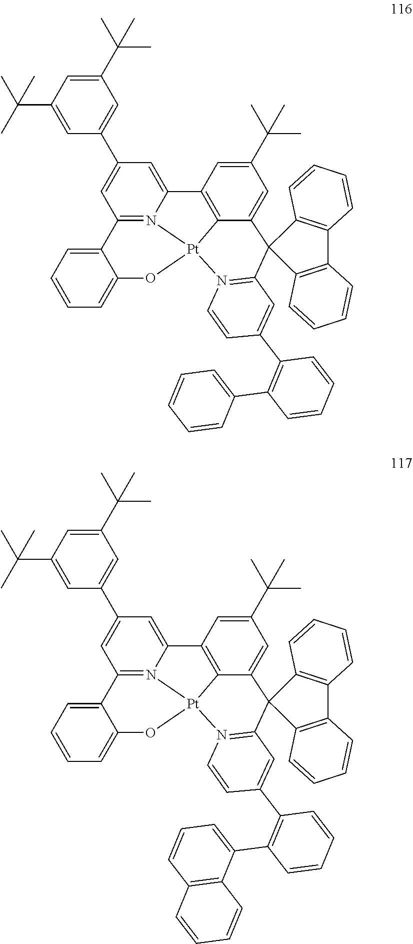

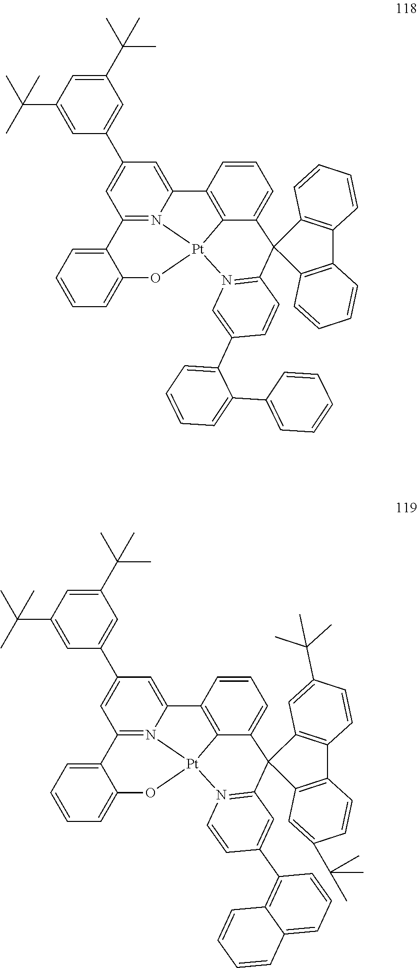

A precursor of the above compound has a structure of the following formula (III):

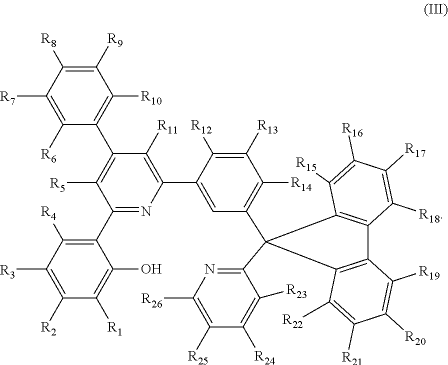

In some embodiments, the precursor has a structure of the following formula (IV):

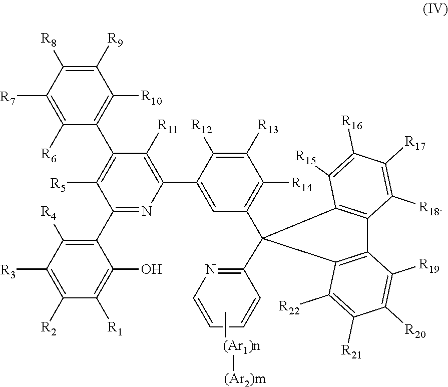

Provided is an organic light-emitting device including a light-emitting layer, where the light-emitting layer includes the above-mentioned platinum compound.

Provided is use of the platinum compound according to the present disclosure as a phosphorescent dopant material in a light-emitting layer of OLED.

The present disclosure improves material stability, device efficiency and service lifetime through structural design.

The introduction of spirofluorene in these compounds optimizes the molecular spatial configuration, and improves color purity and luminous efficiency.

This type of structure has multiple modifiable sites. In some embodiments, the increased steric hindrance from Ar₁ and Ar₂ groups contained in the structure can effectively reduce intermolecular aggregation.

The platinum compounds of the present disclosure exhibit high photoluminescence quantum yields, excellent thermal stability and low quenching constants, enabling the fabrication of highly efficient green-emitting OLED devices.

### BRIEF DESCRIPTION OF THE DRAWING

FIGURE is a structural diagram of the device according to the present disclosure.

### DETAILED DESCRIPTION

The present disclosure will be further described below in conjunction with the accompanying drawing and examples.

### Example 1: Synthesis of Intermediate M

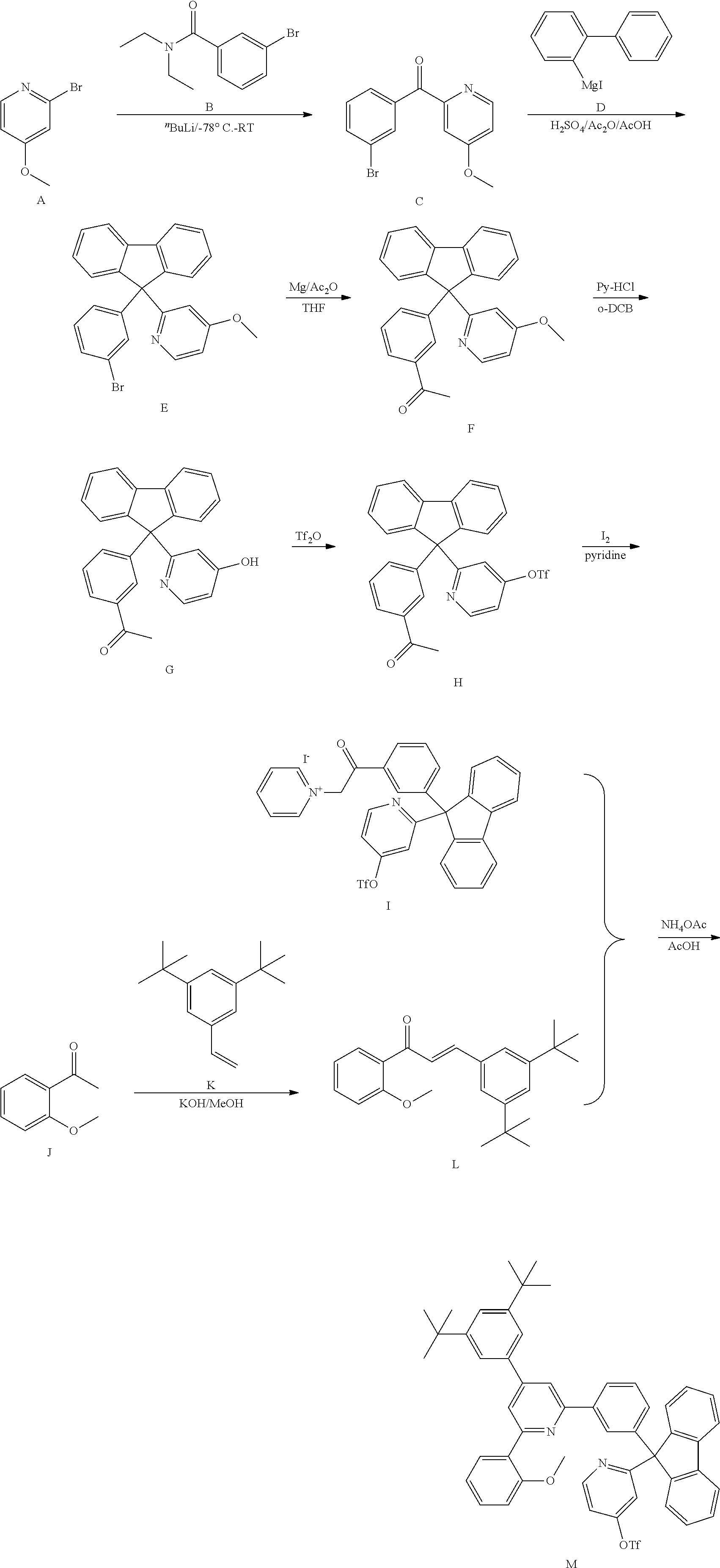

### Synthesis of Intermediate C

A 500 mL three-necked flask was charged with compound A (10.0 g, 53.5 mmol, 1.1 eq.), dissolved in diethyl ether (100 mL). Under nitrogen atmosphere, the solution was cooled to-78° C. To this cooled solution, 1.6 M n-butyllithium in n-hexane (33.4 mL) were added dropwise, resulting in a mixture, and the mixture was stirred for 30 min. Subsequently, the reaction solution was added dropwise to a solution of compound B (12.4 g) in diethyl ether (100 mL) at −78° C. The mixture was stirred at room temperature overnight, and then quenched with water. The mixture was extracted three times with ethyl acetate, and the combined organic layers were washed with saturated brine, dried over anhydrous sodium sulfate, and then concentrated under reduced pressure. The residue was purified by silica gel column chromatography (elution solvent:n-hexane/ethyl acetate=10:1 (V/V)) to obtain 11.2 g of a yellow oil-like substance, with a yield of 80%.

### Synthesis of Intermediate E

A 500 mL three-necked flask was charged with intermediate C (11.2 g, 38.6 mmol, 1.0 eq.), dissolved in anhydrous THF (100 mL). Under nitrogen atmosphere, the solution was cooled to −78° C. To this cooled solution, compound D (38.6 mmol) was added dropwise, resulting in a mixture, and the mixture was stirred at room temperature for 24 h. The reaction solution was quenched with saturated ammonium chloride solution, then extracted three times with ethyl acetate. The combined organic layers were washed with saturated brine, dried over anhydrous sodium sulfate, and concentrated under reduced pressure. The residue was purified by silica gel column chromatography (elution solvent:hexanes/ethyl acetate=1:5 (V/V)) to obtain a yellow oil-like substance. The obtained yellow oil-like substance was dissolved in acetic acid (100 mL) in a 500 mL three-necked flask. Then concentrated sulfuric acid (2 mL) and acetic anhydride (2 mL) were added. The reaction solution was stirred at 130° C. for 12 h under nitrogen atmosphere, with reaction progress monitored by TLC until complete consumption of starting materials. After the reaction mixture was cooled to room temperature, the solvent was distilled off under reduced pressure, and the pH was adjusted to weakly basic with a saturated sodium carbonate solution. The mixture was extracted with ethyl acetate three times, and the combined organic layers were dried over anhydrous sodium sulfate, filtered and concentrated under reduced pressure. The obtained crude product was separated by silica gel column chromatography (elution solvent:n-hexane/ethyl acetate=20:1-10:1 (V/V)), to obtain 13.7 g of yellow solid, with a yield of 83.5%.

### Synthesis of Intermediate F

A 500 mL three-necked flask was charged with anhydrous THF (150 mL) and Mg (770.4 mg, 32.1 mmol, 1.0 eq) and heated to 50° C. under nitrogen atmosphere. A solution of intermediate E (13.7 g, 32.1 mmol, 1.0 eq) in THF (50 mL) was added over 30 min and stirred until complete dissolution of Mg was achieved. Then the temperature was lowered to −10° C., and a solution of acetic anhydride (6.6 g, 64.2 mmol, 2.0 eq) in THF (50 mL) was added dropwise over 1 h. The reaction mixture was allowed to warm to room temperature and stirred until completion was confirmed by. The reaction solution was quenched with a saturated ammonium chloride solution and extracted with ethyl acetate for three times. The combined organic layers were washed with saturated brine, dried over anhydrous sodium sulfate, filtered and concentrated under reduced pressure. The residue was purified by silica gel column chromatography (elution solvent:n-hexane/ethyl acetate=10:1, (V/V)) to afford 10.2 g of a white solid with a yield of 82%.

### Synthesis of Intermediate G

In a 500 mL single-necked flask, intermediate F (10.2 g, 26.0 mmol, 1.0 eq), pyridine hydrochloride (102 g, 10 times by weight relative to F) and o-dichlorobenzene (10 mL) were mixed and heated for reaction at 200° C. in an oil bath for 8 h under nitrogen atmosphere, and then cooled to room temperature. The reaction mixture was diluted with copious amounts of water and extracted with dichloromethane (DCM) for three times. The combined organic players were concentrated under reduced pressure, dry-loaded onto silica gel, and purified by column chromatography (elution solvent:n-hexane/ethyl acetate=5:1, (V/V)) to afford 9.3 g of a pale yellow solid with a yield of 95%.

### Synthesis of Intermediate H

In a 500 mL three-necked flask, intermediate G (9.3 g, 24.7 mmol, 1.0 eq) and pyridine (3.9 g, 49.4 mmol, 2.0 eq) were dissolved in DCM (100 mL) were mixed under nitrogen atmosphere. The solution was cooled in an ice bath, and trifluoromethanesulfonic anhydride (Tf₂O (8.4 g, 29.6 mmol, 1.2 eq) was added dropwise within 30 min. The reaction mixture was stirred overnight at room temperature. The mixture was washed three times with saturated brine, and the organic players were concentrated under reduced pressure, dry-loaded onto silica gel, and purified by column chromatography (elution solvent:n-hexane/ethyl acetate=10:1, (V/V)) to afford 11.7 g of a yellow solid with a yield of 93%.

### Synthesis of Intermediate I

A 500 mL single-necked flask was charged with intermediate H (11.7 g, 22.9 mmol, 1.0 eq), I₂ (6.9 g, 27.48 mmol, 1.2 eq) and pyridine (100 mL) under nitrogen atmosphere. The mixture was stirred at 130° C. for 16 h. After the reaction was completed, the reaction mixture was directly filtered, and solids were triturated with methanol twice to afford 14.6 g of an off-white solid, with a yield of 89%.

### Synthesis of Intermediate L

A 500 mL single-necked flask was charged with compound J (5 g, 33.3 mmol, 1.0 eq), compound K (8.7 g, 40 mmol, 1.05 eq) and KOH (9.3 g, 166.5 mmol, 5.0 eq), dissolved in CH₃OH (100 mL). The system was purged with nitrogen, and the reaction was stirred at 45° C. for 16 h. After the reaction was completed, the mixture was filtered to collect a yellow solid, which was then triturated with n-hexane (50 mL) at 60° C. for 2 h, filtered again and dried to afford 8.7 g of a white solid with a yield of 80%.

### Synthesis of Intermediate M

A 500 mL single-necked flask was charged with intermediate L (6.5 g, 18.5 mmol, 1.0 eq), intermediate I (14.6 g, 20.4 mmol, 1.1 eq), NH₄OAc (65 g, 10 times by weight relative to L) and AcOH (130 mL) under nitrogen atmosphere. The reaction mixture was heated to 130° C. and stirred for 16 h. After completion, the mixture was cooled to room temperature, diluted with water (300 mL), and filtered to obtain a yellow solid. The crude product was purified by flash silica gel chromatography (eluting agent: n-hexane/ethyl acetate=10:1, (V/V)), to afford 14.2 g of a white solid, with a yield of 81%.

### Synthesis of Compound 114

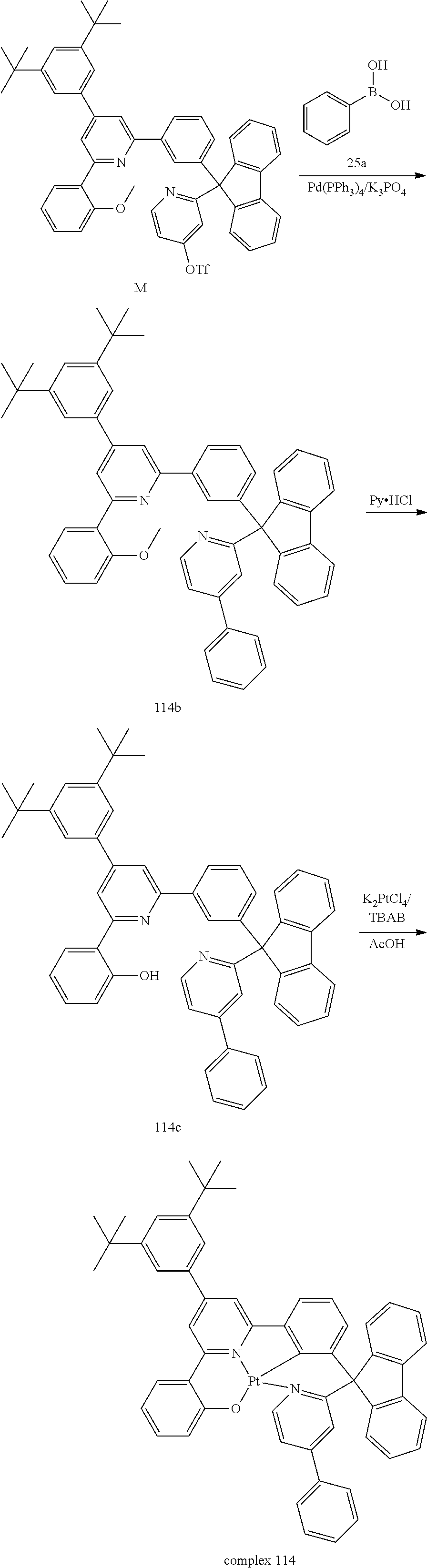

### Synthesis of Intermediate 114b

A 250 mL single-necked flask was charged with intermediate M (5 g, 5.96 mmol, 1.0 eq), compound 25a (872 mg, 7.15 mmol, 1.2 eq), Pd(PPh₃)₄ (344 mg, 0.298 mmol, 0.05 eq) and K₃PO₄ (2.53 g, 11.92 mmol, 2 eq) in a solvent mixture of toluene/EtOH/H₂O (30 mL/30 mL/15 mL). The system was purged with nitrogen, and the reaction was stirred at 80° C. for 16 h. After the reaction was completed, the mixture was filtered. The filtrate was concentrated in vacuo, and the residue was purified by flash silica gel chromatography (eluting agent: n-hexane/ethyl acetate=10:1, (V/V)), to afford 3.88 g of a white solid, with a yield of 85%.

### Synthesis of Intermediate 114c

A 100 mL single-necked flask was charged with 25b (3.88 g), pyridine hydrochloride (38.8 g) and o-dichlorobenzene (4.0 mL) under nitrogen protection. The mixture was heated at 200° C. in an oil bath for 8 h for reaction, and then cooled to room temperature. The reaction mixture was dissolved in copious amounts of water and extracted with DCM three times. The organic layers were concentrated under reduced pressure, dry-loaded onto silica gel, and purified by column chromatography (elution solvent:n-hexane/ethyl acetate=10:1, (V/V)) to afford 3.5 g of a pale yellow product with a yield of 92%.

### Synthesis of Compound 114

A 250 mL single-necked flask was charged with 2c (100 mg, 0.116 mmol), K₂PtCl₄ (57 mg, 0.139 mmol), tetrabutylammonium bromide (TBAB, 4 mg, 0.012 mmol) and acetic acid (5 mL) under nitrogen atmosphere. The mixture was stirred at 130° C. for 48 h. After the reaction was completed, excess deionized water was added to precipitate a solid, which was filtered and dissolved in dichloromethane, concentrated under reduced pressure, and dry-loaded onto silica gel for purification by flash chromatography (Hex:DCM:EA=20:20:1, V/V/V). The isolated product was recrystallized from DCM/n-Hexane=1:4, (V/V) to afford 80 mg of a red solid. ¹H NMR (500 MHz, Chloroform-d) 8 8.58 (d, J=5.0 Hz, 1H), 7.99 (dd, J=7.5, 2.0 Hz, 1H), 7.88 (s, 2H), 7.74-7.69 (m, 3H), 7.67-7.63 (m, 2H), 7.62-7.58 (m, 2H), 7.49-7.35 (m, 13H), 7.32 (d, J=2.0 Hz, 2H), 7.23 (td, J=7.5, 2.0 Hz, 1H), 7.19 (dd, J=7.4, 2.0 Hz, 1H), 1.36 (s, 18H).

### Example 2: Synthesis of Compound 9

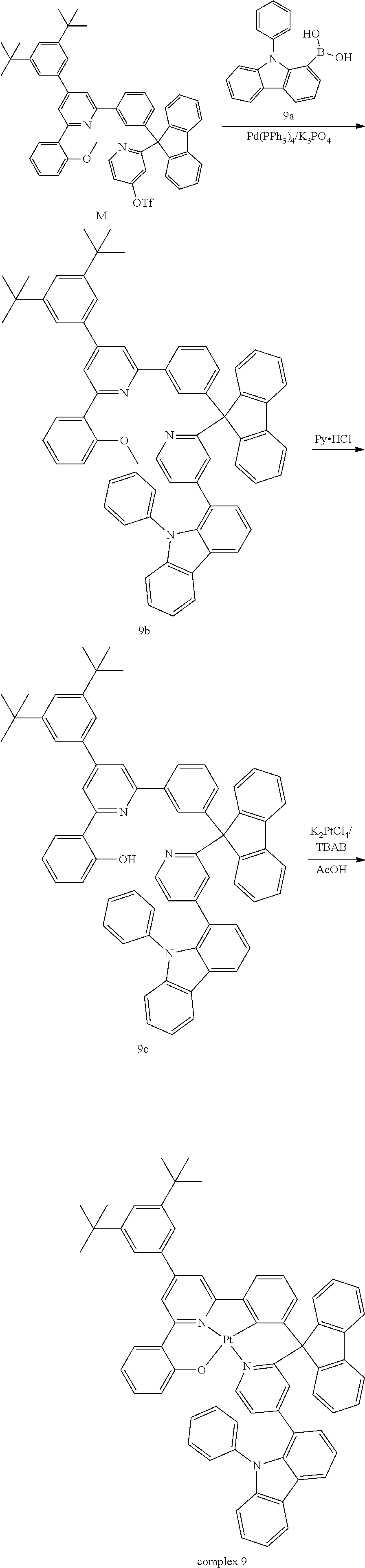

### Synthesis of Intermediate 9b

A 250 mL single-necked flask was charged with intermediate M (5 g, 5.96 mmol, 1.0 eq), compound 9a (1.79 g, 7.15 mmol, 1.2 eq), Pd(PPh₃)₄ (304 mg, 0.26 mmol, 0.05 eq) and K₃PO₄ (2.23 g, 10.52 mmol, 2 eq), dissolved in a solvent mixture of Tol/EtOH/H₂O (30 mL/30 mL/15 mL). The system was purged with nitrogen, and the reaction mixture was stirred at 80° C. for 16 h. After the reaction was completed, the mixture was filtered. The filtrate was concentrated under reduced pressure, and the residue was purified by flash silica gel column chromatography (eluting agent: n-hexane/ethyl acetate=10:1 (V/V)) to afford 4.78 g of a white solid with a yield of 87%.

A 100 mL single-necked flask was charged with intermediate 9b (4.78 g), pyridine hydrochloride (37.8 g) and o-dichlorobenzene (5 mL) under nitrogen atmosphere. The mixture was heated in an oil bath at 200° C. for 8 h for reaction, and then cooled to room temperature. The reaction mixture was dissolved with copious amounts of water and extracted with DCM three times. The organic layers were concentrated under reduced pressure, dry-loaded onto silica gel, and purified by column chromatography (elution solvent:n-hexane:ethyl acetate=10:1, (V/V)) to afford 4.37 g of a pale yellow product with a yield of 93%.

### Synthesis of Compound 9

A 250 mL single-necked flask was charged with intermediate 9c (100 mg, 0.11 mmol), K₂PtCl₄ (57 mg, 0.139 mmol), TBAB (4 mg, 0.011 mmol) and acetic acid (5 mL) under nitrogen atmosphere, and the mixture was reacted at 130 80° C. for 48 h. After the reaction was completed, excess deionized water was added to precipitate the solid, which was filtered and dissolved in dichloromethane, concentrated under reduced pressure, dry-loaded onto silica gel, and purified by column chromatography (Hex:DCM:EA=20:20:1, V/V/V). The collected product was further recrystallized from DCM:hexane=1:4 (V/V) to afford 90 mg of a red solid.

¹H NMR (500 MHz, Chloroform-d) δ 8.47 (d, J=4.9 Hz, 1H), 8.20 (s, 1H), 8.13 (s, 1H), 7.99 (dd, J=7.5, 2.0 Hz, 1H), 7.88 (s, 2H), 7.79 (s, 1H), 7.75-7.70 (m, 2H), 7.70-7.62 (m, 4H), 7.57 (s, 1H), 7.50-7.37 (m, 16H), 7.35 (s, 1H), 7.32 (d, J=2.0 Hz, 2H), 7.26 (s, 1H), 7.23 (td, J=7.5, 2.0 Hz, 1H), 7.19 (dd, J=7.4, 2.0 Hz, 1H), 1.36 (s, 18H).

### Example 3: Synthesis of Compound 5

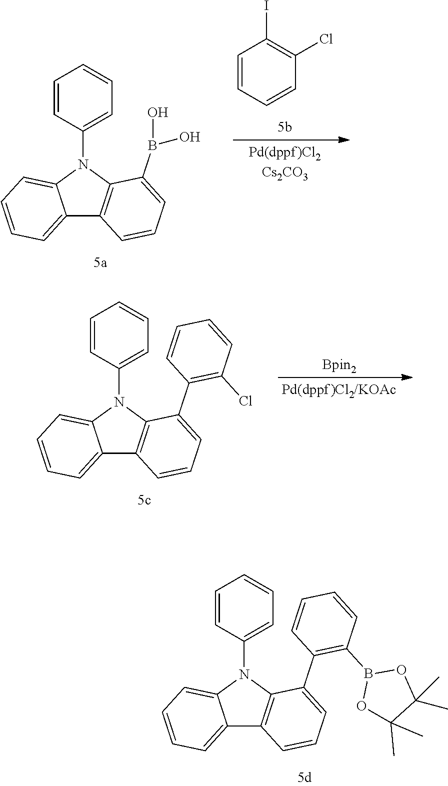 [SMILES: OB(O)c1cccc2c3ccccc3n(-c3ccccc3)c12 ; Clc1ccccc1I ; Clc1ccccc1-c1cccc2c3ccccc3n(-c3ccccc3)c12 ; CC1(C)OB(c2ccccc2-c2cccc3c4ccccc4n(-c4ccccc4)c23)OC1(C)C]

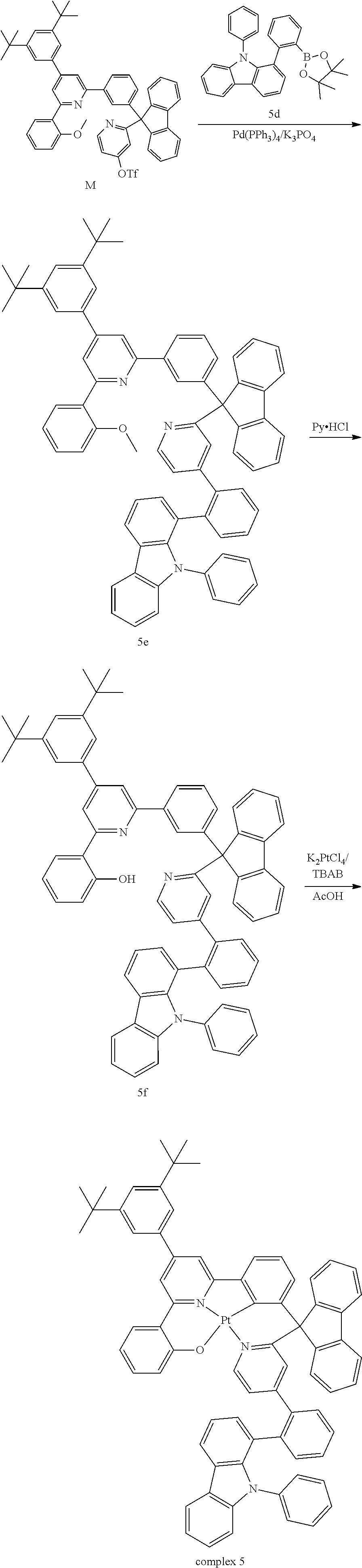

### Synthesis of Intermediate 5c

A 250 mL single-necked flask was charged with intermediate 5a (5 g, 17.4 mmol, 1.0 eq), 5b (4.14 g, 17.4 mmol, 1.0 eq), Pd(dppf)Cl₂ (635 mg, 0.87 mmol, 0.05 eq) and Cs₂CO₃ (11.3 g, 34.8 mmol, 2 eq), dissolved in dioxane (50 mL) under nitrogen atmosphere for reaction at 100° C. for 16 h. After the reaction was completed, the mixture was filtered, and the filtrate was concentrated under reduced pressure. The residue was purified by flash column chromatography on silica gel (eluting agent: n-hexane/ethyl acetate=10:1, (V/V)), to afford 10 4.88 g of a white solid, with a yield of 80%.

### Synthesis of Intermediate 5d

A 250 mL single-necked flask was charged with compound 5c (4.88 g, 13.8 mmol, 1.0 eq), Bpin₂ (5.25 g, 20.7 mmol, 1.5 eq), Pd(dppf)Cl₂ (504 mg, 0.69 mmol, 0.05 eq) and KOAc (2.7 g, 27.6 mmol, 2 eq), dissolved in toluene (50 mL) under nitrogen atmosphere for reaction at 100° C. for 16 h. After the reaction was completed, the mixture was filtered, and the filtrate was concentrated under reduced pressure. The residue was purified by flash column chromatography on silica gel (eluting agent: n-hexane/ethyl acetate=10:1, (V/V)) to afford 5.2 g of a white solid with a yield of 85%.

### Synthesis of Intermediate 5e

A 250 mL single-necked flask was charged with intermediate M (5 g, 5.96 mmol, 1.0 eq), intermediate 5d (2.77 g, 7.15 mmol, 1.2 eq), Pd(PPh₃)₄ (304 mg, 0.26 mmol, 0.05 eq) and K₃PO₄ (2.23 g, 10.52 mmol, 2 eq), dissolved in a solvent mixture of Tol/EtOH/H₂O (30 mL/30 mL/15 mL) under nitrogen atmosphere for reaction at 80° C. for 16 h. After the reaction was completed, the mixture was filtered, and the filtrate was concentrated under reduced pressure. The residue was purified by flash column chromatography on silica gel (eluting agent: n-hexane/ethyl acetate=10:1, (V/V)) to afford 4.8 g of a white solid with a yield of 80%.

### Synthesis of Intermediate 5f

A 100 mL single-necked flask was charged with intermediate 5e (4.8 g), pyridine hydrochloride (48 g) and o-dichlorobenzene (5 mL) under nitrogen atmosphere for reaction 200° C. in an oil bath for 8 h, and then cooled to room temperature. The reaction mixture was dissolved with copious amounts of water and extracted with DCM three times. The organic layers were concentrated under reduced pressure, dry-loaded onto silica gel, and purified by column chromatography (elution solvent:n-hexane:ethyl acetate=10:1, (V/V)) to afford 4.32 g of a pale yellow product with a yield of 92%.

### Synthesis of Compound 5

A 250 mL single-necked flask was charged with intermediate 5f (100 mg, 0.1 mmol), K₂PtCl₄ (45 mg, 0.12 mmol), TBAB (3 mg, 0.01 mmol) and acetic acid (5 mL) under nitrogen atmosphere for reaction at 130° C. for 48 h. After the reaction was completed, excess deionized water was added to precipitate the solid, which was filtered and dissolved in dichloromethane, concentrated under reduced pressure, dry-loaded onto silica gel, and purified by column chromatography (Hex:DCM:EA=20:20:1, V/V/V). The collected product was further recrystallized from DCM:hexane=1:4 (V/V) to afford 85 mg of a red solid.

¹H NMR (500 MHz, Chloroform-d) δ 8.47 (d, J=4.9 Hz, 1H), 8.20 (s, 1H), 8.13 (s, 1H), 7.99 (dd, J=7.5, 2.0 Hz, 1H), 7.88 (s, 2H), 7.85 (s, 1H), 7.75-7.70 (m, 2H), 7.69-7.67 (m, 2H), 7.66-7.62 (m, 3H), 7.62-7.58 (m, 2H), 7.56-7.36 (m, 17H), 7.35 (s, 1H), 7.32 (d, J=2.0 Hz, 2H), 7.26 (s, 1H), 7.23 (td, J=7.5, 2.0 Hz, 1H), 7.19 (dd, J=7.4, 2.0 Hz, 1H), 1.36 (s, 18H).

### Example 4: Synthesis of Compound 12

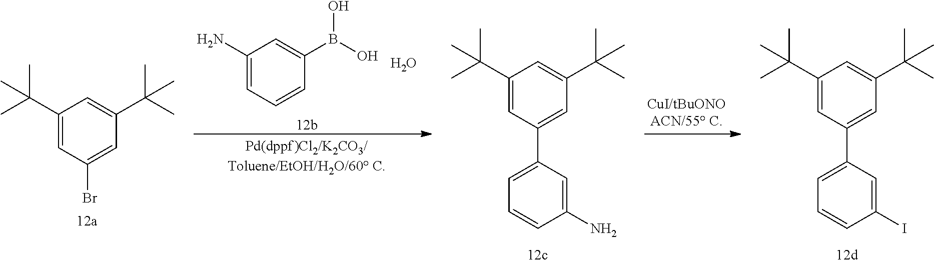

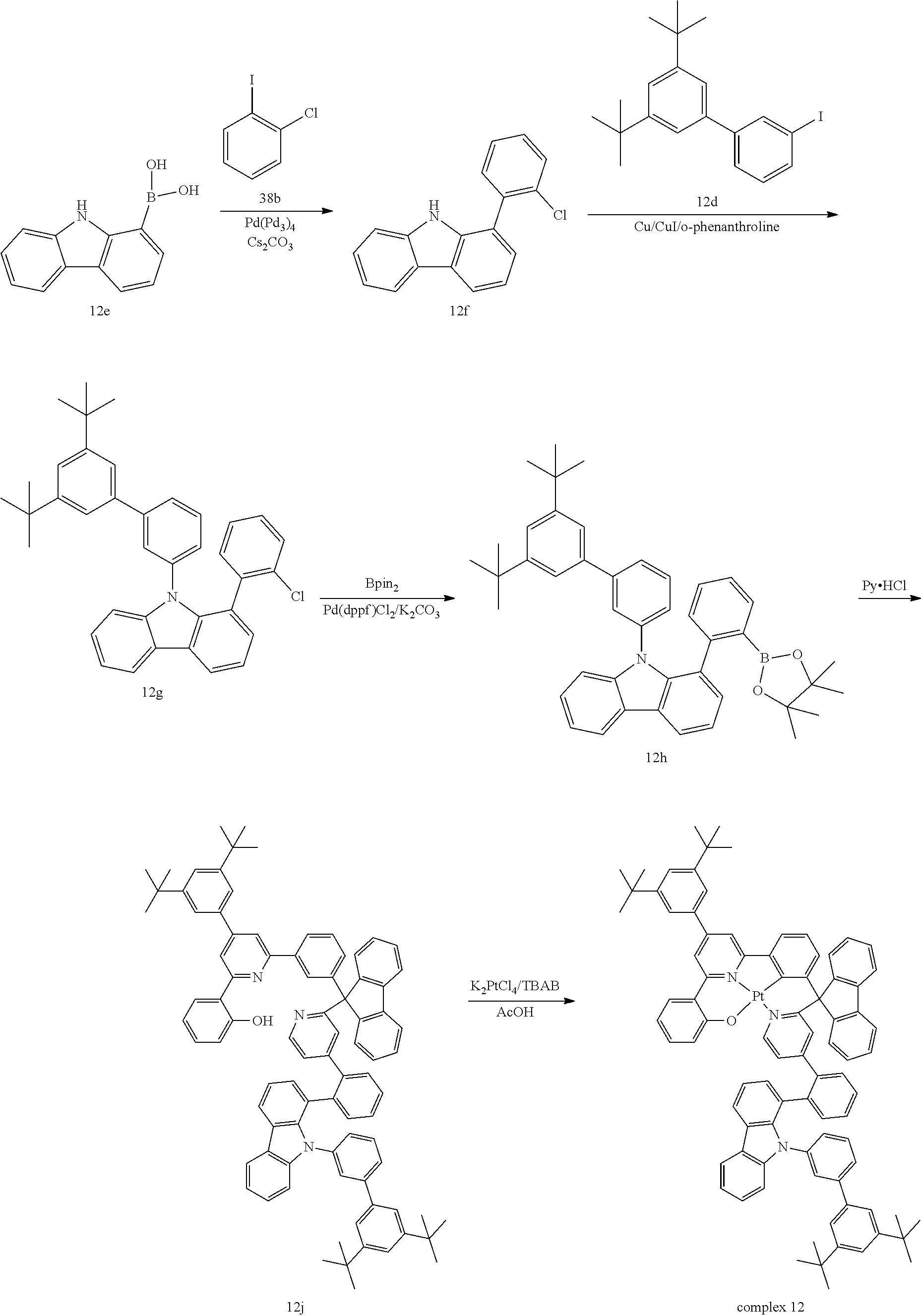

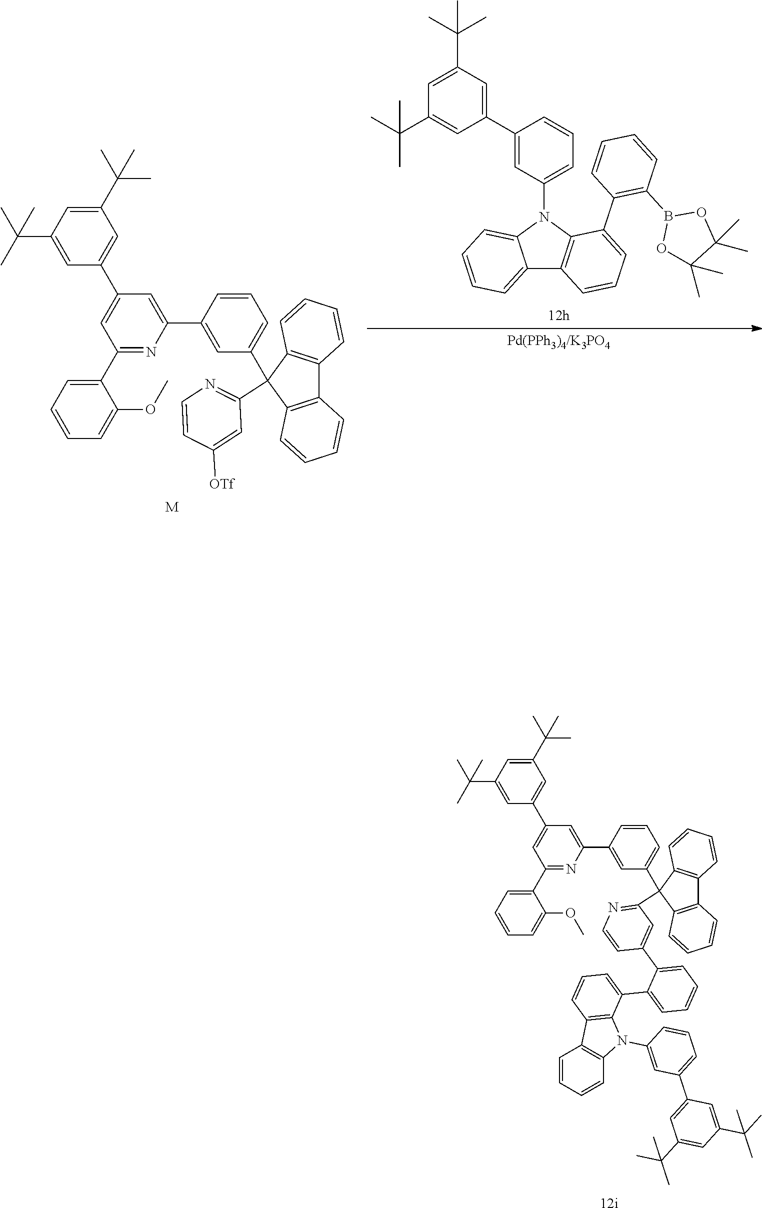

### Synthesis of Intermediate 12c

A 1,000 mL single-necked flask was charged with compound 12a (20 g, 74.29 mmol, 1.0 eq), compound 12b (12.66 g, 81.72 mmol, 1.1 eq), Pd(dppf)Cl₂ (2.72 g, 3.71 mmol, 0.05 eq) and K₂CO₃ (30.8 g, 222.87 mmol, 3.0 eq), dissolved in a solvent mixture of toluene/ethanol/water (400 mL/100 mL/100 mL). The system was stirred at 60° C. for 6 h under nitrogen atmosphere. After the reaction, most of the solvent was removed under reduced pressure. The residue was diluted with water and extracted with DCM (50 mL) three times. The combined organic layers were concentrated under reduced pressure and dry-loaded onto silica gel and purified by column chromatography (eluting agent:n-hexane:ethyl acetate=5:1, (V/V)) to afford 20 g of a yellow oil-like substance with a yield of 95.6%.

### Synthesis of Intermediate 12d

A 1,000 mL single-necked flask was charged with intermediate 12c (20 g, 71.06 mmol, 1.0 eq), CuI (17.59 g, 92.38 mmol, 1.3 eq), ^{t}BuONO (36.64 g, 36.64 g, 5.0 eq) and acetonitrile (400 mL), and reacted at 55° C. for 4 h under nitrogen atmosphere. After the reaction was completed, the reaction mixture was quickly filtered through a silica gel column (eluting agent: ethyl acetate), and the solvent was removed under reduced pressure. The crude product was purified by silica gel column chromatography (eluting agent: n-hexane) to afford 20 g of a white solid with a yield of 71.7%.

### Synthesis of Intermediate 12f

A 250 mL single-necked flask was charged with compound 12e (5 g, 23.7 mmol, 1.0 eq), compound 38b (6.77 g, 28.4 mmol, 1.2 eq), Pd(dppf)Cl₂ (866 mg, 1.18 mmol, 0.05 eq) and Cs₂CO₃ (15.4 g, 47.4 mmol, 2 eq), dissolved in toluene (100 mL) under nitrogen atmosphere for reaction at 100° C. for 16 h. After the reaction was completed, the reaction mixture was filtered, and the filtrate was concentrated under reduced pressure. The residue was purified by flash column chromatography on silica gel (eluting agent: n-hexane/ethyl acetate=10:1, (V/V)), to afford 5.28 g of a white solid, with a yield of 80%.

### Synthesis of Intermediate 12g

A 250 mL single-necked flask was charged with intermediate 12f (5.28 g, 19.0 mmol, 1.0 eq), 12d (11.17 g, 28.5 mmol, 1.5 eq), Cu (608 mg, 9.5 mmol, 0.5 eq), CuI (1.8 mg, 9.5 mmol, 0.5 eq), o-phenanthroline (3.42 g, 19.0 mmol, 1.0 eq), and Cs₂CO₃ (18.53 g, 57.0 mmol, 3.0 eq), dissolved in xylene (150 mL) under nitrogen atmosphere for reaction at 140° C. for 48 h. After the reaction was completed, the mixture was rapidly filtered through a silica gel column using ethyl acetate (EA) as the eluting agent. The solvent was removed under reduced pressure, and the crude product was purified by column chromatography on silica gel (eluting agent:hexane:ethyl acetate=10:1 (V/V)) to afford 7.6 g of a white solid with a yield of 74%.

### Synthesis of Intermediate 12h

A 250 mL single-mouth flask was charged with intermediate 12g (7.6 g, 14.0 mmol, 1.0 eq), Bpin₂ (5.33 g, 21.0 mmol, 1.5 eq), Pd(dppf)Cl₂ (511 mg, 0.7 mmol, 0.05 eq) and K₂CO₃ (3.86 g, 28.0 mmol, 2 eq), dissolved in toluene (100 mL) under nitrogen atmosphere for reaction at 100° C. for 16 h. After the reaction was completed, the reaction mixture was filtered, and the filtrate was concentrated under reduced pressure. The residue was purified by flash column chromatography on silica gel (eluting agent: n-hexane/ethyl acetate=10:1, (V/V)) to afford 7.1 g of a white solid with a yield of 80%.

### Synthesis of Intermediate 12i

A 250 mL single-necked flask was charged with intermediate M (5 g, 5.96 mmol, 1.0 eq), 12h (3.95 g, 7.15 mmol, 1.2 eq), Pd(PPh₃)₄ (304 mg, 0.26 mmol, 0.05 eq) and K₃PO₄ (2.23 g, 10.52 mmol, 2 eq), dissolved in a solvent mixture of Tol/EtOH/H₂O (30 mL/30 mL/15 mL) under nitrogen atmosphere for reaction at 80° C. for 16 h. After the reaction was completed, the mixture was filtered, and the filtrate was concentrated under reduced pressure. The residue was purified by flash column chromatography on silica gel (eluting agent: n-hexane/ethyl acetate=10:1, (V/V)) to afford 5.68 g of a white solid with a yield of 80%.

### Synthesis of Intermediate 12j

A 100 mL single-necked flask was charged with intermediate 12i (5.68 g), pyridine hydrochloride (56.8 g) and o-dichlorobenzene (6 mL). Under nitrogen protection, the mixture was heated at 200° C. in oil bath for reaction for 8 h, and then cooled to room temperature. The crude product was dissolved in copious amounts of water and extracted with DCM for three times. The organic layers were concentrated under reduced pressure, dry-loaded onto silica gel and purified by column chromatography (elution solvent:n-hexane:ethyl acetate=5:1, (V/V)) to afford 5.2 g of a pale yellow solid with a yield of 93%.

### Synthesis of Compound 12

A 250 mL single-necked flask was charged with intermediate 12j (200 mg, 0.17 mmol), K₂PtCl₄ (76 mg, 0.20 mmol), TBAB (5 mg, 0.017 mmol) and acetic acid (5 mL) under nitrogen atmosphere, and reacted at 130° C. for 48 h. After the reaction was completed, excess deionized water was added to precipitate the solid, which was filtered and dissolved in dichloromethane, and then concentrated under reduced pressure. The residue was dry-loaded onto silica gel and purified by column chromatography (elution solvent: Hex:DCM:EA=20:20:1, (V/V/V)). The collected product was recrystallized from DCM/n-hexane=1:4 (V/V) to afford 150 mg of a red solid.

¹H NMR (500 MHz, Chloroform-d) δ 8.47 (d, J=4.9 Hz, 1H), 8.20 (s, 1H), 8.13 (s, 1H), 7.99 (dd, J=7.5, 2.0 Hz, 1H), 7.88 (s, 2H), 7.85 (s, 1H), 7.82 (t, J=1.9 Hz, 1H), 7.75-7.70 (m, 2H), 7.69-7.66 (m, 2H), 7.66-7.62 (m, 3H), 7.62-7.59 (m, 2H), 7.56-7.49 (m, 2H), 7.49-7.37 (m, 15H), 7.35 (s, 1H), 7.33-7.28 (m, 2H), 7.26 (s, 1H), 7.23 (td, J=7.5, 2.0 Hz, 1H), 7.19 (dd, J=7.4, 2.0 Hz, 1H), 1.36 (s, 36H).

### Example 5

An organic light emitting diode was prepared using the compound of the present disclosure as the light-emitting material, with the device structure shown in the FIGURE.

First, a glass substrate (represented by 10 in the FIGURE) with a transparent conductive positive electrode ITO (represented by 20 in the FIGURE) was sequentially washed with detergent solution and deionized water, ethanol, acetone, and deionized water, and then treated with oxygen plasma for 30 seconds.

Then, HATCN was vacuum-deposited on the ITO to a thickness of 10 nm as a hole injection layer (represented by 30 in the FIGURE).

Then, compound HT was vacuum-deposited to form a 40 nm thick hole transport layer (represented by 40 in the FIGURE).

Then, a 20 nm thick light-emitting layer (represented by 50 in the FIGURE) was vacuum-deposited on the hole transport layer, consisting of a mixed doping of platinum compound 114 (20%) and CBP (80%).

Then, AlQ₃ was vacuum-deposited on the light-emitting layer to a thickness of 40 nm as an electron transport layer (represented by 60 in the FIGURE).

Finally, 1 nm LiF was vacuum-deposited as the electron injection layer (represented by 70 in the FIGURE) and 100 nm Al was used as the device cathode (represented by 80 in the FIGURE).

### Example 6

An organic light emitting diode was prepared by the method described in Example 5 using Compound 9 instead of Compound 114.

### Example 7

An organic light emitting diode was prepared by the method described in Example 5 using Compound 5 instead of Compound 114.

### Example 8

An organic light emitting diode was prepared by the method described in Example 5 using Compound 12 instead of Compound 114.

### Comparative Example 1

The organic light emitting diode was prepared by the method described in Example 5 using compound Ref-1 (CN110872325A) instead of compound 114.

### Comparative Example 2

The organic light emitting diode was prepared by the method described in Example 5 using compound Ref-3 (CN110872325A) instead of compound 114.

### Comparative Example 3

The organic light emitting diode was prepared by the method described in Example 5 using compound Ref-4 (CN110872325A) instead of compound 114.

The structural formulas of HATCN, HT, AlQ₃, Ref-1, Ref-2, Ref-3 and CBP in the device are as follows:

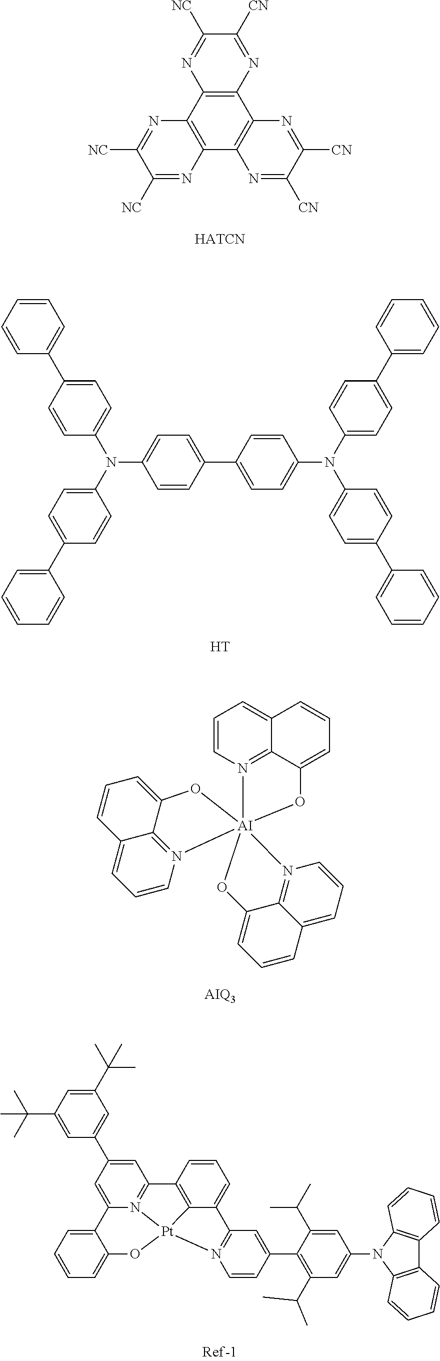

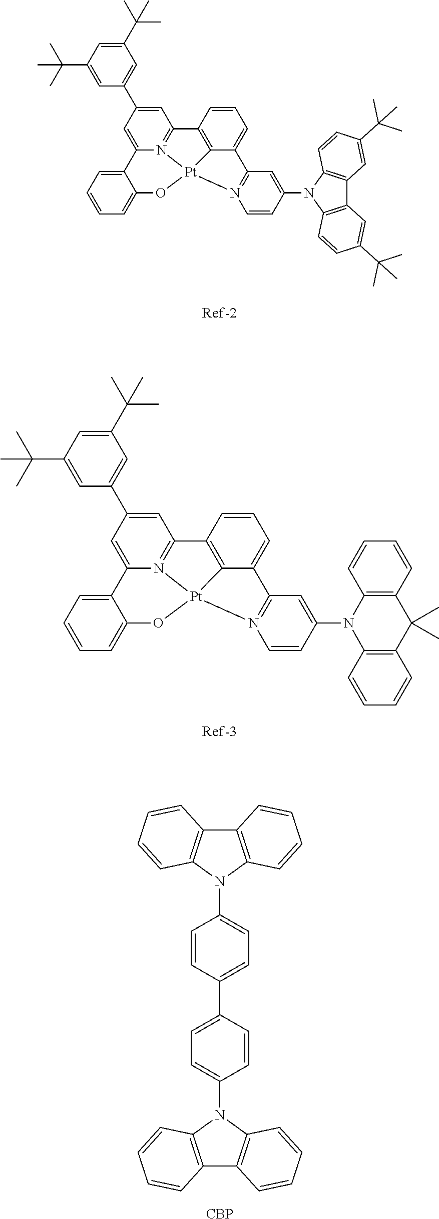

The device performance of the organic electroluminescent devices in Examples 5-8 and Comparative Examples 1-3 at a current density of 20 mA/cm² is listed in the following table:

| | | Driving | Luminous | Device |
|---|---|---|---|---|
| Device Number | Compound | Voltage | Efficiency | Lifetime (LT95) |
| Example 5 | Compound 114 | 1 | 1 | 1 |
| Example 6 | Compound 9 | 0.9 | 1 | 0.96 |
| Example 7 | Compound 5 | 1 | 1.1 | 1.1 |
| Example 8 | Compound 12 | 1 | 1.3 | 1.2 |
| Comparative | Ref-1 | 1.08 | 0.87 | 0.31 |
| Example 1 | | | | |
| Comparative | Ref-2 | 1.06 | 0.67 | 0.33 |
| Example 2 | | | | |
| Comparative | Ref-3 | 1.04 | 0.71 | 0.06 |
| Example 3 | | | | |

Note:
the device performance test was based on Example 5, with each index set to 1; LT95 represents the time when the device brightness decays to 95% of the initial brightness (10,000 cd/m²).

It can be seen from the data in the table that under the same conditions, the platinum compound material of the present disclosure is applied to an organic light emitting diode, which has a lower driving voltage and a higher luminous efficiency. In addition, the device lifetime of the organic light-emitting diode based on the compound of the present disclosure is significantly better than that of the compound material in the comparative examples, which can meet the requirements of the display industry for light-emitting materials and has excellent industrialization prospects.

The above-mentioned various embodiments are only for examples and are not intended to limit the scope of the present disclosure. Without departing from the spirit of the present disclosure, various materials and structures in the present disclosure may be replaced with other materials and structures. It should be understood that those skilled in the art can make many modifications and changes based on the concept of the present disclosure without creative work. Therefore, any technical solutions that can be obtained by technicians through analysis, reasoning or partial research on the basis of existing technologies should be within the scope of protection limited by the claims.

1. A spiro-containing platinum compound, having a structure of formula (I): 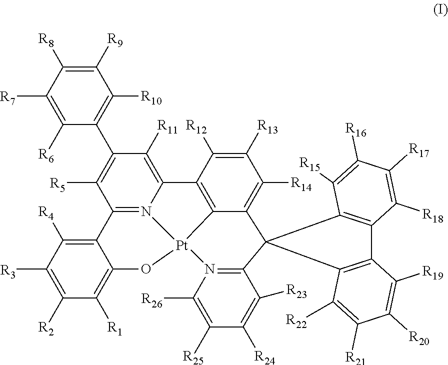
wherein R₁-R₂₆ are each independently selected from a group consisting of hydrogen, deuterium, halogen, a substituted or unsubstituted alkyl having 1 to 20 carbon atoms, a substituted or unsubstituted cycloalkyl having 3 to 20 ring carbon atoms, a substituted or unsubstituted heteroalkyl having 1 to 20 carbon atoms, a substituted or unsubstituted aralkyl having 7 to 30 carbon atoms, a substituted or unsubstituted alkoxy having 1 to 20 carbon atoms, a substituted or unsubstituted aryloxy having 6 to 30 carbon atoms, a substituted or unsubstituted alkenyl having 2 to 20 carbon atoms, a substituted or unsubstituted aryl having 6 to 40 carbon atoms, a substituted or unsubstituted heteroaryl having 3 to 40 carbon atoms, a substituted or unsubstituted alkylsilyl having 3 to 20 carbon atoms, a substituted or unsubstituted arylsilyl having 6 to 20 carbon atoms, a substituted or unsubstituted amino group having 0 to 20 carbon atoms, a substituted or unsubstituted acyl having 1 to 20 carbon atoms, carboxy, a substituted or unsubstituted ester group having 1 to 20 carbon atoms, cyano, isocyano, a substituted or unsubstituted sulfanyl having 0 to 20 carbon atoms, a substituted or unsubstituted sulfinyl having 0 to 20 carbon atoms, a substituted or unsubstituted sulfonyl having 0 to 20 carbon atoms, or a substituted or unsubstituted phosphino having 0 to 20 carbon atoms;
wherein groups with adjacent serial numbers among R₆-R₁₀ are capable of being bonded to form a ring, groups with adjacent serial numbers among R₁₅-R₁₈ are capable of being bonded to form a ring, and groups with adjacent serial numbers among R₁₉-R₂₂ are capable of being bonded to form a ring;
wherein substitution refers to being substituted with deuterium, halogen, amino, nitro, cyano, an aryl having 6 to 20 carbon atoms, a heteroaryl having 5 to 10 carbon atoms, or C1-C4 alkyl; and
wherein the heteroaryl and the heteroalkyl each contain at least one heteroatom selected from S, N or O.

2. The spiro-containing platinum compound according to claim 1, wherein at least one of R₂₃-R₂₆ is selected from a group consisting of a substituted or unsubstituted alkyl having 1 to 20 carbon atoms, a substituted or unsubstituted cycloalkyl having 3 to 20 ring carbon atoms, a substituted or unsubstituted heteroalkyl having 1 to 20 carbon atoms, a substituted or unsubstituted aralkyl having 7 to 30 carbon atoms, a substituted or unsubstituted alkoxy having 1 to 20 carbon atoms, a substituted or unsubstituted aryloxy having 6 to 30 carbon atoms, a substituted or unsubstituted alkenyl having 2 to 20 carbon atoms, a substituted or unsubstituted aryl having 6 to 40 carbon atoms, a substituted or unsubstituted heteroaryl having 3 to 40 carbon atoms, a substituted or unsubstituted alkylsilyl having 3 to 20 carbon atoms, or a substituted or unsubstituted arylsilyl having 6 to 20 carbon atoms.

3. The spiro-containing platinum compound according to claim 2, wherein R₁-R₂₂ are each independently selected from a group consisting of hydrogen, deuterium, halogen, amino, carboxyl, a substituted or unsubstituted sulfanyl having 1 to 20 carbon atoms, cyano, a substituted or unsubstituted sulfonyl having 0 to 20 carbon atoms, a substituted or unsubstituted phosphino having 0 to 20 carbon atoms, a substituted or unsubstituted alkyl having 1 to 20 carbon atoms, a substituted or unsubstituted cycloalkyl having 3 to 20 ring carbon atoms, a substituted or unsubstituted aryl having 6 to 10 carbon atoms, a substituted or unsubstituted heteroaryl having 3 to 10 carbon atoms, a substituted or unsubstituted alkylsilyl having 3 to 10 carbon atoms, or a substituted or unsubstituted arylsilyl having 6 to 10 carbon atoms;
one of R₂₃-R₂₆ is selected from a group consisting of a substituted or unsubstituted alkyl having 1 to 20 carbon atoms, a substituted or unsubstituted cycloalkyl having 3 to 20 ring carbon atoms, a substituted or unsubstituted heteroalkyl having 1 to 20 carbon atoms, a substituted or unsubstituted aralkyl having 7 to 30 carbon atoms, a substituted or unsubstituted alkoxy having 1 to 20 carbon atoms, a substituted or unsubstituted aryloxy having 6 to 30 carbon atoms, a substituted or unsubstituted alkenyl having 2 to 20 carbon atoms, a substituted or unsubstituted aryl having 6 to 30 carbon atoms, a substituted or unsubstituted heteroaryl having 3 to 30 carbon atoms, a substituted or unsubstituted alkylsilyl having 3 to 20 carbon atoms, or a substituted or unsubstituted arylsilyl having 6 to 20 carbon atoms; and the remaining three of R₂₃-R₂₆ are hydrogen.

4. A spiro-containing platinum compound, having a structure of formula (II): 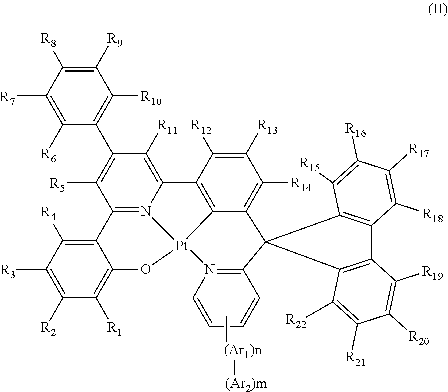
wherein Ar₁ and Ar₂ are each independently selected from a substituted or unsubstituted aryl having 6 to 40 carbon atoms, or a substituted or unsubstituted heteroaryl having 3 to 40 carbon atoms; n and m are integers from 0 to 2, and m+n≥1;
wherein R₁-R₂₂ are each independently selected from a group consisting of hydrogen, deuterium, halogen, a substituted or unsubstituted alkyl having 1 to 20 carbon atoms, a substituted or unsubstituted cycloalkyl having 3 to 20 ring carbon atoms, a substituted or unsubstituted heteroalkyl having 1 to 20 carbon atoms, a substituted or unsubstituted aralkyl having 7 to 30 carbon atoms, a substituted or unsubstituted alkoxy having 1 to 20 carbon atoms, a substituted or unsubstituted aryloxy having 6 to 30 carbon atoms, a substituted or unsubstituted alkenyl having 2 to 20 carbon atoms, a substituted or unsubstituted aryl having 6 to 40 carbon atoms, a substituted or unsubstituted heteroaryl having 3 to 40 carbon atoms, a substituted or unsubstituted alkylsilyl having 3 to 20 carbon atoms, a substituted or unsubstituted arylsilyl having 6 to 20 carbon atoms, a substituted or unsubstituted amino group having 0 to 20 carbon atoms, a substituted or unsubstituted acyl having 1 to 20 carbon atoms, carboxy, a substituted or unsubstituted ester group having 1 to 20 carbon atoms, cyano, isocyano, a substituted or unsubstituted sulfanyl having 0 to 20 carbon atoms, a substituted or unsubstituted sulfinyl having 0 to 20 carbon atoms, a substituted or unsubstituted sulfonyl having 0 to 20 carbon atoms, or a substituted or unsubstituted phosphino having 0 to 20 carbon atoms;
wherein groups with adjacent serial numbers among R₆-R₁₀ are capable of being bonded to form a ring, groups with adjacent serial numbers among R₁₅-R₁₈ are capable of being bonded to form a ring, and groups with adjacent serial numbers among R₁₉-R₂₂ are capable of being bonded to form a ring;
wherein substitution refers to being substituted with deuterium, halogen, amino, nitro, cyano, an aryl having 6 to 20 carbon atoms, a heteroaryl having 5 to 10 carbon atoms, or C1-C4 alkyl; and
wherein the heteroaryl and the heteroalkyl each contain at least one heteroatom selected from S, N or O.

5. The spiro-containing platinum compound according to claim 4, wherein Ar₁ is independently selected from a substituted or unsubstituted conjugated fused-ring aryl having 6 to 20 carbon atoms, a substituted or unsubstituted conjugated fused-ring heteroaryl having 6 to 20 carbon atoms, or a substituted or unsubstituted non-fused-ring aryl having 6 to 20 carbon atoms; and Ar₂ is independently selected from a substituted or unsubstituted aryl having 6 to 20 carbon atoms, or a substituted or unsubstituted heteroaryl having 3 to 20 carbon atoms.

6. The spiro-containing platinum compound according to claim 5, wherein R₁-R₂₂ are each selected from a group consisting of hydrogen, deuterium, halogen, amino, cyano, a substituted or unsubstituted alkyl having 1 to 10 carbon atoms, a substituted or unsubstituted cycloalkyl having 3 to 10 ring carbon atoms, a substituted or unsubstituted aryl having 6 to 10 carbon atoms, a substituted or unsubstituted heteroaryl having 3 to 10 carbon atoms, a substituted or unsubstituted alkylsilyl having 3 to 10 carbon atoms, or a substituted or unsubstituted arylsilyl having 6 to 10 carbon atoms.

7. The spiro-containing platinum compound according to claim 6, wherein among R₁-R₂₂, R₇, R₉, R₁₃ and R₁₅-R₂₂ are each independently selected from a group consisting of hydrogen, deuterium, halogen, amino, cyano, or an alkyl group having 1 to 6 carbon atoms; and R₁-R₆, R₈, R₁₀-R₁₂ and R₁₄ are hydrogen.

8. The spiro-containing platinum compound according to claim 7, wherein Ar₁ is selected from a group consisting of a substituted or unsubstituted phenyl, a substituted or unsubstituted biphenyl, a substituted or unsubstituted naphthyl, a substituted or unsubstituted anthracyl, or a substituted or unsubstituted carbazolyl; Ar₂ is selected from a group consisting of a substituted or unsubstituted phenyl, a substituted or unsubstituted carbazolyl, a substituted or unsubstituted fluorenyl, a substituted or unsubstituted indolyl, a substituted or unsubstituted dibenzofuranyl, a substituted or unsubstituted dibenzothiophenyl, a substituted or unsubstituted acridinyl, a substituted or unsubstituted aza-anthracyl or oxo-anthracyl, or a substituted or unsubstituted phenanthrenyl; n and m are integers from 0 to 1.

9. The spiro-containing platinum compound according to claim 8, wherein among R₁-R₂₂, R₇, R₉, R₁₃, R₁₆, and R₂₁ are each independently selected from hydrogen, deuterium, or an alkyl group having 1 to 6 carbon atoms; and R₁-R₆, R₈, R₁₀-R₁₂, R₁₄-R₁₅, R₁₇-R₂₀ and R₂₂ are hydrogen;
wherein Ar₁ is selected from a substituted or unsubstituted phenyl or a substituted or unsubstituted carbazolyl; Ar₂ is selected from a substituted or unsubstituted phenyl or a substituted or unsubstituted carbazolyl; and
wherein substitution refers to being substituted with deuterium, halogen, amino, nitro, cyano, an aryl having 6 to 20 carbon atoms, a heteroaryl having 5 to 10 carbon atoms, or C1-C4 alkyl.

10. A spiro-containing platinum compound, having one of the following structures:

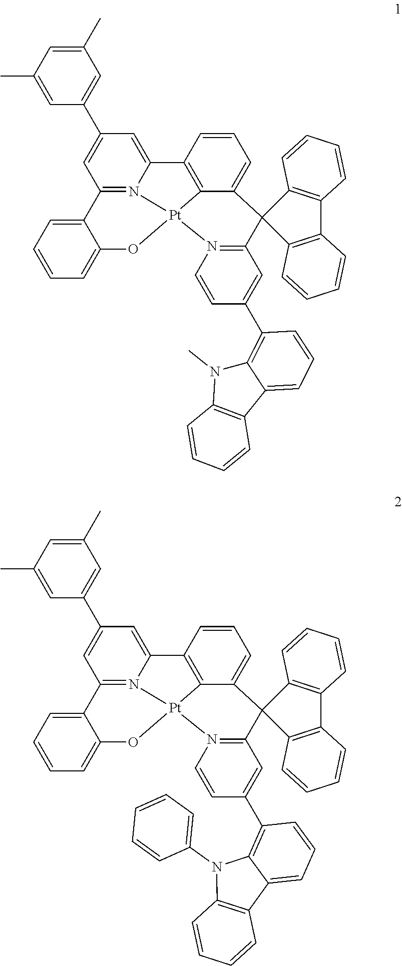

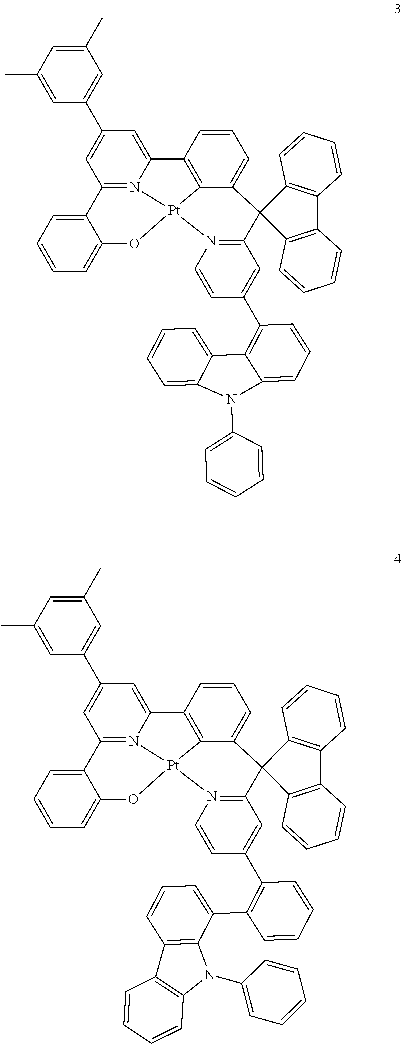

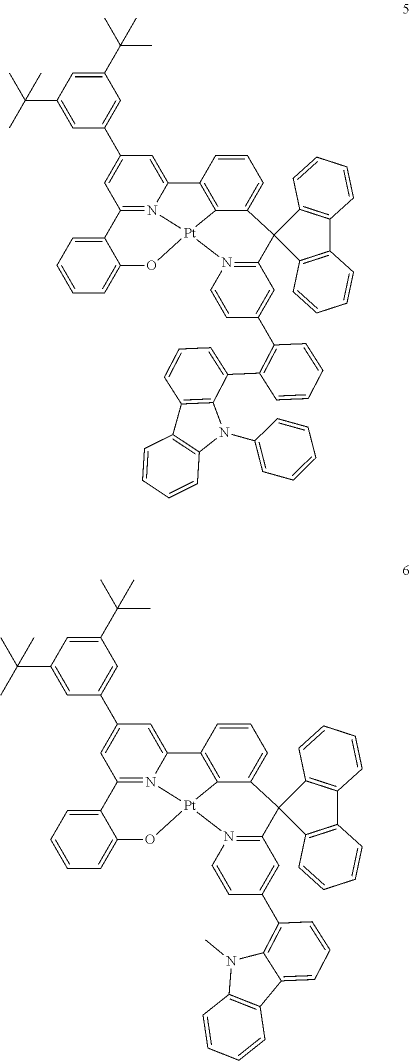

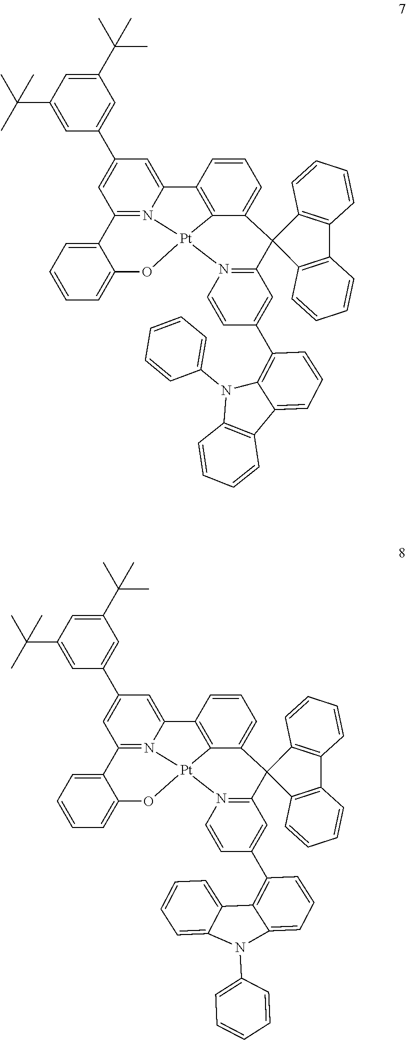

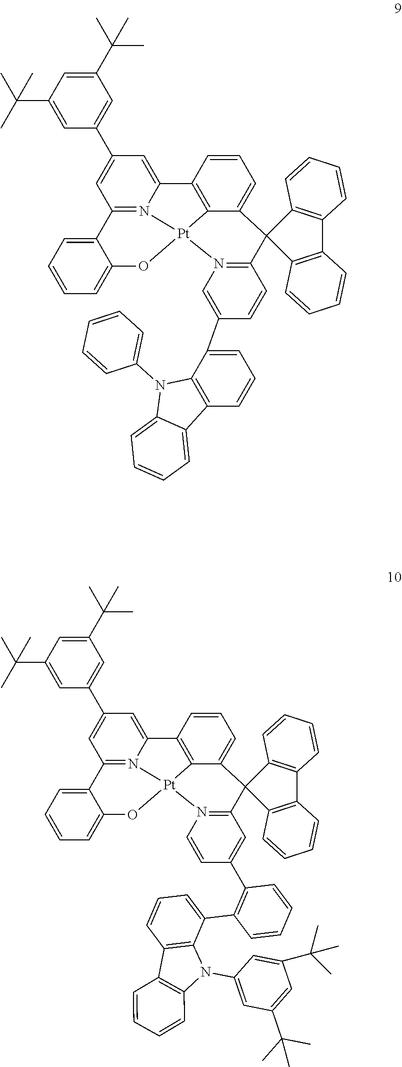

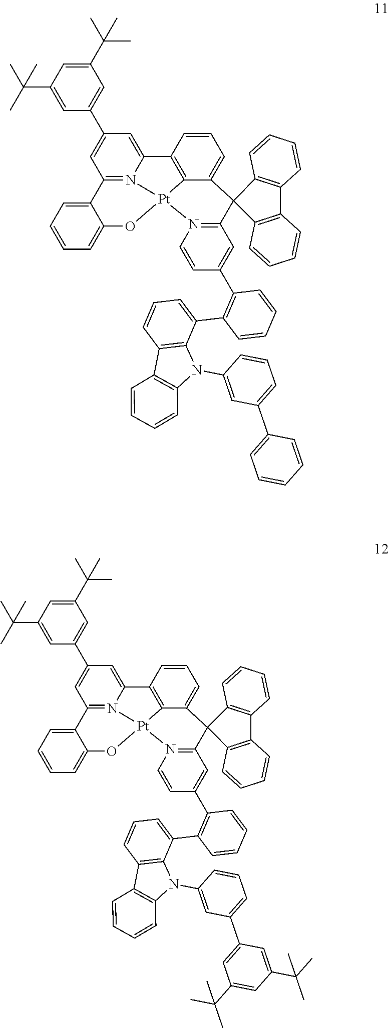

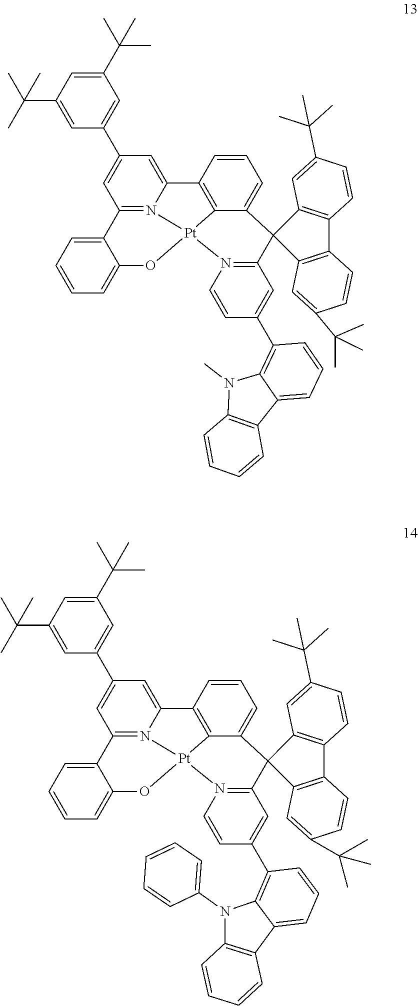

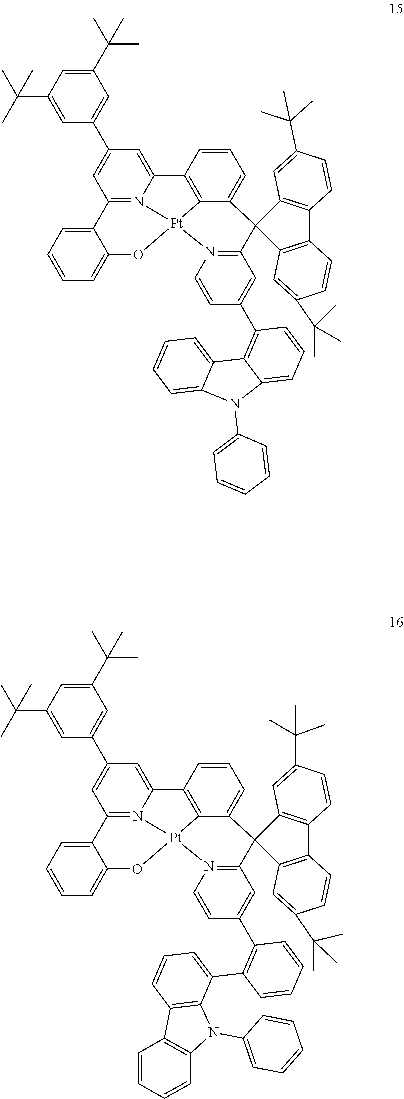

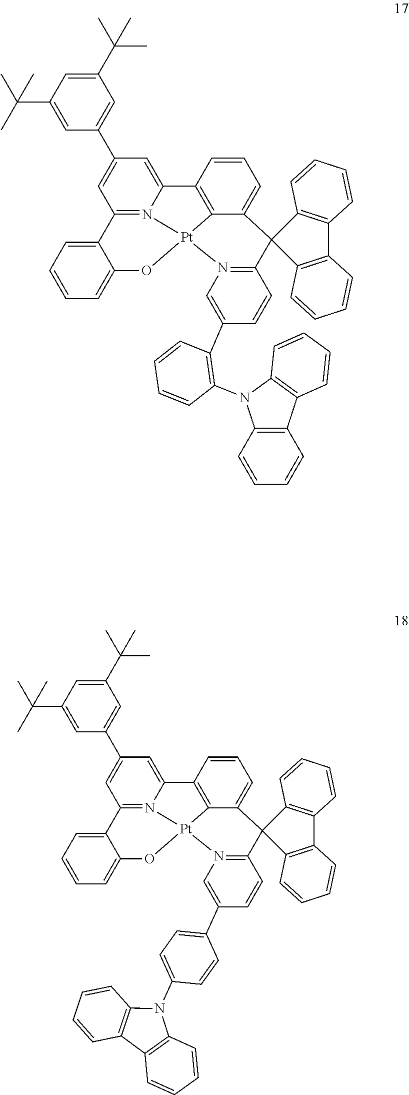

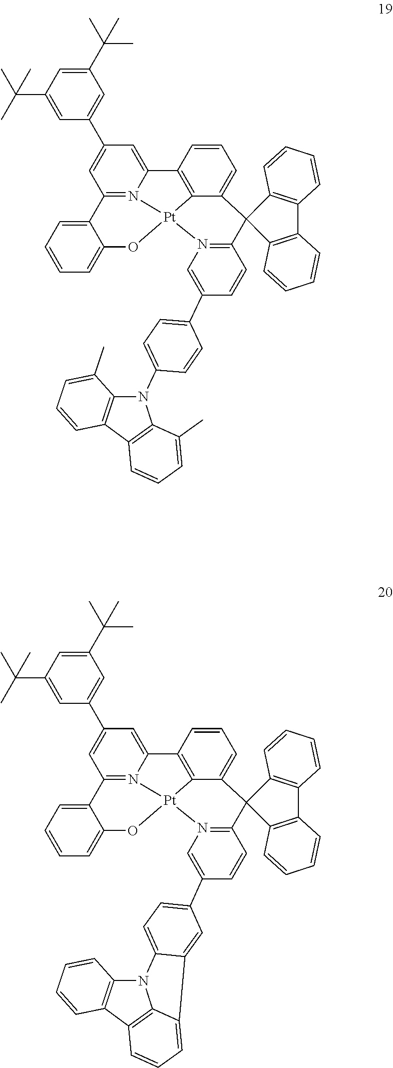

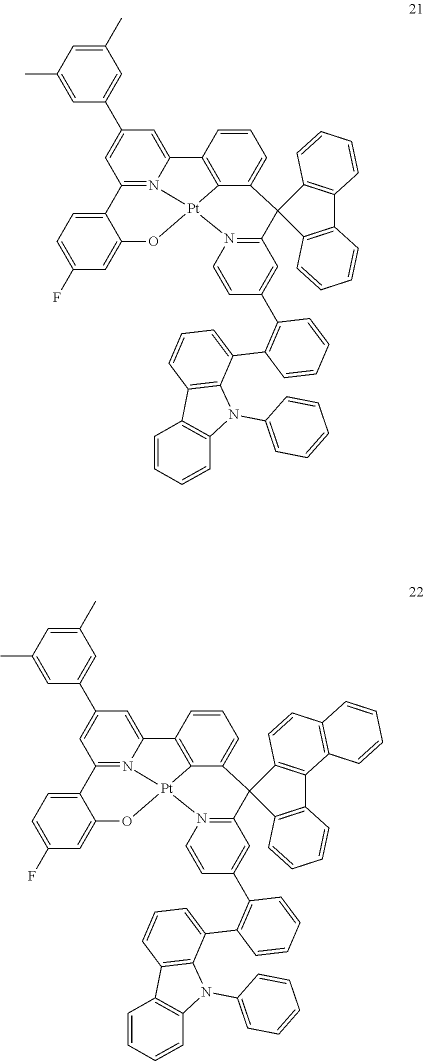

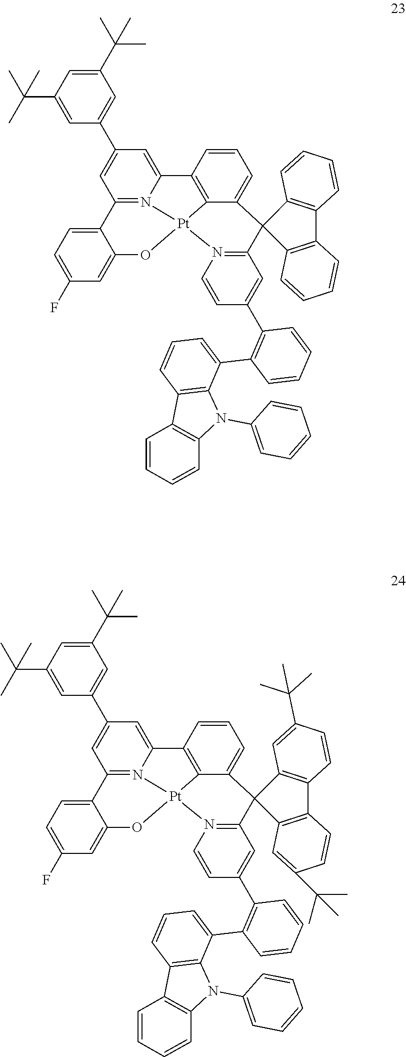

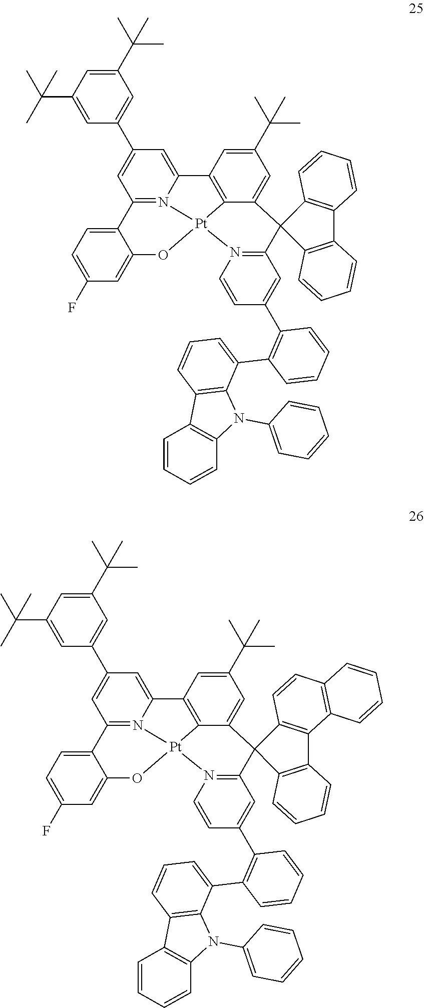

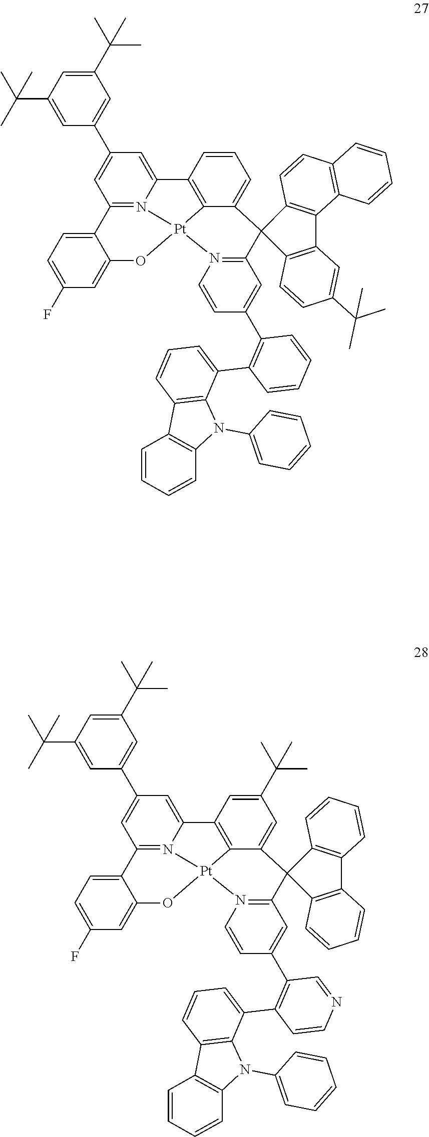

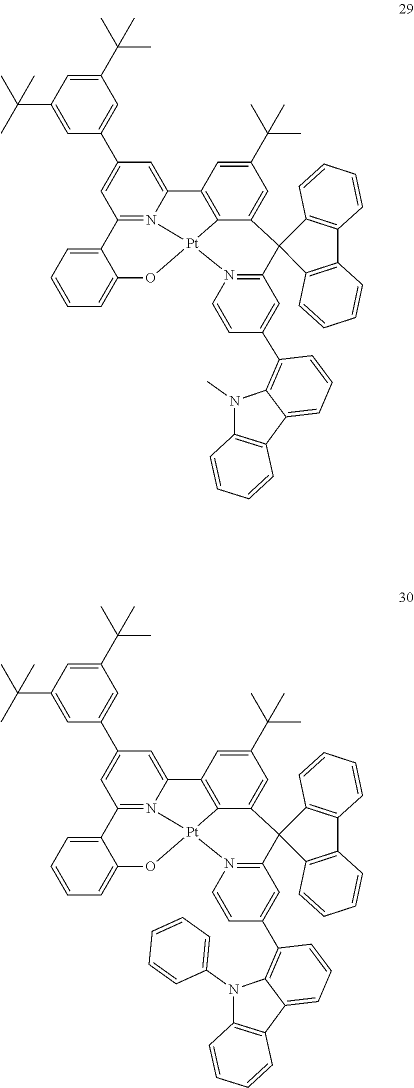

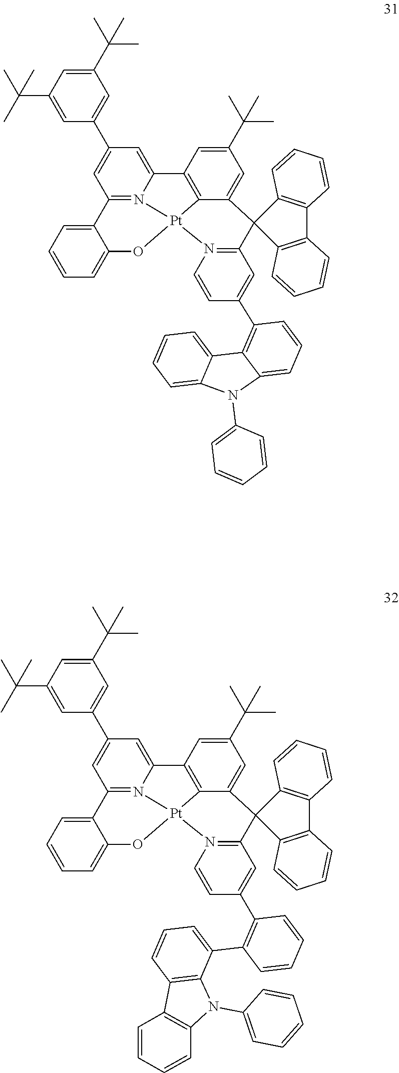

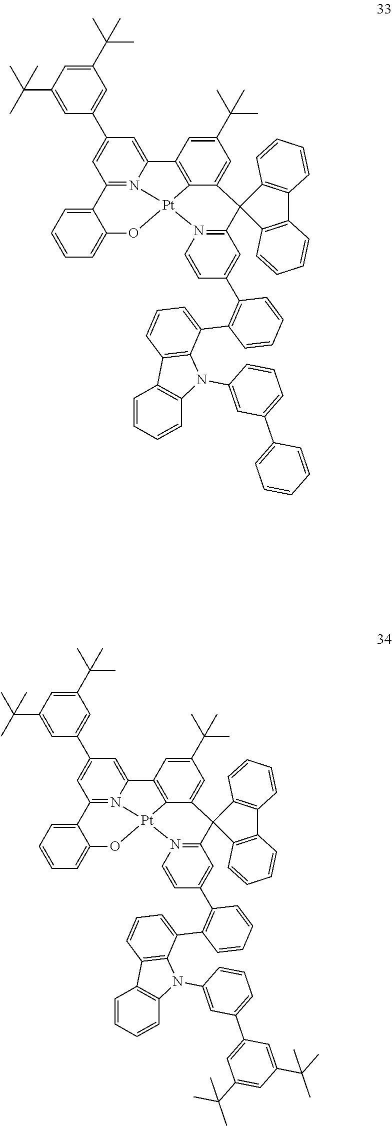

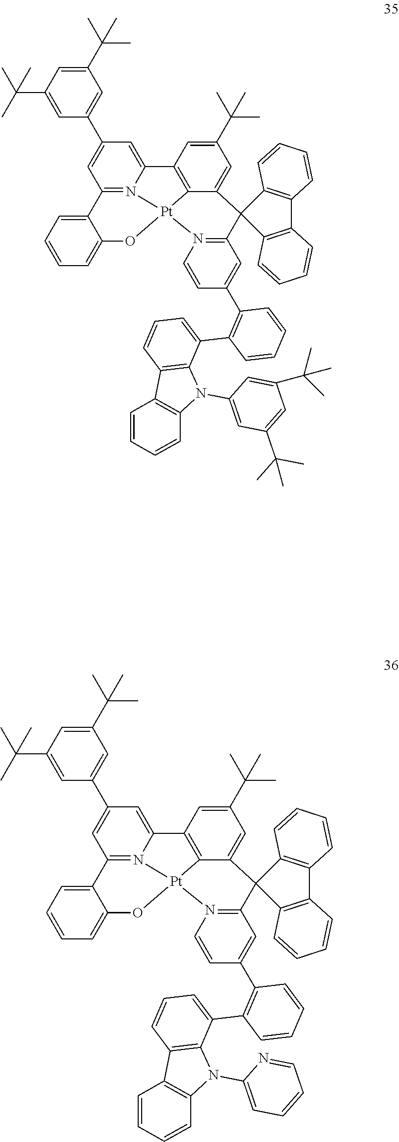

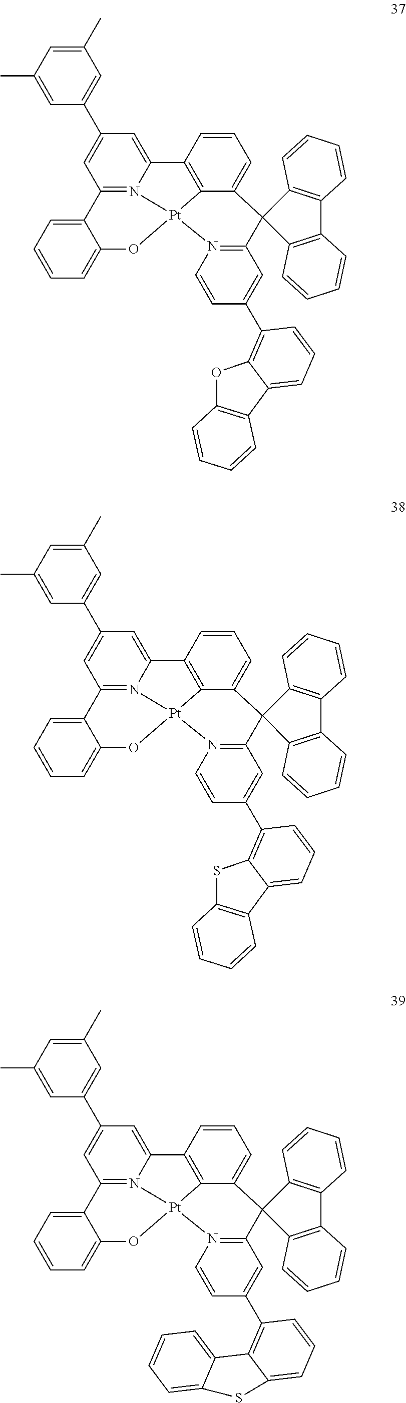

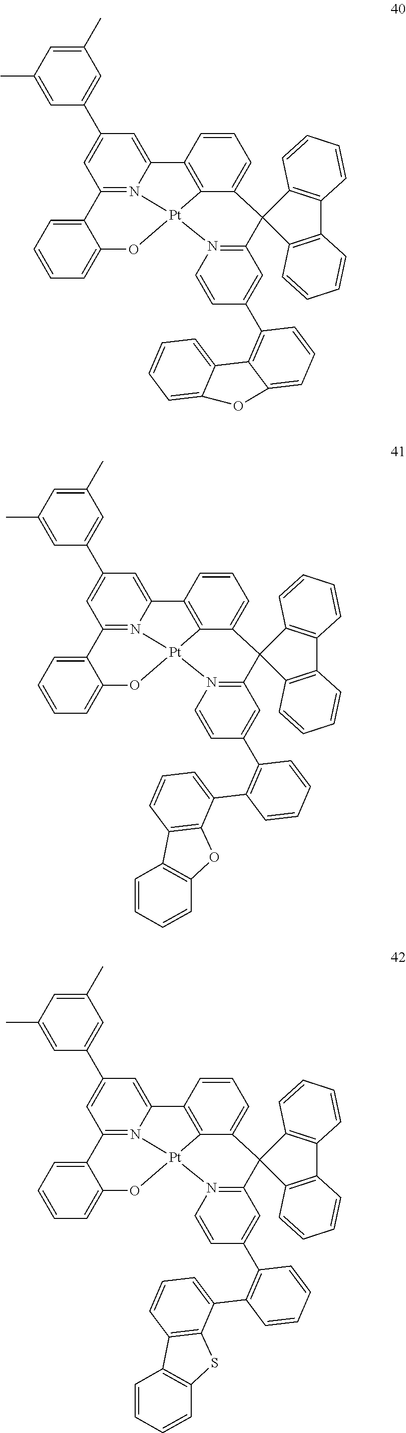

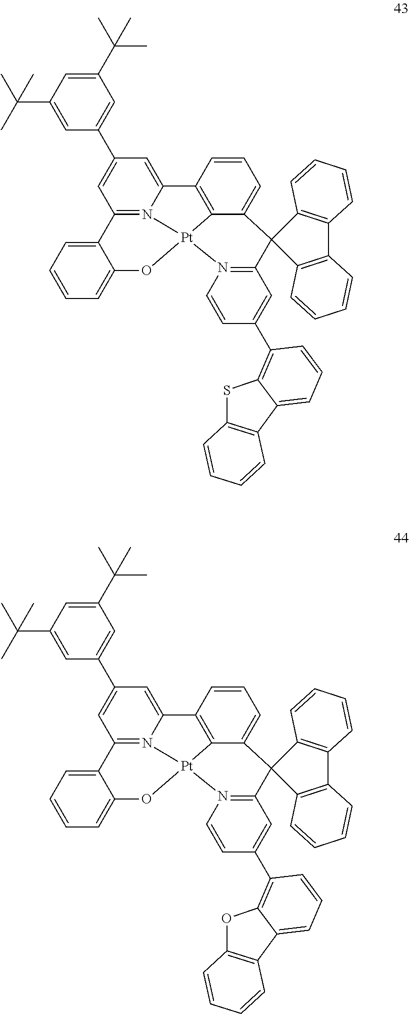

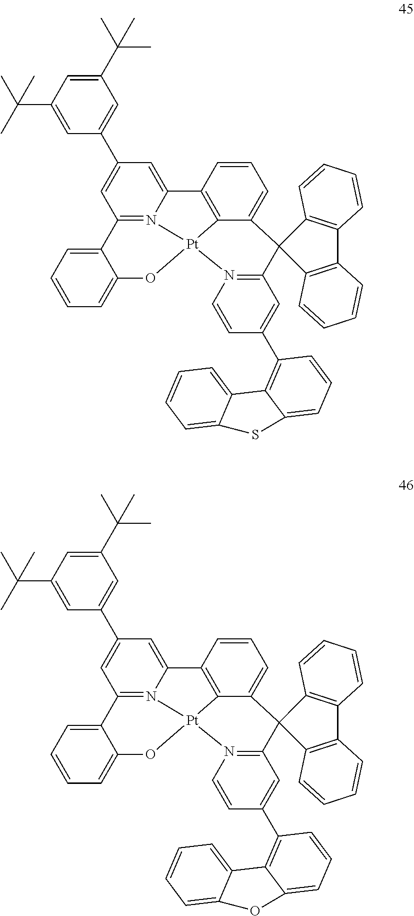

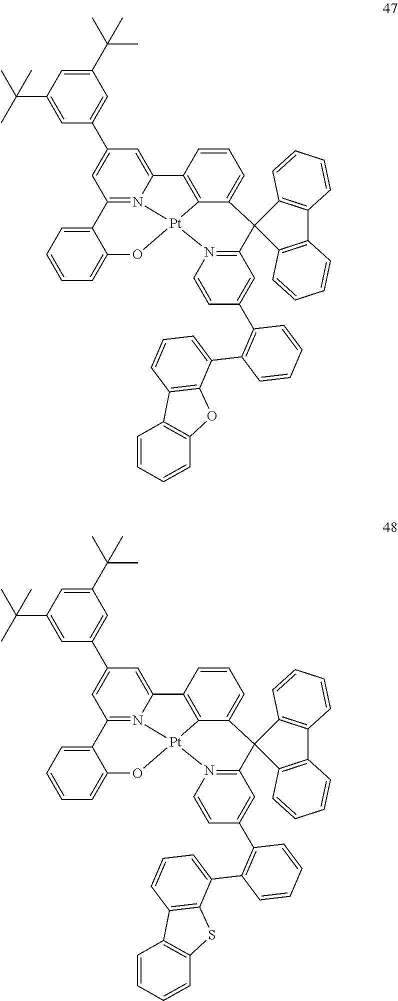

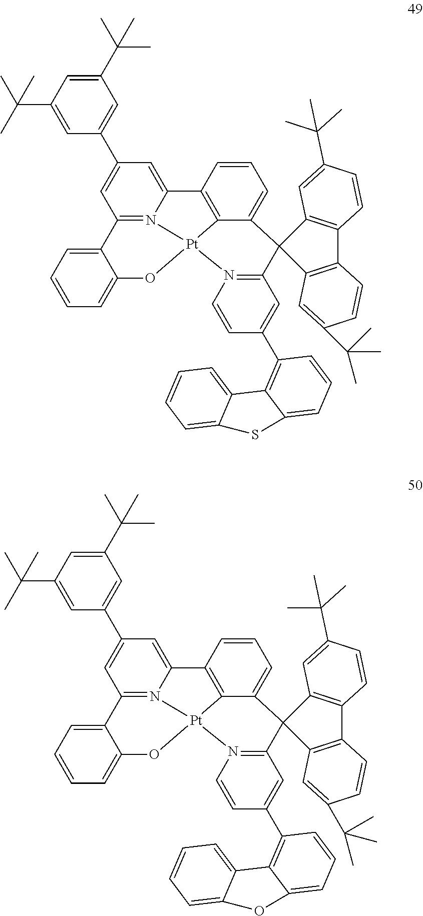

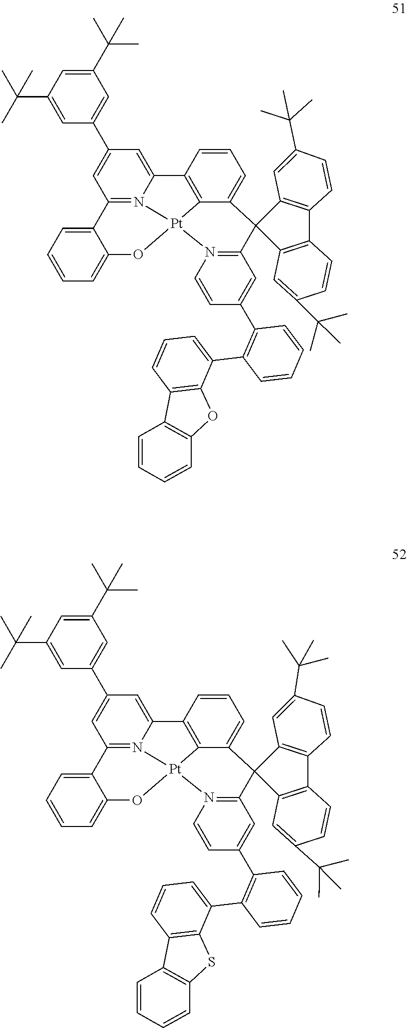

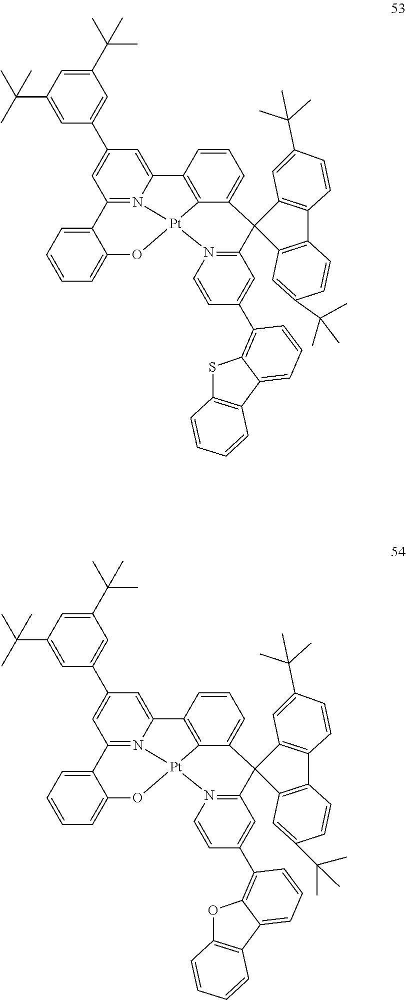

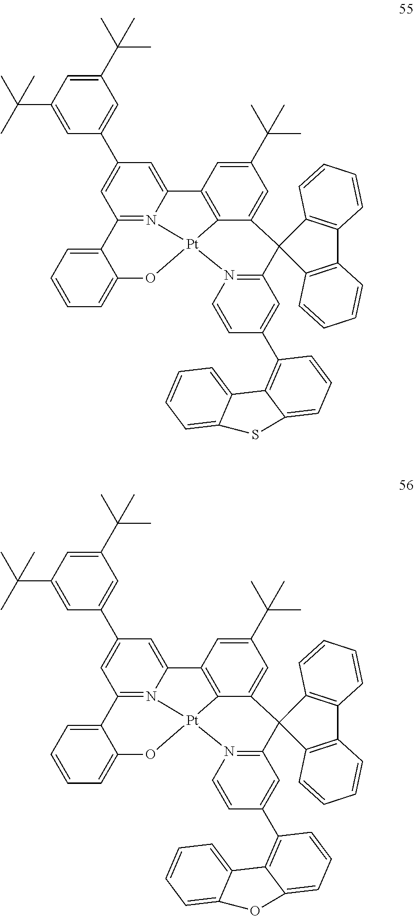

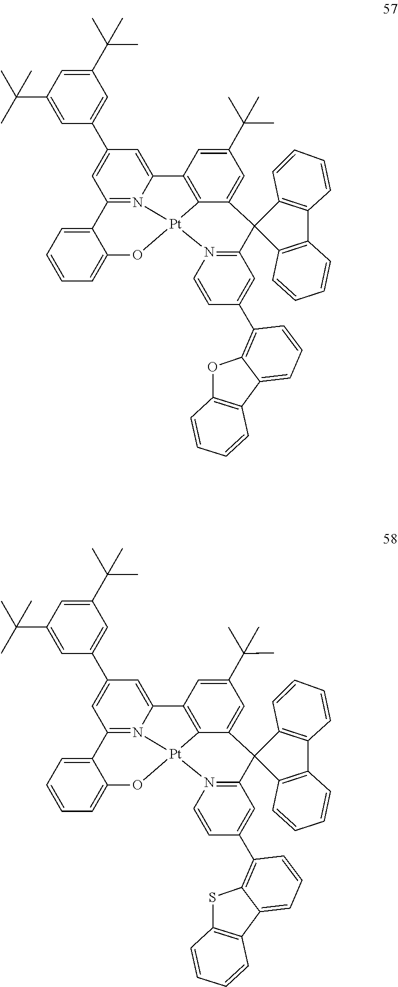

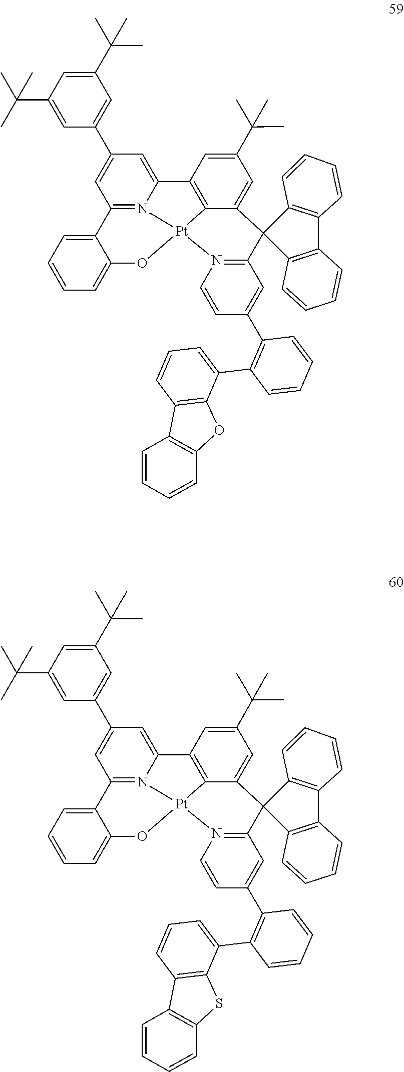

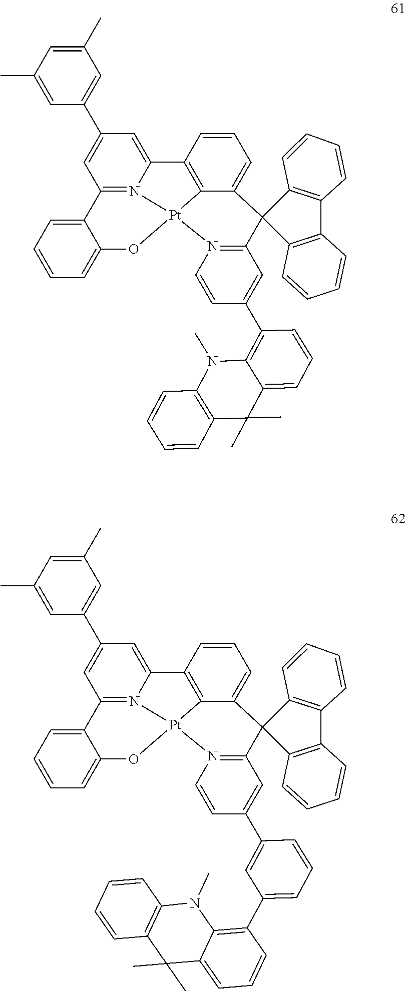

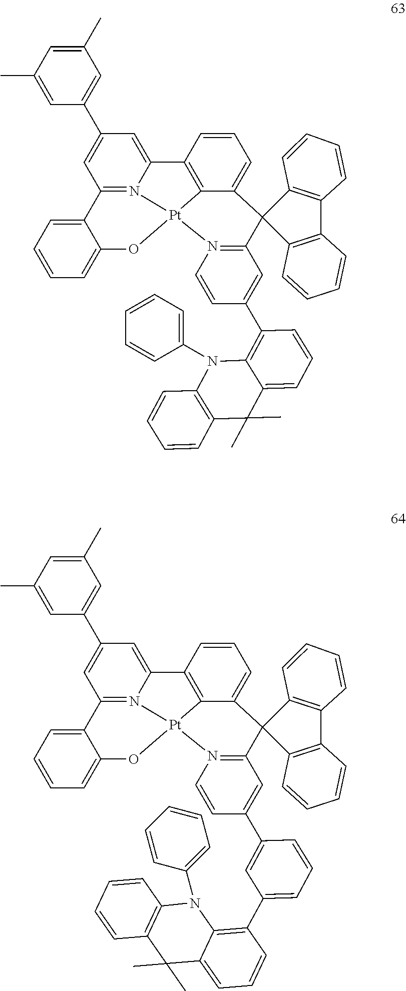

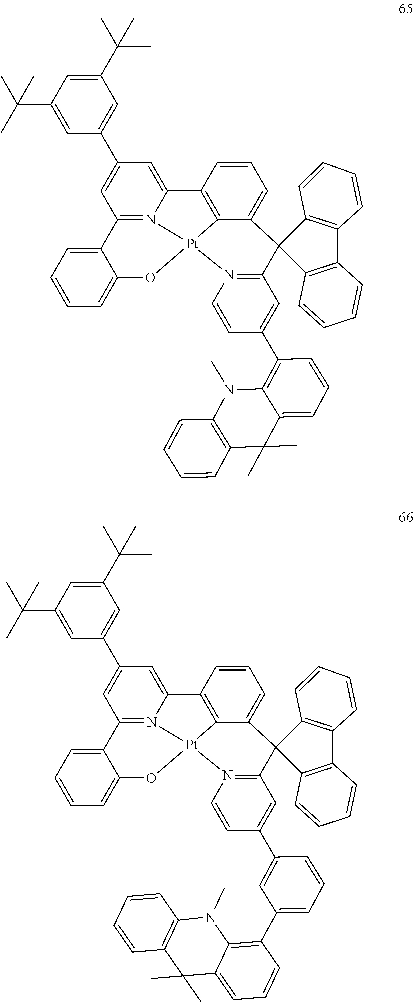

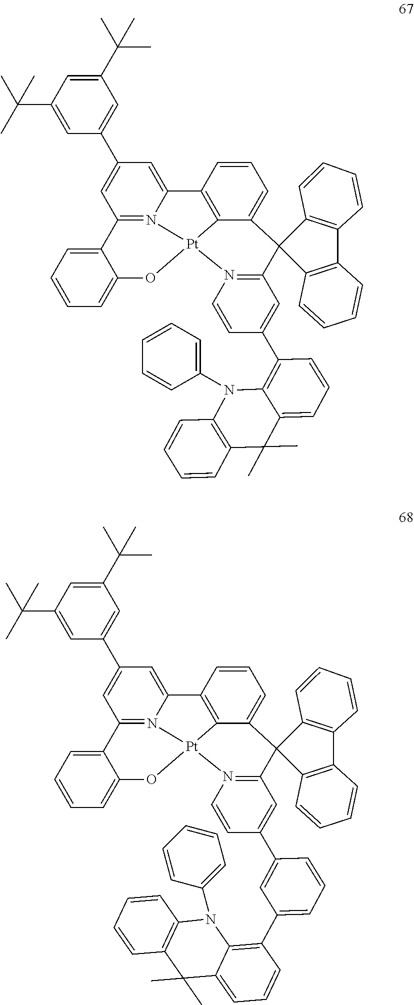

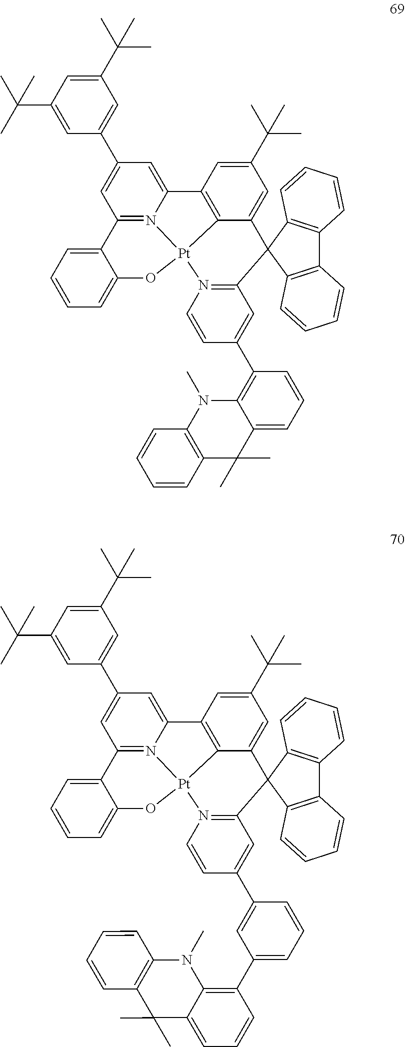

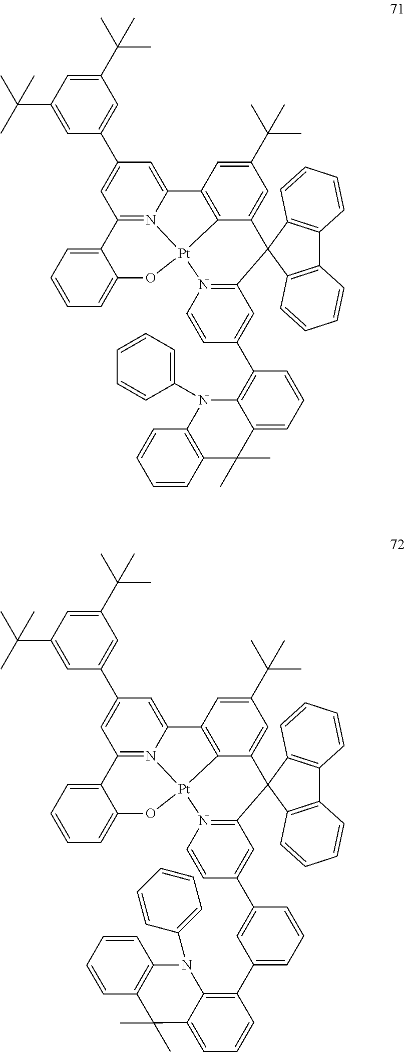

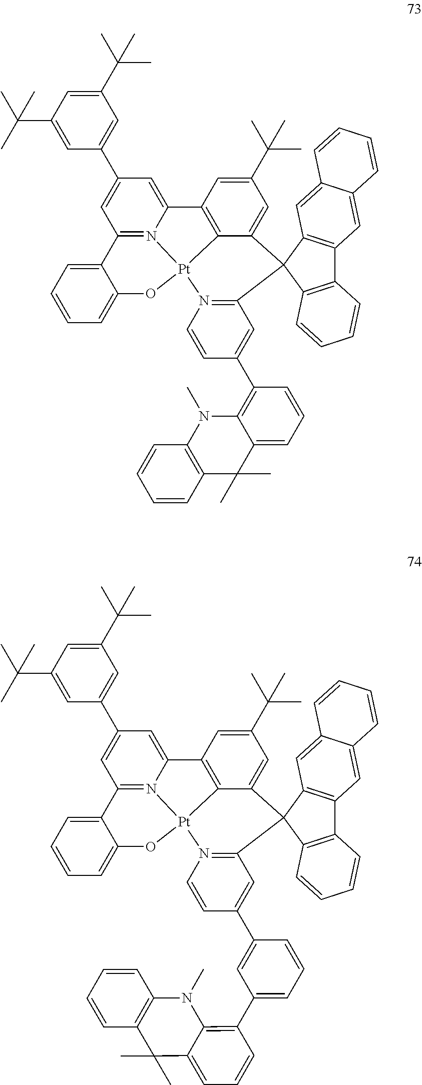

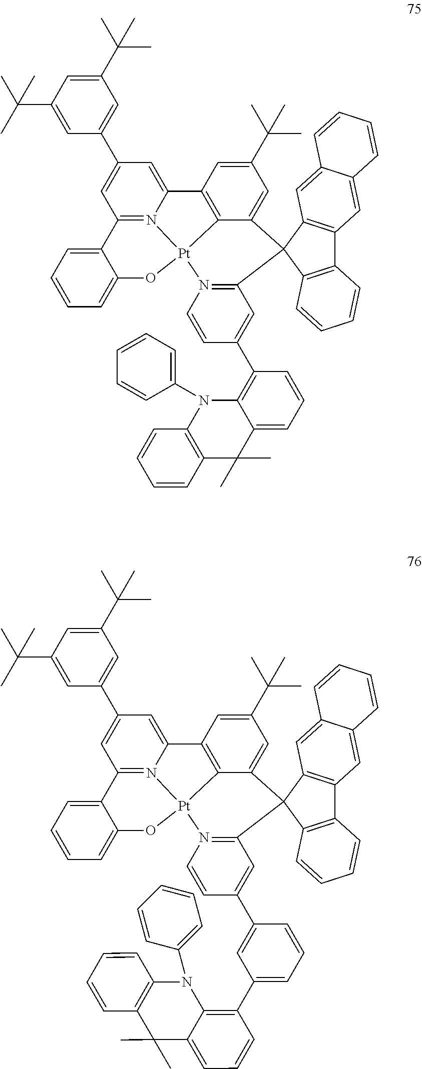

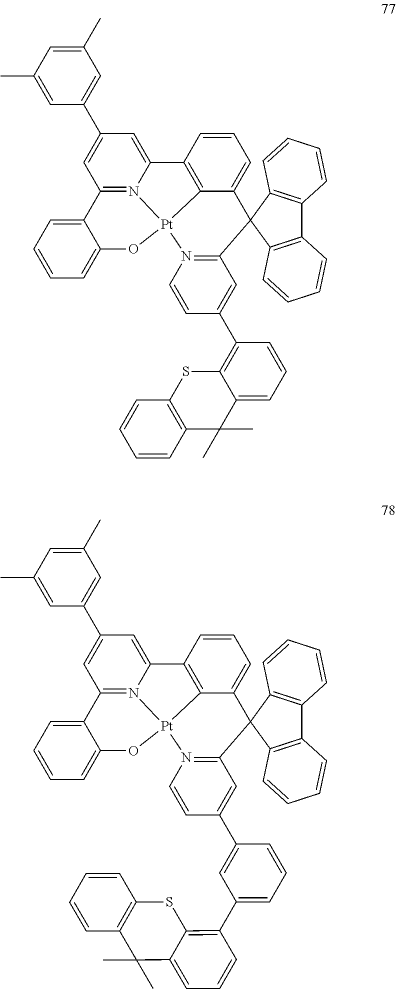

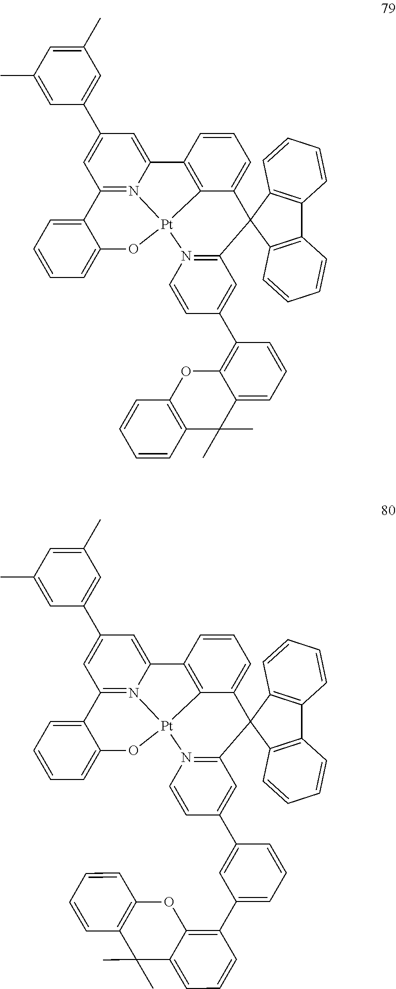

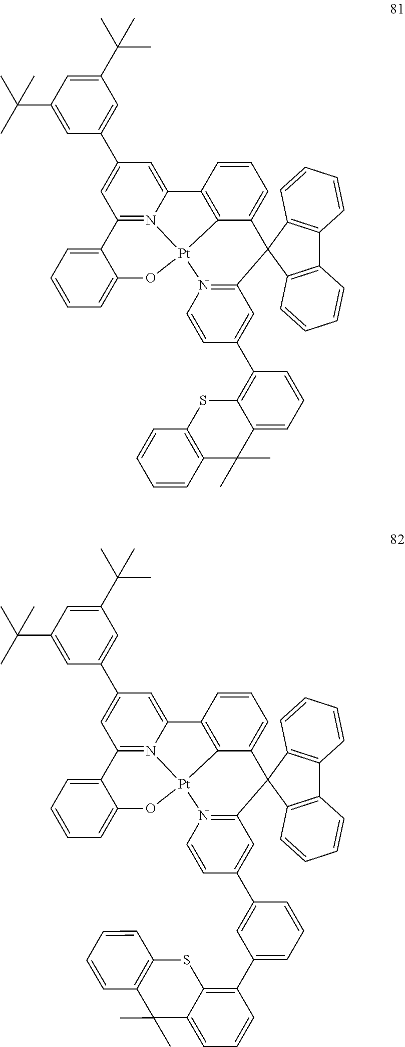

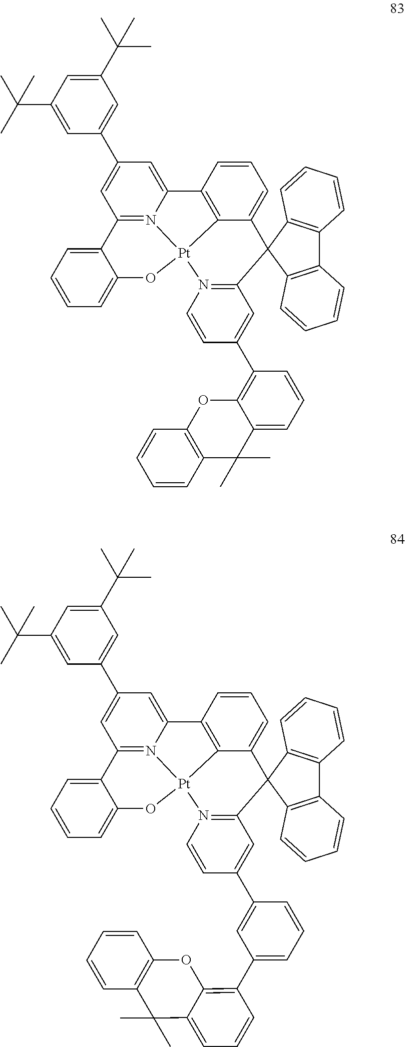

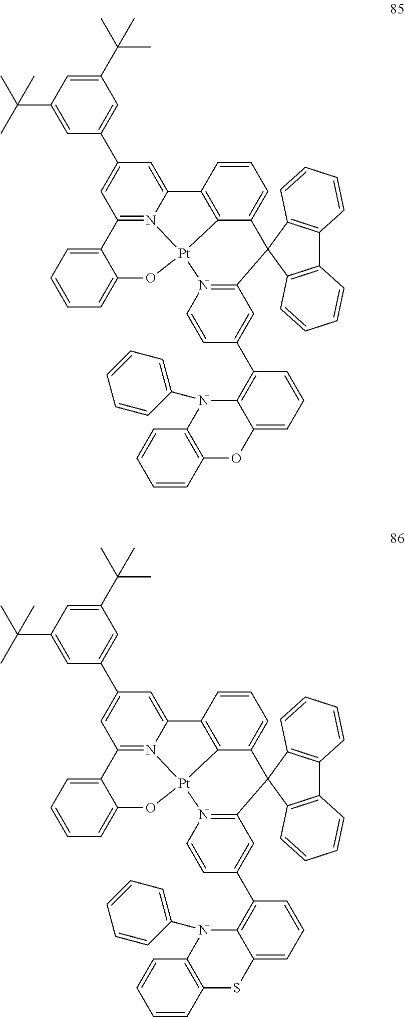

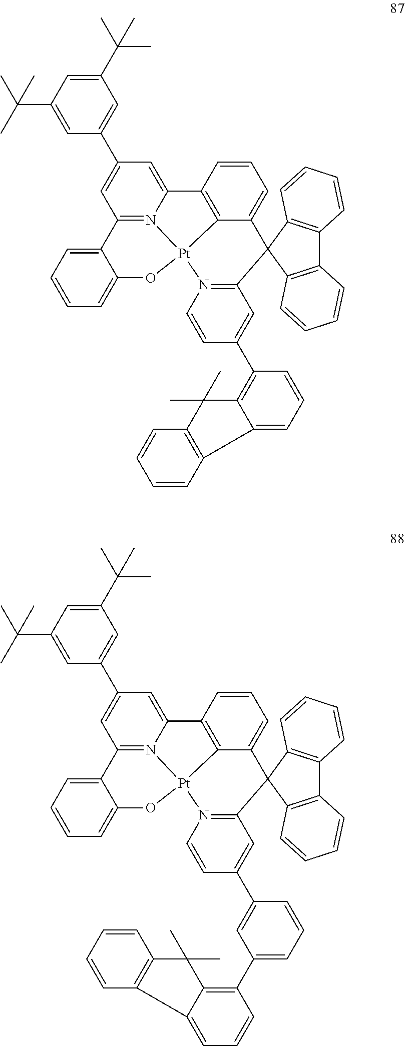

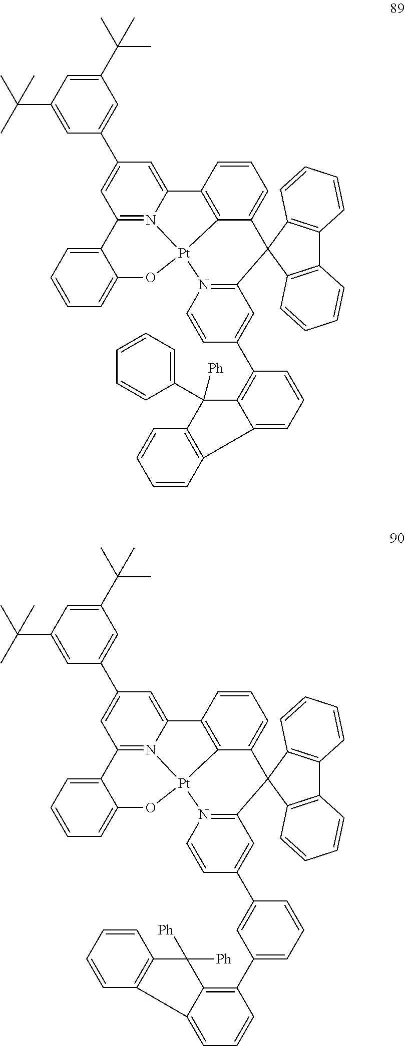

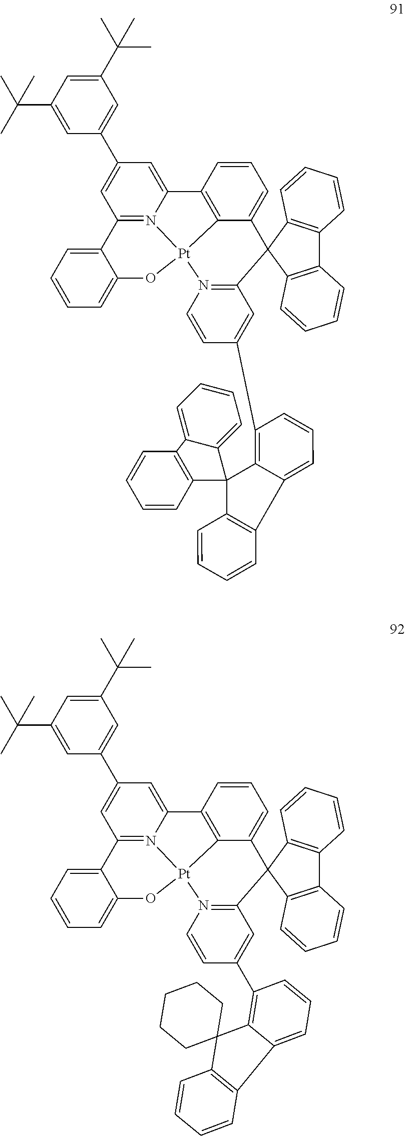

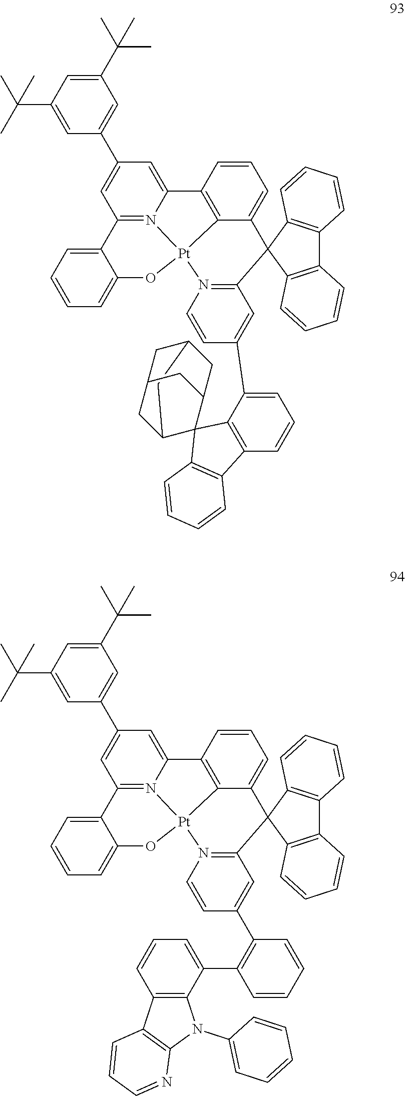

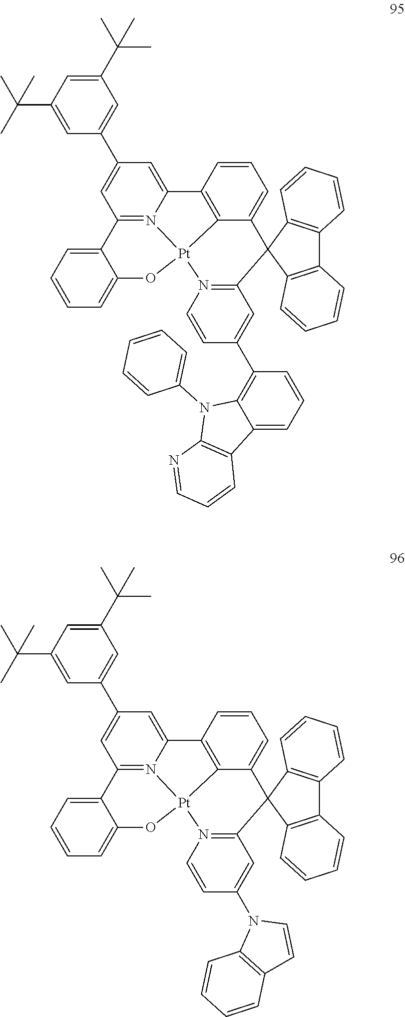

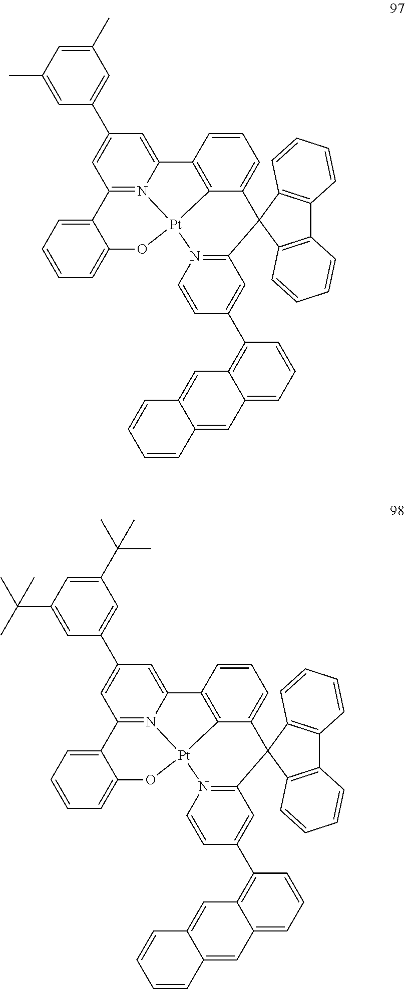

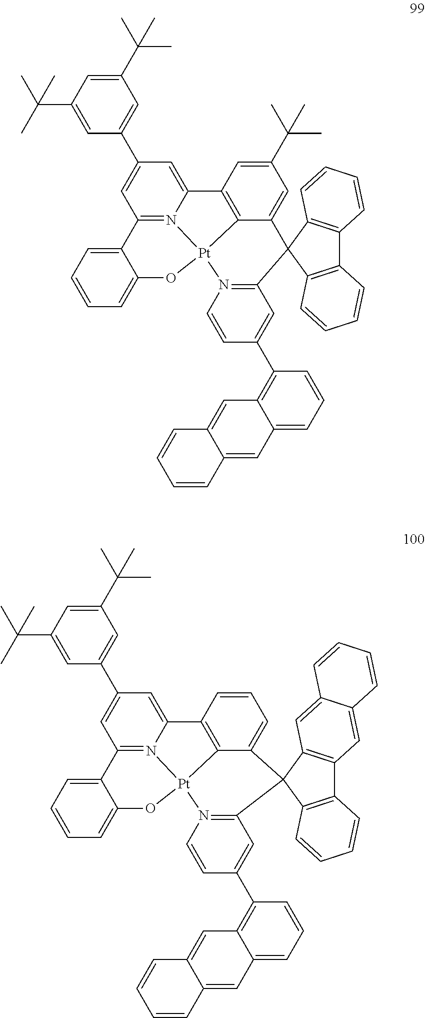

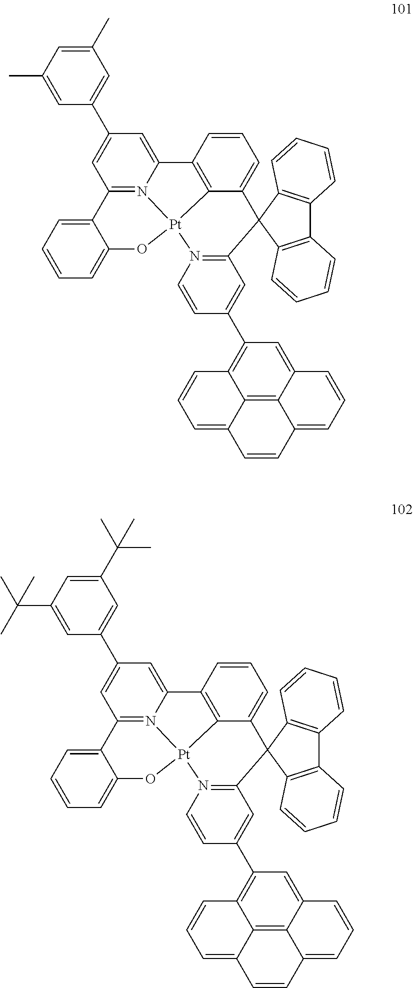

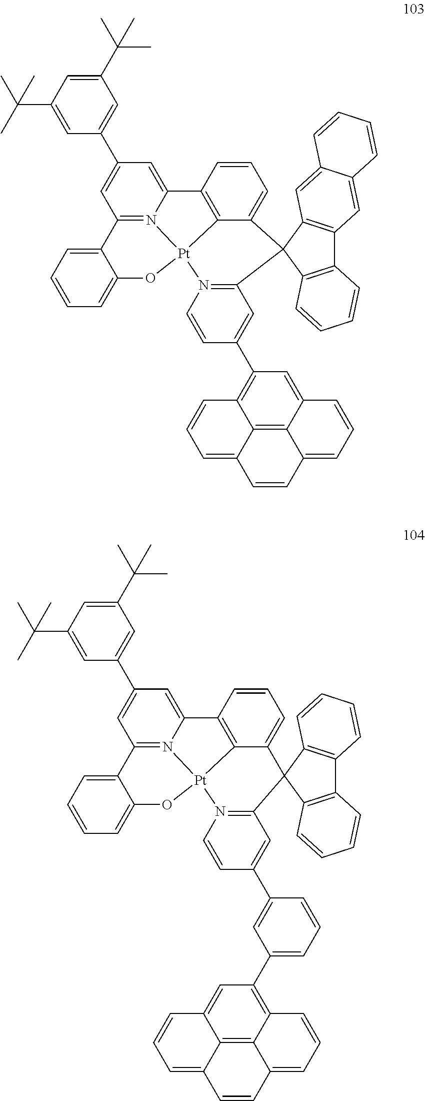

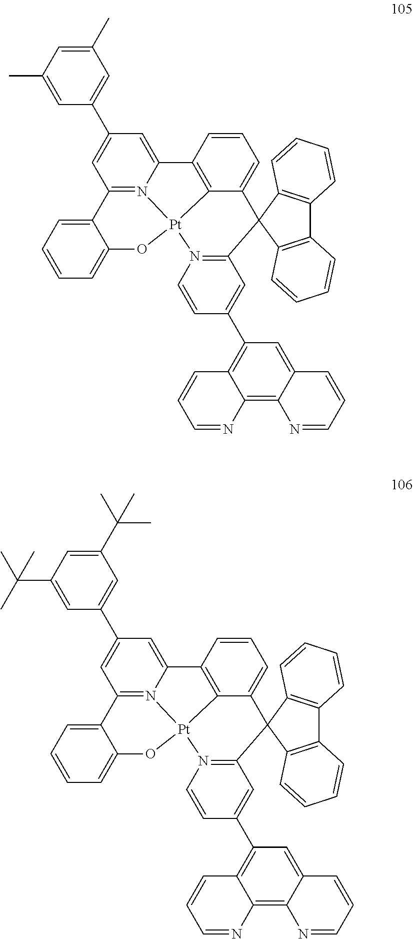

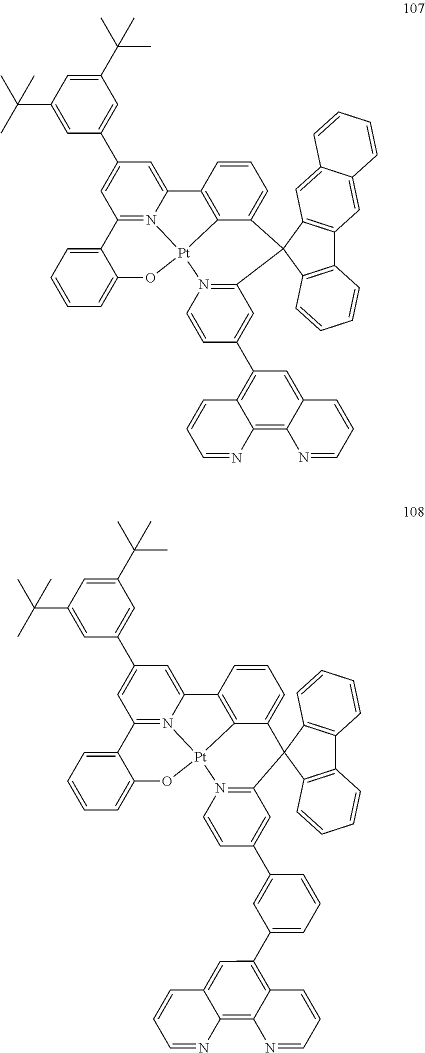

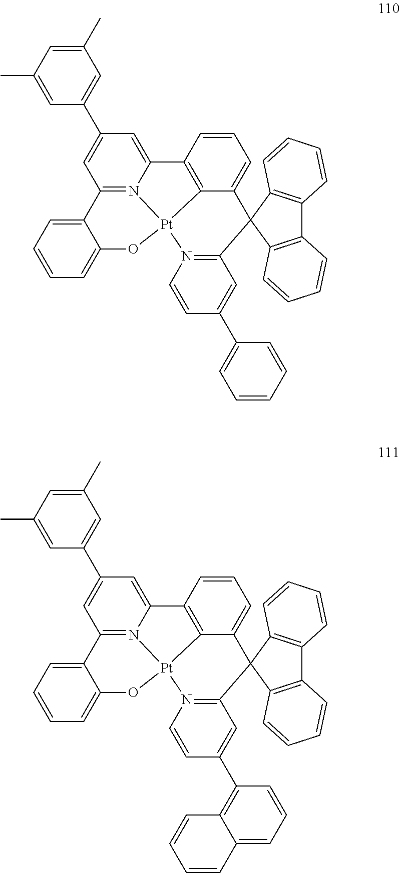

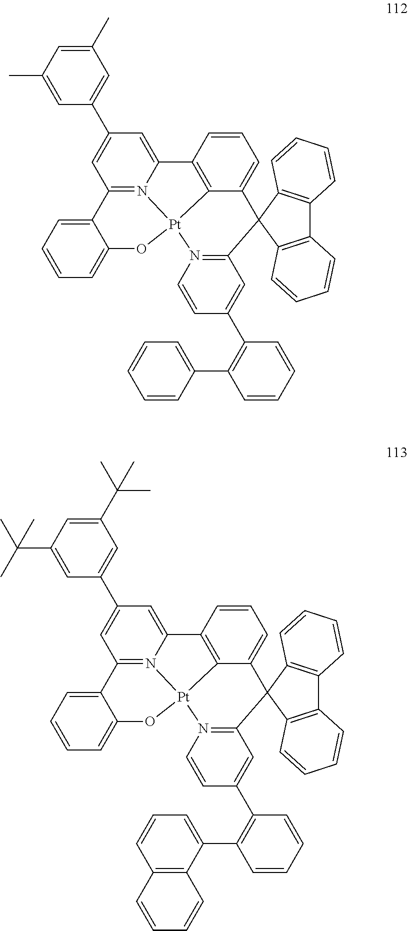

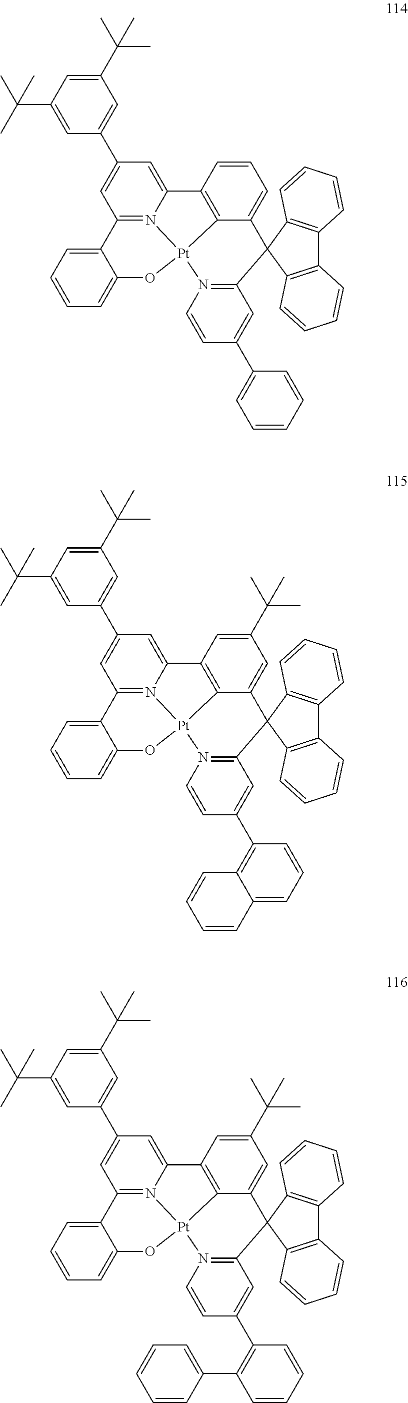

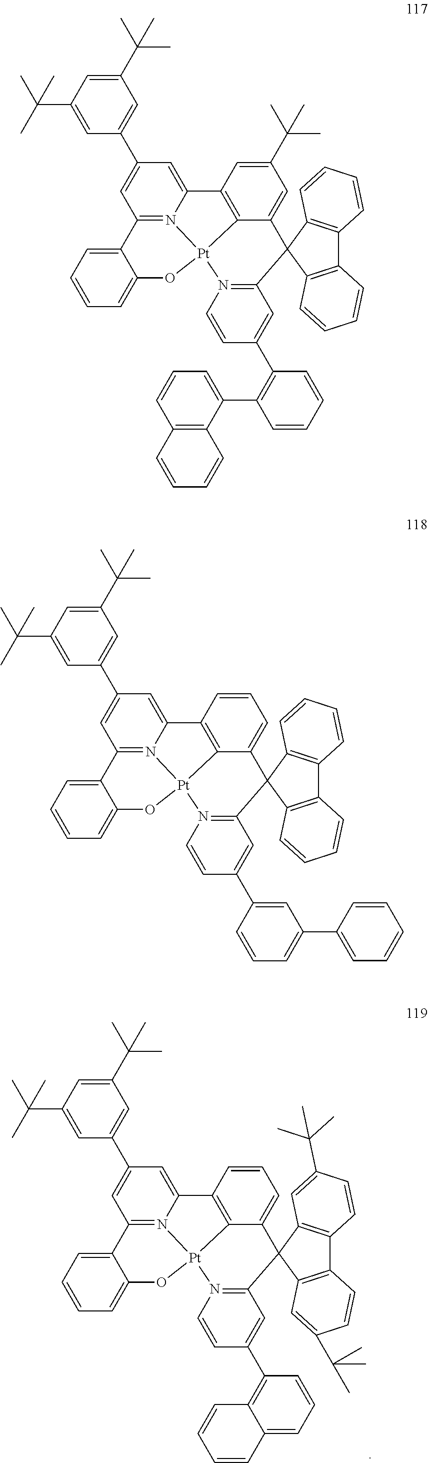

11. A precursor of the spiro-containing platinum compound according to claim 1, having a structure of formula (III): 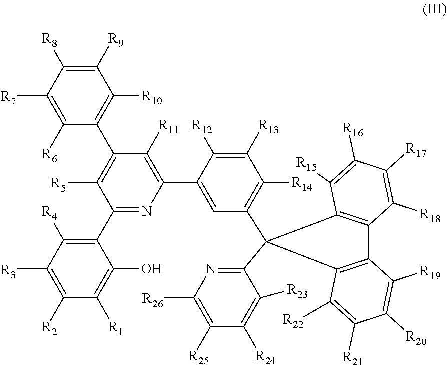
wherein R₁-R₂₆ are as defined in claim 1.

12. A precursor of the spiro-containing platinum compound according to claim 4, having a structure of formula (IV): 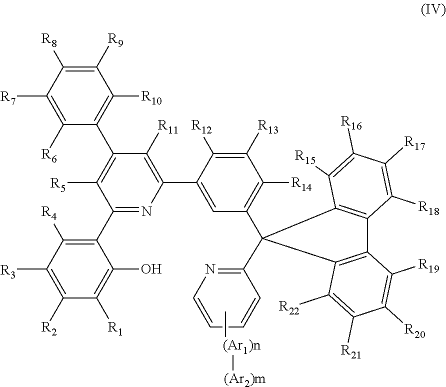
wherein Ar₁, Ar₂, n, m and R₁-R₂₂ are as defined in claim 4.

13. An organic light-emitting device, comprising a light-emitting layer, wherein the light-emitting layer comprises the spiro-containing platinum compound according to claim 1.

14. The spiro-containing platinum compound according to claim 1, wherein R₁-R₂₂ are each selected from a group consisting of hydrogen, deuterium, halogen, amino, cyano, a substituted or unsubstituted alkyl having 1 to 10 carbon atoms, a substituted or unsubstituted cycloalkyl having 3 to 10 ring carbon atoms, a substituted or unsubstituted aryl having 6 to 10 carbon atoms, a substituted or unsubstituted heteroaryl having 3 to 10 carbon atoms, a substituted or unsubstituted alkylsilyl having 3 to 10 carbon atoms, or a substituted or unsubstituted arylsilyl having 6 to 10 carbon atoms.

15. The spiro-containing platinum compound according to claim 14, wherein among R₁-R₂₂, R₇, R₉, R₁₃ and R₁₅-R₂₂ are each independently selected from a group consisting of hydrogen, deuterium, halogen, amino, cyano, or an alkyl group having 1 to 6 carbon atoms; and R₁-R₆, R₈, R₁₀-R₁₂ and R₁₄ are hydrogen.

16. The spiro-containing platinum compound according to claim 1, wherein among R₁-R₂₂, R₇, R₉, R₁₃, R₁₆, and R₂₁ are each independently selected from hydrogen, deuterium, or an alkyl group having 1 to 6 carbon atoms; and R₁-R₆, R₈, R₁₀-R₁₂, R₁₄-R₁₅, R₁₇-R₂₀ and R₂₂ are hydrogen.

17. An organic light-emitting device, comprising a light-emitting layer, wherein the light-emitting layer comprises the spiro-containing platinum compound according to claim 4.
